(19) United States
(12) Patent Application Publication
(10) Pub. No.: US 2021/0395677 A1
(43) Pub. Date: Dec. 23, 2021

(54) Microorganism-Derived Protein Hydrolysates and Methods of Preparation and Use Thereof

(71) Applicant: Air Protein, Inc., Pleasanton, CA (US)

(72) Inventors: John Reed, Pleasanton, CA (US); Dan Robertson, Pleasanton, CA (US)

(21) Appl. No.: 17/362,998

(22) Filed: Jun. 30, 2021

Related U.S. Application Data
(63) Continuation-in-part of application No. PCT/US21/14795, filed on Jan. 22, 2021.
(60) Provisional application No. 62/965,303, filed on Jan. 24, 2020.

(51) Int. Cl. C12N 5/07; C12N 1/20; C12N 5/00

(52) U.S. Cl. CPC C12N 5/06; C12N 1/20; C12N 2500/72; C12N 2501/998; C12N 2500/99; C12N 5/0031

(57) ABSTRACT

A protein hydrolysate composition derived from a microorganism, such as a chemoautotrophic microorganism, and methods of preparing and using the same are provided. The protein hydrolysate composition may be produced sustainably through fixation of carbon dioxide from biogenic or atmospheric sources. The protein hydrolysate composition finds use in supplementing culture media for serum-free culturing of animal cells as well as for growing other types of cells such as probiotics and lactic acid bacteria. Thus, the present disclosure provides sustainable, humane processes for culturing cells for pharmaceutical and nutraceutical application as well as for human consumption as a food ingredient or product, including cultured meat.

### CROSS-REFERENCE TO RELATED APPLICATIONS

This application is a continuation-in-part of PCT Application No. PCT/US21/14795, filed on Jan. 22, 2021, which claims the benefit of U.S. Provisional Application No. 62/965,303, filed on Jan. 24, 2020, both of which are incorporated by reference herein in their entireties.

### FIELD OF INVENTION

The present disclosure relates to the field of protein hydrolysates produced from biological sources, and methods of making the same. In particular, the present disclosure relates to protein hydrolysate production from renewable sources, such as biological processes designed to capture carbon dioxide emissions and other waste carbon conversion or diversion processes. The present disclosure also relates to the use of protein hydrolysates, such as hydrolysates that are derived from chemoautotrophic microorganisms, to support the growth of other microorganisms or single cells, including probiotic microorganisms, eukaryotic cells, and vitamin producing cells. The present disclosure also relates to cell culture methods for the production of meat-like products, including culturing animal cells, e.g., production of artificial meat using animal component-free media.

### BACKGROUND

Various cell culture systems are used to produce useful biological products, including small biomolecules, therapeutic antibodies, and cell-based therapies. Industrial fermentations are often performed in semi-defined and complex media that can yield higher levels of biomass or fermentation derived products. Crude and unidentified additives such as peptones, protein hydrolysates, yeast extracts, growth factors, etc., can be added to the fermentation media, to provide a broad spectrum of nutrients. The inclusion of animal-derived materials in microbiological media has a long history. Traditionally, media for culturing animal cells has incorporated animal serum or animal-derived protein hydrolysates to sustain and promote cell growth. For example, animal serum provides nutrients, hormones, growth and attachment factors, trace elements, such as iron, buffering capacity, protection against active oxygen species and mechanical shear, and various other beneficial factors. Animal protein hydrolysate serves as a nitrogen source and provides peptides and/or amino acids and vitamins.

The beneficial effect of protein hydrolysates on the growth of cell cultures, such as animal cell cultures or lactic acid bacteria (LAB) cultures, is well known, and they have served as useful cell culture additives for many decades. LAB have a limited capacity to synthesize amino acids and depend on exogenous sources of amino acids and peptides. Consequently, the provision of biochemical nitrogen sources (e.g., proteins, peptides and amino acids) is essential for the growth of LAB cultures. Typical sources of amino acids are peptides found in milk and in other hydrolyzed proteins. Protein hydrolysates from various origins are known to have different effects on various strains of LAB, with some supporting growth of an organism, and others inducing a specific type of amino acid metabolism. Different commercial protein hydrolysate products that reportedly have been used successfully for fermentations of LAB include N-Z amine, Hy-Case, Hy-Soy, Edamin, and N-Z-Case (Misono, H., Goto, N., & Nagasaki, S. (1985). Purification, crystallization and properties of NADP+-specific glutamate dehydrogenase from lactobacillus fermentum. Agricultural and Biological Chemistry. https://doi.org/10.1080/00021369.1985.10866676; Molskness, T. A., Lee, D. R., Sandine, W. E., & Elliker, P. R. (1973). -D-phosphogalactoside galactohydrolase of lactic streptococci. Applied Microbiology. https://doi.org/10.1128/aem.25.3.373-380.1973; Viniegra-Gonzalez, G., & Gomez, J. (1984). Lactic acid production by pure and mixed bacterial cultures. Bioconversion Systems. Boca Raton, Fla.; CRC Press. P, 17-39.; Vedamuthu, E. R. (1980). Method for diacetyl flavor and aroma development in creamed cottage cheese. U.S. Pat. No. 4,191,782.; Kegel, M. A., & Wallace, D. L. (1989). Use of stabilizing agents in culture media for growing acid producing bacteria. U.S. Pat. No. 4,806,479).

Tryptone (digestion of casein by the protease trypsin or pancreatic digest of casein) is a common ingredient in laboratory and fermentation media for growing wild-type and genetically modified microorganisms. The advent of recombinant DNA technology and its incorporation into the manufacture and production of bio-pharmaceutical products has resulted in an increased demand for media containing animal derived components.

Protein hydrolysates providing oligopeptides and free amino acids are widely used in animal cell culture for the production of, for example, therapeutic proteins. They have been reported to increase cell density and generate higher yields of protein products. Many commercially manufactured products, such as human growth hormone, antibiotics, insulin, etc., are produced by recombinant strains grown on nutrients and media components derived from bovine sources.

There are many different types of commercially available hydrolyzed proteins produced from a variety of sources ranging from vegetables to meat and milk. Examples of sources include caseins, whey proteins, skim milk, milk and whey protein isolates and concentrates, lactalbumin, meat and animal tissue, collagen, gelatin, corn, cottonseed, soy meal, soy protein isolates and concentrates. Some of these materials are by-products of other processes and are highly variable. In other cases, even though a particular source of protein (e.g., casein and whey) can have a fairly constant composition of amino acids, various protein hydrolysates from the same source still can have significant variations in peptide and amino acid profiles. Also, different protein sources such as casein, meat, gelatin, and soy, etc. differ from each other in their total amino acid composition and protein structure. Dairy hydrolysates are abundant in amino acids such as Glu, Ile, Leu, Val, and Met, whereas meat/soy hydrolysates are relatively more abundant in Arg, Cys, Gly, and Pro.

Animal-derived hydrolysates that have historically found use as cell culture nutrient additives for production of pharmaceuticals such as vaccines and biologicals including lactalbumin hydrolysate, casein hydrolysate, and primatone. However, such animal-derived growth supplements have downsides. The composition of serum and protein hydrolysates is not well-defined, and the supply of serum is prone to batch-to-batch variation. Because of its animal origin, serum comes with a risk of contamination by adventitious agents and contaminants, such as prions. This has been a major motivation for eliminating serum from the cell culture processes. Concerns in the biopharmaceutical industry and regulatory agencies over prions, such as transmissible spongiform encephalopathies (TSEs), and other adventitious agents, such as viruses and mycoplasma, from the use of animal-derived components have prompted the elimination of animal-derived components from media. With the emergence of Bovine Spongiform Encephalopathy (BSE) and the consequent increase in Food and Drug Administration (FDA) regulations, efforts to eliminate materials of bovine origin from fermentation media are underway. Serum is also expensive to produce. Other factors, such as serum availability, degradation of target molecules by serum proteases, impact on complexity of downstream product purification, potential immunogenicity, and other serum-associated artifacts, are also concerns. Further, there is increasing interest in the use of humane and sustainable methods for manufacturing biological products. For example, there is increasing interest in animal-free, sustainable methods of culturing meat products.

Traditionally, mammalian cells have generally required a nutrient mixture (basal medium) supplemented with 5-20% of serum or serum-derived components. Fetal calf serum (FCS), also called fetal bovine serum (FBS), is a rich source of medium component substances including hormones (e.g., insulin), growth factors, transport proteins (such as transferrin and albumin), nutrients, and attachment factors. Serum has traditionally been included in growth media as a source of nutrients, hormones, growth factors, and protease inhibitors. It facilitates the attachment and spreading of animal cells and provides non-specific protection against mechanical damage and shear forces, binds toxic compounds and improves the buffering capacity of the medium. Albumin and transferrin in serum act as carriers of lipids, fatty acids, hormones, and trace elements such as iron.

To avoid ingredients of animal origin, suppliers of media, feed, and ingredients have responded by developing media that use ingredients of plant origin, or which are manufactured through recombinant expression. Protein hydrolysates (e.g., peptones) have been used to replace serum and serum components in a variety of mammalian culture processes for decades. Plant-derived protein hydrolysates from, for example, cottonseed, soy, or wheat, used individually or in combination, have been found to be suitable alternatives to bovine serum in certain cell culture systems. Relatively crude lysates and extracts of organs, tissues and proteins from animal origin that have been grandfathered in historical manufacturing processes, are being replaced by more reproducible yeast and plant-derived substitutes. Milk and meat hydrolysates in many cases have also been replaced by plant and yeast-derived hydrolysates.

Plant derived protein hydrolysates can contain a mixture of oligopeptides, free amino acids, carbohydrates, vitamins, minerals such as potassium, calcium, iron, and trace minerals, lipids, nucleosides, nucleotides, trace low molecular weight components and undefined components. Protein hydrolysates can increase both cell growth rate, recombinant protein secretion, and long-term cell viability. Protein hydrolysates can supply nutrients, adhesion components, and/or growth factor analogues. Protein hydrolysates are used as key medium additives in serum-free cell culture processes for industrial production of therapeutic recombinant proteins. The beneficial effect of plant-derived hydrolysates on industrially-important cell types such as hybridoma, African green monkey kidney (VERO), Chinese Hamster Ovary (CHO), Human Embryonic Kidney (HEK), Baby Hamster Kidney (BHK), and baculovirus-infected Spodoptera frugiperda insect cells (Sf9), is well documented. Protein hydrolysates are reported to stimulate a more efficient cell metabolism with a more efficient use of amino acids, often improving cellular metabolism beyond that attained with serum.

However, there still is a continued need for new types of animal-free cell culture medium supplements. Some plant derived hydrolysates have demonstrated poor performance in certain applications. Previous studies have demonstrated that wheat gluten extracts or extracts with high free amino acid content may be toxic or induce toxic effects in certain cell types in vitro and can inhibit protein synthesis in cell-free systems of animal cells. There are concerns about pesticide and herbicide residues in some plant-sourced hydrolysates. To be used in commercially successful fermentations, media ingredients need to be inexpensive, readily available, and of reproducible quality. Unfortunately, many complex nutrients, including animal and plant derived protein hydrolysates, extracts, and serums are subject to large inconsistencies due to variations in of biological sources, and variation of these materials by region, climate, and in different seasons. These variations can significantly impact the quality of the final fermentation product, resulting in significant variation in outcomes and production losses. There is a demand for complex nutrients, such as lysates, protein hydrolysates, and peptide compositions, that are more consistent, and which aren't subject to regional, climatic, or seasonal variations. There is a need for protein hydrolysate products having more consistent peptide and amino acid profiles. There is a demand for non-animal sourced protein hydrolysates as a replacement of animal derived media components including animal derived media nutrient components, protein hydrolysates, and serum. There is a need to minimize variability between batches of culture media and/or culture media supplements. There is also a demand for protein hydrolysates that are free of any pesticide, herbicide, fungicide, hormone, or antibiotic residues. Since animal derived components may contain infectious agents, there is an interest in developing media that have minimal animal derived components, or which are completely animal-free. There also is an ethical and environmental demand to minimize the use of animal derived products.

Mammalian cell culture is used industrially to produce antibody and small molecule therapeutics. Traditional cell culture media have incorporated animal serum or animal-derived hydrolysates as a compositionally undefined growth sustaining component. The animal serum is a source of nutrients, hormones, and trace elements, especially iron. However, there are downsides to using serum, such as the potential for contaminating adventitious agents (prions, etc.), cost of production, and variable supply and composition.

The pharmaceutical industry has replaced serum with protein hydrolysates from plant and microbial biomass, e.g., soy, yeast. These alternatives to serum include proteins, peptides, amino acids, lipids, and carbohydrates, which are available to support and stimulate cultured cell growth. Studies have demonstrated the value and limitations of these products in cell culture media. (Pasupuleti, V. K., et al. (2010) “Applications of protein hydrolysates in biotechnology,” in Protein Hydrolysates in Biotechnology, Springer, 1-9; Lobo-Alfonso, J., et al. (2010) “Benefits and limitations of protein hydrolysates as components of serum-free media for animal cell culture applications,” in Protein Hydrolysates in Biotechnology, Springer, 55-78; Spearman, M., et al. (2014) Biotechnol Prog 30:584-593; Sung, et al., (2004) Appl Microbiol Biotechnol 6:527-536)

The burgeoning demand for animal-free, sustainable meat production is driving the adoption of production methods for consumable meats derived from cultured cells of chicken, beef, pork, and fish. (Memphis Meats, https://www.memphismeats.com; Higher Steaks, https://www.highersteaks.com; Impossible Foods, https://www.impossiblefoods.com; Finless Foods, https://www.finlessfoods.com; New Harvest, https://new-harvest.org) These markets will require scaled economic production using animal-free media.

Enzymatic hydrolysis can result in unique peptide compositions. Hydrolysates with peptide size distribution (DP 2-10) have been shown to be energetically favored carbon and nitrogen sources in animal cell culture. (Franek, F. (2010) “Oligopeptides as external molecule signals affecting growth and death in animal cell cultures,” in Protein Hydrolysates in Biotechnology, Springer, 79-89) Also, some peptide sequences act as external molecule signals.

Use of compositions derived from plants or yeast in media for production of therapeutic proteins in cultured animal cells have been described. (U.S. Pat. Nos. 7,955,833, 8,093,045, and 8,440,408, and PCT Application Nos. WO1999/057246 and WO2004/078955) Hydrolysates derived from plant or yeast sources are produced in processes that rely on land/water-intensive agriculture or sugar-based fermentations. Improved methods for production and use of non-animal based protein hydrolysates in culture media would be desirable.

### SUMMARY OF THE INVENTION

Provided herein are protein hydrolysate compositions derived from microorganisms grown by fermentation on an organic carbon source or grown chemoautotrophically. In certain embodiments, microorganisms are grown on a C1 carbon source, such as: chemoautotrophic growth on CO₂ as a carbon source; carboxydotrophic growth on CO as a carbon source; or methanotrophic or methylotrophic growth on CH₄ and/or CH₃OH as a carbon source.

The protein hydrolysates described herein are suitable as a replacement for all or part of an animal component supplement for promoting the proliferation, maintenance, propagation and/or differentiation of animal cells in culture in the absence of one or more animal-derived cell culture components, such as amino acids, protein hydrolysates, or serum. For example, the protein hydrolysates may be suitable for use as an animal component free supplement, e.g., in an animal or serum-free medium. Lysates and/or hydrolysates produced as described herein can provide growth promoting peptides, amino acids, carbohydrates, lipids, minerals, and/or vitamins. In certain embodiments, the peptides produced as described herein serve one or more of the following functions: as an amino acid source (e.g., replacing or supplementing free amino acids); as a stimulator of growth and/or production of biomass and/or molecules of interest; in the protection of cells against shear stress. In certain embodiments, the lysates and/or hydrolysates described herein stimulate a more efficient cell metabolism through mechanisms including, but not limited to, more efficient uptake and use of amino acids. In certain embodiments, the lysates and/or hydrolysates improve the growth rate and/or protein expression in a culture that is grown on a medium that includes the lysate and/or hydrolysate, in comparison to a culture that does not include the lysate and/or hydrolysate. In certain embodiments, the lysates and/or hydrolysates improve the performance of cultures grown on a medium that includes the lysate and/or hydrolysate, through effects including, but not limited to, osmo-protectant and/or anti-apoptotic effects. In certain embodiments, the lysates and/or hydrolysates help protect against shear effects. In certain embodiments. the lysates and/or hydrolysates promote attachment and/or subsequent spreading of cells.

The present disclosure includes animal component-free protein hydrolysates suitable for use in the production of consumable meat products derived from cultured animal cells. Also, the present disclosure includes animal component-free protein hydrolysates suitable for the growth of organisms that are “generally recognized as safe” (GRAS), and/or probiotic microorganisms, which can also be processed into meat-like and/or high protein products, as well as other food products, ingredients, nutrients, and flavors. Also, the present disclosure includes animal component-free protein hydrolysates suitable for the growth of organisms that are considered beneficial members of a plant or animal microbiome. In certain embodiments, the plant or animal is used in the production of human food or animal feed or other plant- or animal-derived products that are used by humans. The animal component-free protein hydrolysates may be generated sustainably, e.g., from microbial fixation of CO₂ from sustainable and clean sources, such as CO₂ off gas from fermentations at a brewery or winery, as well as CO₂ captured from the atmosphere or some other natural source.

Protein hydrolysate compositions of the present disclosure may be defined by the abundance of amino acids, the size distribution of peptides, the abundance of different peptide sequences, protein identity and abundance, lipid composition, abundance of organic polymers, vitamins, minerals, etc. Provided herein is a library of protein hydrolysates where different compositions within the library are characterized by one or more of the abundance of different amino acids, the size distribution of peptides, the abundance of peptide sequences, protein identity and abundance, lipid composition, abundance of organic polymers, vitamins, minerals, etc. The protein hydrolysate composition may be suitable for promoting specific aspects (e.g., proliferation, propagation, development, differentiation) of an animal or microbial cell culture, depending on the hydrolysate composition.

The present disclosure includes protein hydrolysate compositions containing biostimulant components. In certain such embodiments, the biostimulant improves the performance or characteristics of a plant or fungal crop. In some embodiments, the protein hydrolysate composition includes a biostimulant polyester, such as a polyhydroxyalkanoate (PHA) polymer. In some embodiments, the PHA polymer may be produced by the microorganism, e.g., chemoautotrophic microorganism, from which the protein hydrolysate composition is derived. In some embodiments, the protein hydrolysate composition includes polyhydroxybutyrate (PHB). In some embodiments the protein hydrolysate composition includes polyhydroxyvalerate (PHV). In some embodiments, the protein hydrolysate composition includes co-polymers that include PHB and/or PHV In other embodiments the protein hydrolysate composition includes oligomers of PHB or PHA or PHV or co-polymers thereof. In certain embodiments, the oligomers are caused by hydrolysis of the respective PHB or PHA or PHV containing polymer. In certain embodiments the protein hydrolysate composition includes hydroxybutrate (HB) or hydroxyvalerate (HV) monomers.

The present disclosure includes protein hydrolysate compositions containing one or more vitamins. The vitamin may be produced by the chemoautotrophic microorganism from which the protein hydrolysate composition is derived. In some embodiments, the protein hydrolysate composition includes one or move B vitamins, such as vitamin B₁, vitamin B₂, and/or vitamin B₁₂. Also provided herein are methods for producing a protein hydrolysate composition from a microorganism culture, such as a chemoautotrophic microorganism culture. The composition of a protein hydrolysate of the present disclosure may vary depending on various manufacturing parameters, such as the type and strain of microorganism, e.g., chemoautotrophic microorganism, used in the culture, the growth conditions, the source of carbon and/or energy used to culture the microorganism, e.g., chemoautotrophic microorganism, the method of preparation of the protein hydrolysate, genetic engineering of the microorganism, e.g., chemoautotrophic microorganism, etc. In some embodiments, the microorganism is a chemoautotrophic microorganism, for example, any suitable chemoautotrophic microorganism that can fix carbon from a C1 carbon source, such as CO₂. In certain embodiments, CO₂ fixation is coupled to H₂ oxidation. In some embodiments, the microorganism, e.g., chemoautotrophic microorganism is genetically engineered. Or in other embodiments, the microorganism, e.g., chemoautotrophic microorganism, is not genetically engineered, and is either a naturally occurring strain, or else is a mutant or variant strain generated through one or more known methods outside of genetic engineering.

The protein hydrolysate composition may be prepared by physical, chemical and/or enzymatic treatments of a biomass generated through the growth of a microorganism, e.g., chemoautotrophic microorganism. Physical treatments include subjecting the biomass to elevated pressure and/or elevated temperature. Chemical treatments include subjecting the biomass to acidic or basic conditions. Enzymatic treatments include proteolysis with exoproteases and/or sequence-specific endoproteases and/or metalloproteases. In certain embodiments, the protein hydrolysate composition retains biostimulant components and/or vitamins after subjecting the biomass to the preparation process.

Also provided are methods of culturing cells, including animal cells, using a protein hydrolysate composition of the present disclosure as a culture media supplement. The protein hydrolysate composition may provide to the culture medium nutrients suitable to sustain growth and/or promote development and differentiation of the animal cells in culture. The method may include providing the protein hydrolysate in a culture medium and culturing animal cells in the culture medium. The protein hydrolysate composition may substitute for one or more components of a conventional cell culture medium that are typically derived from an animal source, such as amino acids, protein hydrolysate, or serum. In some embodiments, the animal cell culture medium supplemented with the protein hydrolysate composition is a serum-free culture medium. In some embodiments, the cell culture medium supplemented with the protein hydrolysate composition of the present disclosure is a vegan culture medium. In some embodiments, the cell culture medium, including but not limited to animal cell culture medium, which is supplemented with the protein hydrolysate composition, is an animal component-free culture medium. In some embodiments, a lysate, protein hydrolysate, and/or amino acid composition produced from a microorganism, e.g., chemoautotrophic microorganism, is provided along with serum to an animal cell culture. In certain embodiments, a lysate, protein hydrolysate, and/or amino acid composition is used for the growth of attachment-dependent cells. In other embodiments, a lysate, protein hydrolysate, and/or amino acid composition is used for the growth of attachment-independent cells.

The protein hydrolysate composition may be suitable for use in the culture medium for culturing cells for a variety of purposes, including recombinant cell lines for expressing therapeutic antibodies, proteins or small molecules, or immune cells cultured for cell-based therapy. In some embodiments, the protein hydrolysate composition may be suitable for use in a culture medium for culturing a meat product, including, but not limited to, an artificial meat or meat substitute product. In some embodiments, the protein hydrolysate composition may be suitable for use in a culture medium for culturing probiotic or vitamin synthesizing microorganisms.

### BRIEF DESCRIPTION OF THE DRAWINGS

FIG. 1 is a schematic diagram of a method according to embodiments of the present disclosure for producing and extracting nutrients from a first bioprocess, that is utilized within a nutrient medium for a second cell culture.

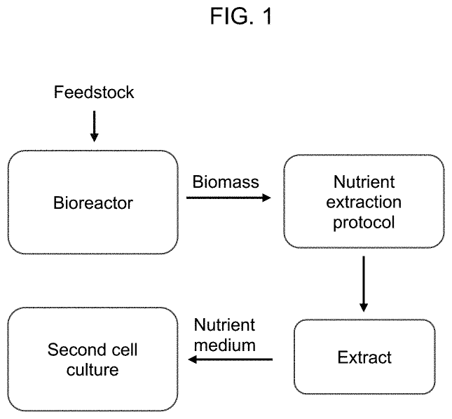

FIG. 2 is a schematic diagram of a method according to embodiments of the present disclosure for chemoautotrophically producing protein-rich biomass from CO₂, and then converting the protein-rich biomass to a protein hydrolysate which is utilized within a nutrient medium for a second cell culture.

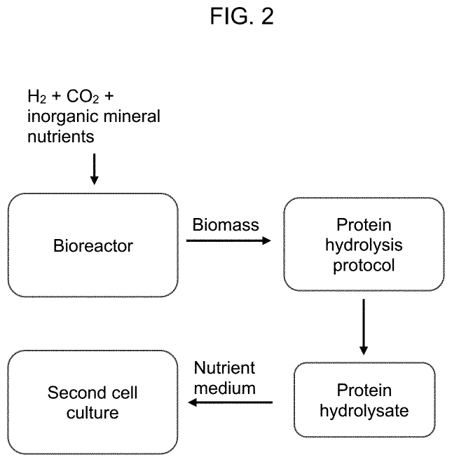

FIG. 3 is a schematic diagram of a method according to embodiments of the present disclosure for chemoautotrophically producing protein-rich biomass from CO₂, and then converting the protein-rich biomass to a protein hydrolysate which is utilized within a nutrient medium for an animal cell culture, from which animal cells are grown and recovered to produce cultured meat, i.e., meat products that don't involve killing whole multicellular animals.

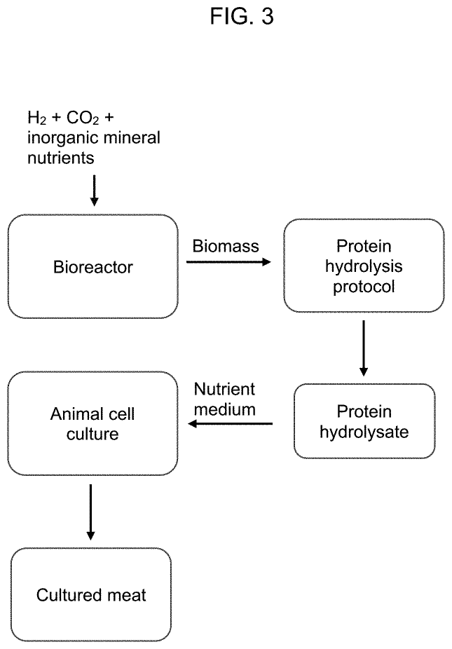

FIG. 4 is a schematic diagram of a method according to embodiments of the present disclosure for chemoautotrophically producing protein-rich biomass from CO₂, and then converting the protein-rich biomass to a protein hydrolysate which is utilized within a nutrient medium for a microbial culture that comprises probiotics and/or beneficial microorganisms to humans, plants, animals.

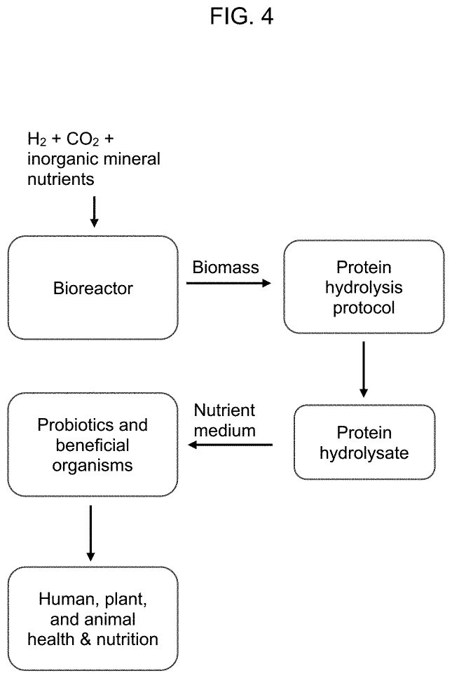

FIG. 5 shows growth of the beneficial fungus Trichoderma atroviride on medium supplemented with protein hydrolysate (PH) produced from C. necator grown on CO₂ as carbon source, compared to control growth of Trichoderma atroviride without PH supplement.

### DETAILED DESCRIPTION

A protein hydrolysate composition derived from a microorganism, e.g., chemoautotrophic microorganism, and methods of preparing and using the same are provided. The microorganism-derived protein hydrolysate compositions may be produced from microbial biomass and may be used as a media supplement to provide nutrient and support for animal cells grown in culture, or may be used as nutrition for other eukaryotic and/or prokaryotic cells, such as, for example, lactic acid bacteria (LAB) and/or probiotics. Like other plant-based or yeast-based protein hydrolysates, the present microorganism-derived protein hydrolysates may be used to supplement culture media for propagating, differentiating and/or developing cells, such as eukaryotic cells (e.g., animal cells) and/or prokaryotic cells (e.g., LAB and/or probiotic cells), without relying on animal-derived components, such as serum or growth factors.

Protein hydrolysates from chemoautotrophic microorganisms differ from those from animals or plants in that they may contain biostimulants, e.g. polyhydroxyalkanoate (PHA) biopolymers, e.g., polyhydroxybutyrate (PHB), essential vitamins, e.g., that act as enzyme co-factors, such as vitamin B12, and unique trace elements. Selective enzymatic hydrolysis can favor peptide length distribution and/or enrich for bioactive peptide sequences, creating fortified hydrolysate compositions.

Methods are provided herein for production of unique protein hydrolysate compositions from chemoautotrophic microorganisms, or fractions derived therefrom, that supply essential nutrients and/or molecular signals, e.g., bioactives, required from cultured cell growth and yield. The methods have application in the production of humane (e.g., cultured) meat products.

The methods described herein deploy biomass derived from chemoautotrophic microorganisms. In some embodiments, the microorganisms are grown on CO₂ derived from industrial flue gas or waste gasification as a carbon source.

In some embodiments, the chemoautotrophic microorganisms may include, but are not limited to, Aquifex pyrophilus, Aquifex aeolicus, or other Aquifex sp.; Cupriavidus necator or Cupriavidus metallidurans or other Cupriavidus sp.; Corynebacterium autotrophicum or other Corynebacterium sp.; Gordonia desulfuricans, Gordonia polyisoprenivorans, Gordonia rubripertincta, Gordonia hydrophobica, Gordonia westfalica, or other Gordonia sp.; Nocardia autotrophica, Nocardia opaca, or other Nocardia sp.; purple non-sulfur photosynthetic bacteria, including but not limited to, Rhodobacter sphaeroides, Rhodopseudomonas palustris, Rhodopseudomonas capsulate, Rhodopseudomonas viridis, Rhodopseudomonas sulfoviridis, Rhodopseudomonas blastica, Rhodopseudomonas spheroides, Rhodopseudomonas acidophila, or other Rhodopseudomonas sp.; Rhodobacter sp., Rhodospirillum rubrum, or other Rhodospirillum sp.; Rhodococcus opacus or other Rhodococcus sp.; Rhizobium japonicum or other Rhizobium sp.; Thiocapsa roseopersicina or other Thiocapsa sp.; Pseudomonas facilis, Pseudomonas flava, Pseudomonas putida, Pseudomonas hydrogenovora, Pseudomonas hydrogenothermophila, Pseudomonas palleronii, Pseudomonas pseudoflava, Pseudomonas saccharophila, Pseudomonas thermophile, or other Pseudomonas sp.; Hydrogenomonas pantotropha, Hydrogenomonas eutropha, Hydrogenomonas facilis, or other Hydrogenomonas sp.; Hydrogenobacter thermophiles, Hydrogenobacter halophilus, Hydrogenobacter hydrogenophilus, or other Hydrogenobacter sp.; Hydrogenophilus islandicus or other Hydrogenophilus sp.; Hydrogenovibrio marinus or other Hydrogenovibrio sp.; Hydrogenothermus marinus or other Hydrogenothermus sp.; Helicobacter pylori or other Helicobacter sp.; Xanthobacter autotrophicus, Xanthobacter flavus, or other Xanthobacter sp.; Hydrogenophaga flava, Hydrogenophaga palleronii, Hydrogenophaga pseudoflava, or other Hydrogenophaga sp.; Bradyrhizobium japonicum or other Bradyrhizobium sp.; Ralstonia eutropha or other Ralstonia sp.; Alcaligenes eutrophus, Alcaligenes facilis, Alcaligenes hydrogenophilus, Alcaligenes latus, Alcaligenes paradoxus, Alcaligenes ruhlandii, or other Alcaligenes sp.; Amycolata sp.; Aquaspirillum autotrophicum or other Aquaspirillum sp.; Arthrobacter strain 11/X, Arthrobacter methylotrophus, or other Arthrobacter sp.; Azospirillum lipoferum or other Azospirillum sp.; Variovorax paradoxus or other Variovorax sp.; Acidovorax facilis, or other Acidovorax sp.; Bacillus schlegelii, Bacillus tusciae, other Bacillus sp.; Calderobacterium hydrogenophilum or other Calderobacterium sp.; Derxia gummosa or other Derxia sp.; Flavobacterium autothermophilum or other Flavobacterium sp.; Microcyclus aquaticus or other Microcyclus sp.; Mycobacterium gordoniae or other Mycobacterium sp.; Paracoccus denitrificans or other Paracoccus sp.; Persephonella marina, Persephonella guaymasensis, or other Persephonella sp.; Renobacter vacuolatum or other Renobacter sp.; Seliberia carboxydohydrogena or other Seliberia sp.; Streptomycetes coelicoflavus, Streptomycetes griseus, Streptomycetes xanthochromogenes, Streptomycetes thermocarboxydus, or other Streptomycetes sp.; Thermocrinis ruber or other Thermocrinis sp.; Wautersia sp.; cyanobacteria including, but not limited to, Anabaena oscillarioides, Anabaena spiroides, Anabaena cylindrica, or other Anabaena sp.,; Arthrospira platensis, Arthrospira maxima, or other Arthrospira sp.; green algae including, but not limited to, Scenedesmus obliquus or other Scenedesmus sp., Chlamydomonas reinhardii or other Chlamydomonas sp., Ankistrodesmus sp.; Rhaphidium polymorphium or other Rhaphidium sp.; as well as a consortium of microorganisms and/or organisms that includes oxyhydrogen microorganisms. In some embodiments, the chemoautotrophic microorganisms include a Cupriavidus species, such as Cupriavidus necator, for example, but not limited to, C. necator strain DSM 531 or DSM 541.

In some embodiments, chemoautotrophic microorganisms may be cultured autotrophically, e.g., by CO₂ fixation coupled with H₂ oxidation, to generate a biomass, for example, from a clean and sustainable source of CO₂, such as off-gas from fermentations at a brewery or winery, or CO₂ captured from the atmosphere, or the ocean, or from other natural sources such as geothermal vents or hot springs. The utilization of chemoautotrophic microorganisms enables broad flexibility in choosing the source of carbon as feedstock for production; from fermentation off-gas, to natural sources of CO₂, to industrial flue gas, to gasification of biomass, e.g., agriculture or forestry wastes.

Therefore, the present disclosure provides a sustainable source of protein hydrolysates that may be used to support humane methods for culturing animal cells and for producing food products with animal proteins and other nutrients.

The present disclosure provides methods for producing a culture medium for culturing microorganisms or cells, comprising: culturing a first microorganism, and thereby producing biomass; processing the biomass generated by the first cultured microorganism to produce a protein-rich biomass derived product, such as, but not limited to a protein-rich lysate or protein hydrolysate composition; and adding the protein-rich nutrient (e.g., protein-rich lysate or protein hydrolysate) composition to a culture medium for a second culture comprising a second microorganism or cells, wherein the protein-rich nutrient (e.g., protein-rich lysate or protein hydrolysate) composition serves as a nutrient source for the growth of the second microorganism or cells. In certain non-limiting embodiments, the second culture comprises animal cells.

In some embodiments, the present protein hydrolysate composition may be used as a media supplement for culturing a food product, such as a meat product, and thereby supports sustainable, humane methods of producing food products, such as cultured meat.

In some embodiments, the protein hydrolysate composition of the present disclosure may include biostimulant components, such as polyhydroxyalkanoate (PHA) polymers, that promote cell proliferation, maintenance, development and/or differentiation, in contrast to plants, which generally do not produce PHAs. The PHA, PHA oligomers, and monomers produced in the present invention can act as nutrients and bioactives.

In some embodiments, the present protein hydrolysate composition includes one or more vitamins, such as, but not limited to vitamin B₁, B₂, and/or vitamin B₁₂, which may promote cell culture growth, development and/or differentiation, and/or improve the nutritional value of a cultured food product when the culture media is supplemented with the protein hydrolysate. Hydrolysates made as described herein can contain unique nutrients which are not synthesized by plants, and which generally are not present in appreciable quantities in plants, such as, but not limited to vitamin B₁₂.

In some embodiments, the present protein hydrolysate composition may improve or enhance the flavor of a cultured food product when provided in the culture medium. In some embodiments, the present protein hydrolysate composition has nutritional benefits that are comparable or superior to animal, plant, and yeast hydrolysates and extracts.

The present disclosure provides methods of manufacturing protein hydrolysates, lysates, and/or extracts with a number of benefits including, but not limited to: consistent and totally defined initial inputs starting with the chemoautotrophic conversion of CO₂ and/or other C1 inputs into protein-rich biomass; compositional consistency; large scale production capacity with a small footprint; no agricultural land displacement; lower water use vis-à-vis plant- or animal-derived hydrolysates. In certain embodiments of the present disclosure, no arable land is required in the production of the protein hydrolysates, lysates, and/or extracts produced as described herein.

The present disclosure provides acid, base, and enzymatic hydrolysis methods, producing hydrolysates enriched in desired fractions including, but not limited to: peptides serving as a source of utilizable amino acids or exerting specific effects on plant or cell health; amino acid cleavage sequence-specific endo-proteases or protease combinations for protein digestion; controlling peptide composition to produce hydrolysates enriched in peptides of desired length; peptides with residue distributions of about 2 to about 10 amino acid residues, which improve growth and/or yield of some cultured animal cells.

Certain peptides can act as external molecular signals affecting plants, animals and microbiomes. Protein hydrolysates can provide nutrients to a plant at root level or stimulate beneficial microbial strains in the soil rhizobiome, indirectly promoting plant growth and productivity. Certain protein hydrolysates, lysates, and/or extracts of the present disclosure can stimulate organisms in a plant or animal microbiome that increase disease resistance and/or nutrient uptake.

Protein hydrolysates, lysates, and/or extracts disclosed herein can provide nutrients and bioactives to an animal via the gut or stimulate host-beneficial strains in the gut microbiome.

Use of chemoautotrophic microorganisms for production of protein and other components of food products that are suitable for human consumption, such as food products that closely mimic the properties of meat and function as meat substitutes or artificial meat products, is described in PCT Application Nos. PCT/US20/67555, filed Dec. 30, 2020, PCT/US21/20147, filed Feb. 28, 2021, and PCT/US21/23949, filed Mar. 24, 2021, all of which are incorporated herein by reference in their entireties.

Use of knallgas microorganisms for the conversion of syngas, producer gas, or other H₂ and CO₂ and/or CO containing gas mixes in high energy density molecules is described in U.S. Pat. No. 9,085,785, entitled USE OF OXYHYDROGEN MICROORGANISMS FOR NON-PHOTOSYNTHETIC CARBON CAPTURE AND CONVERSION OF INORGANIC AND/OR C1 CARBON SOURCES INTO USEFUL ORGANIC COMPOUNDS, which is incorporated herein by reference in its entirety.

Use of chemotrophic microorganisms for the conversion of CO₂ into useful organic chemicals is described in PCT application no. WO2011/056183, entitled BIOLOGICAL AND CHEMICAL PROCESS UTILIZING CHEMOAUTOTROPHIC MICROORGANISMS FOR THE CHEMOSYTHETIC FIXATION OF CARBON DIOXIDE AND/OR OTHER INORGANIC CARBON SOURCES INTO ORGANIC COMPOUNDS, AND THE GENERATION OF ADDITIONAL USEFUL PRODUCTS, which is incorporated herein by reference in its entirety.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

Singleton, et al., Dictionary of Microbiology and Molecular Biology, second ed., John Wiley and Sons, New York (1994), and Hale & Markham, The Harper Collins Dictionary of Biology, Harper Perennial, N.Y. (1991) provide one of skill with a general dictionary of many of the terms used in this invention. Any methods and materials similar or equivalent to those described herein can be used in the practice or testing of the methods, systems, and compositions described herein.

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Numeric ranges provided herein are inclusive of the numbers defining the range.

Unless otherwise indicated, nucleic acids are written left to right in 5′ to 3′ orientation; amino acid sequences are written left to right in amino to carboxy orientation, respectively.

### Definitions

“A,” “an” and “the” include plural references unless the context clearly dictates, thus the indefinite articles “a”, “an,”, and “the” as used herein in the specification and in the claims, unless clearly indicated to the contrary, should be understood to mean “at least one.”

The term “about” as used herein when referring to a measurable value such as an amount, a temporal duration, and the like, is meant to encompass variations of ±5%, ±1%, or ±0.1% from the specified value, as such variations are appropriate to perform the disclosed methods or in connection with a disclosed composition.

“Acetogen” refers to a microorganism that generates acetate and/or other short chain organic acids up to C4 chain length as a product of anaerobic respiration.

“Acidophile” refers to a type of extremophile that thrives under highly acidic conditions (usually at pH 2.0 or below).

The term “amino acid” refers to a molecule containing both an amine group and a carboxyl group that are bound to a carbon, which is designated the alpha-carbon. Suitable amino acids include, without limitation, both the D- and L-isomers of the naturally occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs. Standard three-letter abbreviations for amino acids are used herein, for example: Cys Cysteine; Gln Glutamine; Glu Glutamic acid (Glutamate); Gly Glycine; His Histidine; Ile Isoleucine; Leu Leucine; Lys Lysine; Met Methionine; Phe Phenylalanine; Pro Proline; Ser Serine; Thr Threonine; Trp Tryptophan; Tyr Tyrosine; Val Valine;

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “biomass” refers to a material produced by growth and/or propagation of cells. Biomass may contain cells and/or intracellular contents as well as extracellular material, including, but not limited to, compounds secreted by a cell.

The term “bioreactor” or “fermenter” refers to a closed or partially closed vessel in which cells are grown and maintained. The cells may be, but are not necessarily held in liquid suspension. In some embodiments, rather than being held in liquid suspension, cells may alternatively be grown and/or maintained in contact with, on, or within another non-liquid substrate including but not limited to a solid growth support material.

“Biostimulant” or “bio-stimulant” refers to compounds capable of stimulating the growth, proliferation and/or development of cells, when provided in the culture medium, and/or to organisms, when ingested or otherwise provided to the organism in an accessible form.

The term “carbon-fixing” reaction or pathway refers to enzymatic reactions or metabolic pathways that convert C1 carbon molecules, including forms of carbon that are gaseous under ambient conditions, including but not limited to CO₂, CO, and CH₄, into carbon-based biochemicals, including biochemical molecules that are liquid or solid under ambient conditions, or which are dissolved into, or held in suspension in, aqueous solution.

“Carbon source” refers to the types of molecules from which a microorganism derives the carbon needed for organic biosynthesis.

“Carboxydotrophic” refers to microorganisms that can tolerate or oxidize carbon monoxide. In preferred embodiments a carboxydotrophic microorganism can utilize CO as a carbon source and/or as a source of reducing electrons for biosynthesis and/or respiration.

“Chemoautotrophic” refers to the ability of an organism to obtain energy by the oxidation of chemical electron donors by chemical electron acceptors and to synthesize all the organic compounds needed by the organism to live and grow from carbon dioxide.

In the claims, as well as in the specification, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

The term “culturing” refers to growing and maintaining a population of cells, e.g., microbial cells or animal cells, under suitable conditions for proliferation, propagation, maintenance, development and/or differentiation, in a liquid or solid medium.

The term “derived from” encompasses the terms “originated from,” “obtained from,” “obtainable from,” “isolated from,” and “created from,” and generally indicates that one specified material finds its origin in another specified material or has features that can be described with reference to another specified material.

“Energy source” refers to either the electron donor that is oxidized by oxygen in aerobic respiration or the combination of electron donor that is oxidized and electron acceptor that is reduced in anaerobic respiration.

“Extremophile” refers to a microorganism that thrives in physically or geochemically extreme conditions (e.g., high or low temperature, pH, or high salinity) compared to conditions on the surface of the Earth or the ocean that are typically tolerated by most life forms found on or near the earth's surface.

The term “gasification” refers to a generally high temperature process that converts carbon-based materials into a mixture of gases including hydrogen, carbon monoxide, and carbon dioxide called synthesis gas, syngas or producer gas. The process generally involves partial combustion and/or the application of externally generated heat along with the controlled addition of oxygen and/or steam such that insufficient oxygen is present for complete combustion of the carbon-based material.

“Halophile” refers to a type of extremophile that thrives in environments with very high concentrations of salt.

“Heterotrophic” refers to a mode of growth and maintenance of an organism by taking in and metabolizing organic substances, such as plant, animal or microorganism matter. Growth is heterotrophic when the organism does not synthesize all the organic compounds needed by the organism to live and grow from carbon dioxide, and utilizes organic compounds. During heterotrophic growth, organisms cannot produce their own food and instead obtain food and energy by taking in and metabolizing organic substances, such as plant or animal matter, i.e., rather than fixing carbon from inorganic sources such as carbon dioxide.

“Hydrogen-oxidizer” refers to a microorganism that utilizes reduced H₂ as an electron donor for the production of intracellular reducing equivalents and/or in respiration.

“Hyperthermophile” refers to a type of extremophile that thrives in extremely hot environments for life, typically about 60° C. (140° F.) or higher.

The term “knallgas” refers to the mixture of molecular hydrogen and oxygen gas. A “knallgas microorganism” is a microbe that can use hydrogen as an electron donor and oxygen as an electron acceptor in respiration for the generation of intracellular energy carriers such as Adenosine-5′-triphosphate (ATP). The terms “oxyhydrogen” and “oxyhydrogen microorganism” can be used synonymously with “knallgas” and “knallgas microorganism,” respectively. Knallgas microorganisms generally use molecular hydrogen by means of hydrogenases, with some of the electrons donated from H₂ that is utilized for the reduction of NAD⁺ (and/or other intracellular reducing equivalents) and some of the electrons from H₂ that is used for aerobic respiration. Knallgas microorganisms generally fix CO₂ autotrophically, through pathways including but not limited to the Calvin Cycle or the reverse citric acid cycle [“Thermophilic bacteria”, Jakob Kristjansson, Chapter 5, Section III, CRC Press, (1992)].

The term “lipid” herein refers to one or more molecules (e.g., biomolecules) that include a fatty acyl group (e.g., saturated or unsaturated acyl chains). For example, the term lipids includes oils, phospholipids, free fatty acids, monoglycerides, diglycerides, and triglycerides.

The term “lysate” refers to the liquid containing a mixture and/or a solution of cell contents that result from cell lysis. In some embodiments the lysate may be dewatered, to form a concentrated lysate, or dried to form a dry solid. In certain such embodiments, the dry lysate is in a powder form. In some embodiments, the methods described herein comprise a purification of chemicals or mixture of chemicals in a cellular lysate. In some embodiments, the methods comprise a purification of amino acids and/or protein in a cellular lysate.

The term “lysis” refers to the rupture of the plasma membrane and if present, the cell wall of a cell such that a significant amount of intracellular material escapes to the extracellular space. Lysis can be performed using electrochemical, mechanical, osmotic, thermal, or viral means. In some embodiments, the methods described herein comprise performing a lysis of cells or microorganisms as described herein in order to separate a chemical or mixture of chemicals from the contents of a bioreactor. In some embodiments, the methods comprise performing a lysis of cells or microorganisms described herein in order to separate an amino acid or mixture of amino acids and/or proteins and/or peptides from the non-proteinaceous contents of a bioreactor or cellular growth medium.

“Methanogen” refers to a microorganism that generates methane as a product of anaerobic respiration.

“Methylotroph” refers to a microorganism that can use reduced one-carbon compounds, including methanol or methane, as a carbon source and/or as an electron donor for growth.

“Methanotroph” refers to a microorganism that can metabolize methane as a carbon source and/or as an electron donor for growth.

The terms “microorganism” and “microbe” mean microscopic single celled life forms.

The term “molecule” means any distinct or distinguishable structural unit of matter comprising one or more atoms, and includes for example hydrocarbons, lipids, polypeptides and polynucleotides.

A “nutritionally fastidious” strain refers to an organism with complex or specific nutritional requirements, e.g., an organism that will grow only when specific nutrients are present.

“Oligopeptide” refers to a peptide that contains a relatively small number of amino-acid residues, for example, about 2 to about 20 amino acids.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “organic compound” refers to any gaseous, liquid, or solid chemical compound that contains carbon atoms, with the following exceptions that are considered inorganic: carbides, carbonates, simple oxides of carbon, cyanides, and allotropes of pure carbon such as diamond and graphite.

“Peptide” refers to a compound consisting of two or more amino acids linked in a chain, the carboxyl group of each acid being joined to the amino group of the next by a bond of the type R—OC—NH—R′, and may include about 2 to about 50 amino acids.

As used herein, the term “polynucleotide” refers to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms “polynucleotide,” “nucleic acid,” and “oligonucleotide” are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR.sub.2 (“amidate”), P(O)R, P(O)OR′, CO or CH.sub.2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, “polypeptide” refers to a composition comprised of amino acids and recognized as a protein by those of skill in the art. The conventional one-letter or three-letter code for amino acid residues may be used. The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also, included within the definition are, for example, polypeptides containing one or more analogs of an amino acid (including, for example, unnatural amino acids, etc.), as well as other modifications known in the art.

The term “precursor to” or “precursor of” is an intermediate towards the production of one or more of the components of a finished product.

The term “probiotic” refers to a microorganism that provides health benefits when consumed, e.g., beneficial intestinal flora.

“Producer gas” refers to a gas mixture containing various proportions of H₂, CO, and CO₂, and having heat value typically ranging between one half and one tenth that of natural gas per unit volume under standard conditions. Producer gas can be generated various ways from a variety of feedstocks, including gasification, steam reforming, or autoreforming of carbon-based feedstocks. In addition to H₂, CO, and CO₂, producer gases can contain other constituents including but not limited to methane, hydrogen sulfide, condensable gases, tars, and ash depending upon the generation process and feedstock. The proportion of N₂ in the mixture can be high or low depending whether air is used as an oxidant in the reactor or not and if the heat for the reaction is provided by direct combustion or through indirect heat exchange.

The term “producing” includes both the production of compounds intracellularly and extracellularly, including the secretion of compounds from the cell.

“Psychrophile” refers to a type of extremophile capable of growth and reproduction in cold temperatures, typically about 10° C. and lower.

The term “recombinant” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally recombinant nucleic acids, polypeptides, and cells based thereon, have been manipulated by man such that they are not identical to related nucleic acids, polypeptides, and cells found in nature. A recombinant cell may also be referred to as “engineered.”

The terms “recovered,” “isolated,” “purified,” and “separated” as used herein refer to a material (e.g., a protein, nucleic acid, or cell) that is removed from at least one component with which it is naturally associated. For example, these terms may refer to a material that is substantially or essentially free from components which normally accompany it as found in its native state, such as, for example, an intact biological system.

The phrase “substantially free” as to any given component means that such component is only present, if at all, in an amount that is a functionally insignificant amount, i.e., it does not significantly negatively impact the intended performance or function of any process or product. Typically, substantially free means less than about 1%, including less than about 0.5%, including less than about 0.1%, and also including zero percent, by weight of such component.

“Sulfur-oxidizer” refers to microorganisms that utilize reduced sulfur containing compounds including but not limited to H₂S as electron donors for the production of intracellular reducing equivalents and/or in respiration.

“Syngas” or “Synthesis gas” refers to a type of gas mixture, which like producer gas contains H₂ and CO, but which has been more specifically tailored in terms of H₂ and CO content and ratio and levels of impurities for the synthesis of a particular type of chemical product, such as but not limited to methanol or fischer-tropsch diesel. Syngas generally contains H₂, CO, and CO₂ as major components, and it can be generated through established methods including: steam reforming of methane, liquid petroleum gas, or biogas; or through gasification of any organic, flammable, carbon-based material, including but not limited to biomass, waste organic matter, various polymers, peat, and coal. The hydrogen component of syngas can be increased through the reaction of CO with steam in the water gas shift reaction, with a concomitant increase in CO₂ in the syngas mixture.

“Thermophile” refers to a type of extremophile that thrives at relatively high temperatures for life, typically about 45° C. to about 122° C.

“Titer” refers to amount of a substance produced by a microorganism per unit volume in a microbial culture. For example, biomass titer may be expressed as grams of biomass produced per liter of solution (e.g., culture medium).

A “vitamin” is a compound, e.g., organic compound, that is essential for growth and/or nutrition of an organism, typically required in small quantities in the diet or in a growth or culture medium.

A “vitamer” as used herein, refers to chemical analogs of a particular vitamin that are effective in functionally substituting for each other, and/or are effective in relieving a deficiency of the vitamin.

“Wild-type” refers to a microorganism as it occurs in nature.

“Yield” refers to amount of a product produced from a feed material relative to the total amount of the substance that would be produced if all of the feed substance were converted to product. For example, amino acid yield may be expressed as % of amino acid produced relative to a theoretical yield if 100% of the feed substance were converted to amino acid.

### Protein Hydrolysates as Nutrient Sources for Culture Media and Cultured Cells

A protein hydrolysate that is derived from a chemoautotrophic microorganism, e.g., chemoautotrophic bacterium, such as a gas (e.g., gaseous carbon and/or energy source) and/or sugar grown chemoautotrophic microorganism, e.g., oxyhydrogen microorganism (e.g., oxyhydrogen bacterium) as described herein, may be used as a nutrient source that is included in media for growth of cells in culture, such as animal cells, for example animal cells that are cultured for production of cultured (humane) meat products, e.g., cultured meat analogue products, such as, for example, chicken, pork, beef, or fish humane cultured meat products, or for culture of animal cells, such as recombinant animal cells, which express therapeutics, such as therapeutic antibodies or other proteins, or small molecules. Such a protein hydrolysate as a nutrient source may be used to replace animal serum albumin in the culture medium, thereby eliminating this media component as a potential source of animal viruses, other disease vectors, and toxins.

The protein hydrolysate from the chemoautotrophic microorganism may serve as a complete source of bioactive molecules and other nutrients for growth of the cultured cells. For example, unlike plant and yeast hydrolysates, the chemoautotrophic microorganism hydrolysate may include: all essential amino acids; essential vitamins (e.g., B1, B2, B3, B6, B12); and growth stimulants (e.g., peptides, and optionally polyhydroxyalkanoates (PHA) (e.g., polyhydroxybutyrate (PHB). In one embodiment, the chemoautotrophic microorganism source of the hydrolysate is a Cupriavidus species, such as Cupriavidus necator (e.g., C. necator strain DSM 531 or DSM 541). In some embodiments, the protein hydrolysate provides PHA (e.g., PHB) to the culture media, and is derived from a Cupriavidus species, such as Cupriavidus necator (e.g., C. necator strain DSM 531 or DSM 541).

Libraries of protein hydrolysates that are derived from different species or strains of chemoautotrophic microorganisms as described herein and that include discrete compositions for inclusion in various cell culture media are provided. Different hydrolysates, prepared as described herein, that are derived from different microorganisms or from microorganisms grown under different conditions or prepared via different hydrolysis conditions, may provide different unique nutrient profiles or promote growth of culture cells with different unique properties or metabolic or biochemical profiles or product production. In some embodiments, protein hydrolysates prepared from various chemoautotrophic species or strains contain varying amounts of components that are desirable (e.g., carbohydrates, lipids, vitamins, peptides, prebiotics, hemes, flavins) or undesirable (e.g., allergens, endotoxins) for a particular culture media composition or cultured cell to be grown in the media, for example, a hydrolysate that contains a higher concentration of one or more components that is desirable and/or a lower concentration of one or more components that is undesirable for growth of a particular cultured cell, such as growth for production of a cultured meat product, or production of a particular product, such as a therapeutic protein or small molecule, in the culture. In some embodiments, hydrolysates that are prepared from chemoautotrophic microorganisms that are grown under different growth conditions, such as, but not limited to, temperature, pH, ratios of gaseous substrate (gaseous carbon and/or energy source) ratios, and/or growth medium composition, will produce microbial (e.g., bacterial) biomass with different unique compositions (and subsequent corresponding hydrolysates with different unique compositions) that are beneficial for growth and proliferation of various cultured cells, and/or bioproduct production thereof, when included in the culture media. In some embodiments, protein hydrolysates are prepared from a chemoautotrophic microorganism (e.g., chemoautotrophic bacterium) under varying conditions to produce hydrolysates with different unique compositions. Examples of such conditions includes, but are not limited to, cell lysis conditions, varying time, temperature, and/or pH for acid, base, or enzymatic hydrolysis to prepare peptide-rich hydrolysates.

In some embodiments, peptide-rich protein hydrolysates are prepared from chemoautotrophic microorganisms as described herein by exo- or endoprotease digestion. For example, peptide and free amino acid compositions may be prepared by exoprotease (e.g., alkaline protease) hydrolysis, single sequence-specific endoprotease hydrolysis, or combinatorial or sequential hydrolysis by a sequence-specific endoprotease, of biomass of a chemoautotrophic microorganism (e.g., chemoautotrophic bacterium) or a lysate thereof.

### Methods for Producing Protein Hydrolysate Compositions

### Culturing a Microorganism

In certain embodiments, a method of the present disclosure includes culturing a microorganism, e.g., chemoautotrophic microorganism, in a bioreactor or fermenter under conditions suitable for microorganism growth and generation of a biomass that may then be converted into a protein hydrolysate composition. Any suitable methods may be used to culture the microorganisms. The microorganism may be grown under any suitable conditions, in an environment that is suitable for growth and production of biomass. In some embodiments, the microorganism may be grown in autotrophic culture conditions, heterotrophic culture conditions, or a combination of autotrophic and heterotrophic culture conditions. A heterotrophic culture may include a suitable source of carbon and energy, such as one or more sugar (e.g., glucose, fructose, sucrose, etc.). An autotrophic culture may include C1 chemicals such as carbon monoxide, carbon dioxide, methane, methanol, formate, and/or formic acid, and/or mixtures containing C1 chemicals, including, but not limited to various syngas compositions or various producer gas compositions, e.g., generated from low value sources of carbon and energy, such as, but not limited to, lignocellulosic energy crops, crop residues, bagasse, saw dust, forestry residue, or food, through the gasification, partial oxidation, pyrolysis, or steam reforming of said low value carbon sources, that can be used by an oxyhydrogen microorganism or hydrogen-oxidizing microorganism or carbon monoxide oxidizing microorganism as a carbon source and an energy source. Suitable ways of culturing the microorganisms and generating a biomass for use in the present methods are described, e.g., in PCT Application Nos. PCT/US2010/001402, PCT/US2011/034218, PCT/US2013/032362, PCT/US2014/029916, PCT/US2017/023110, PCT/US2018/016779, and U.S. Pat. No. 9,157,058, each of which is hereby incorporated by reference herein in its entirety. In some embodiments, the organism may be grown photosynthetically in a bioreactor, in a hydroponics system, in a greenhouse, or in a cultivated field, or may be collected from waste or natural sources.

The microorganism may be cultured using any suitable bioreactor or fermenter. Suitable bioreactors include but are not limited to one or more of the following: airlift reactors; biological scrubber columns; bubble columns; stirred tank reactors; continuous stirred tank reactors; counter-current, upflow, expanded-bed reactors; digesters and in particular digester systems such as known in the prior arts of sewage and waste water treatment or bioremediation; filters including but not limited to trickling filters, rotating biological contactor filters, rotating discs, soil filters; fluidized bed reactors; gas lift fermenters; immobilized cell reactors; loop reactors; membrane biofilm reactors; pachuca tanks; packed-bed reactors; plug-flow reactors; static mixers; trickle bed reactors; and/or vertical shaft bioreactors.

In some embodiments, the microorganisms are grown and maintained in a medium suitable for chemoautotrophic growth, containing gaseous carbon and energy sources, such as but not limited to syngas, producer gas, tail gas, pyrolysis gas, or H₂ and CO₂ and/or CO gas mixtures. There is no requirement for light in chemoautotrophic CO₂ fixation, and in certain embodiments there is little light or an absence of light in the growth environment.

In an exemplary but nonlimiting embodiment, a bioreactor containing nutrient medium is inoculated with production cells. Generally, there will follow a lag phase prior to the cells beginning to double. After the lag phase, the cell doubling time decreases and the culture goes into the logarithmic phase. The logarithmic phase is eventually followed by an increase of the doubling time that, while not intending to be limited by theory, is thought to result from either a mass transfer limitation, depletion of nutrients including nitrogen or mineral sources, or a rise in the concentration of inhibitory chemicals, or quorum sensing by the microbes. The growth slows down and then ceases when the culture enters the stationary phase. In certain embodiments, there is an arithmetic growth phase preceding the stationary phase. In order to harvest cell mass, the culture in certain embodiments is harvested in the logarithmic phase and/or in the arithmetic phase and/or in the stationary phase.

The growth conditions, including control of dissolved gases, such as carbon dioxide, oxygen, and/or other gases such as hydrogen, as well as other dissolved nutrients, trace elements, temperature and pH, may be controlled in the bioreactor. For certain embodiments, a protein-rich cell mass is grown to high densities and/or grown at high productivities, in liquid suspension within a bioreactor.

Nutrient media, as well as gases, can be added to the bioreactor as either a batch addition, or periodically, or in response to a detected depletion or programmed set point, or continuously over the period the culture is grown and maintained. For certain embodiments, the bioreactor at inoculation is filled with a starting batch of nutrient media and/or one or more gases at the beginning of growth, and no additional nutrient media and/or one or more gases are added after inoculation. For certain embodiments, nutrient media and/or one or more gases are added periodically after inoculation. For certain embodiments, nutrient media and/or one or more gases are added after inoculation in response to a detected depletion of nutrient and/or gas. For certain embodiments, nutrient media and/or one or more gases are added continuously after inoculation.

For certain embodiments, the added nutrient media does not contain any organic compounds, e.g., does not contain an organic carbon source such as sugar molecules or other organic molecules that may be metabolized by microorganisms as a carbon source.

In certain embodiments, a small amount of microorganism cells (i.e., an inoculum) is added to a set volume of culture medium; the culture is then incubated; and the cell mass passes through lag, exponential, deceleration, and stationary phases of growth.

In batch culture systems, the conditions (e.g., nutrient concentration, pH, etc.) under which the microorganism is cultivated generally change continuously throughout the period of growth. In certain non-limiting embodiments, to avoid the fluctuating conditions inherent in batch cultures, and to improve the overall productivity of the culture system, the microorganisms that are used for the production of protein and/or vitamins and/or other nutrients are grown in a continuous culture system called a chemostat (e.g., a bioreactor or other culture vessel to which fresh medium is continuously added, while culture liquid containing left over nutrients, metabolic end products and microorganisms are continuously removed at the same rate to keep the culture volume constant). In certain embodiments the microorganisms that are used for the production of protein and/or vitamins and/or other nutrients are grown in a continuous culture system called a turbidostat (e.g., a continuous microbiological culture device, which has feedback between the turbidity of the culture vessel and the dilution rate).

For certain embodiments, the bioreactors have mechanisms to enable mixing of the nutrient media that include, but are not limited to, one or more of the following: spinning stir bars, blades, impellers, or turbines; spinning, rocking, or turning vessels; gas lifts, sparging; recirculation of broth from the bottom of the container to the top via a recirculation conduit, flowing the broth through a loop and/or static mixers. The culture media may be mixed continuously or intermittently.

In certain embodiments the microorganism-containing nutrient medium may be removed from the bioreactor partially or completely, periodically or continuously, and in certain embodiments is replaced with fresh cell-free medium to maintain the cell culture in an exponential growth phase, and/or in an arithmetic growth phase, and/or to replenish the depleted nutrients in the growth medium, and/or to remove inhibitory waste products.

The ports that are standard in bioreactors may be utilized to deliver, or withdraw, gases, liquids, solids, and/or slurries, into and/or from the bioreactor vessel enclosing the microorganisms. Many bioreactors have multiple ports for different purposes (e.g., ports for media addition, gas addition, probes for pH and dissolved oxygen (DO), and sampling), and a given port may be used for various purposes during the course of a fermentation run. As an example, a port might be used to add nutrient media to the bioreactor at one point in time, and at another time might be used for sampling. In some embodiments, the multiple uses of a sampling port can be performed without introducing contamination or invasive species into the growth environment. A valve or other actuator enabling control of the sample flow or continuous sampling can be provided to a sampling port. For certain embodiments, the bioreactors are equipped with at least one port suitable for culture inoculation that can additionally serve other uses including the addition of media or gas. Bioreactor ports enable control of the gas composition and flow rate into the culture environment. For example, the ports can be used as gas inlets into the bioreactor through which gases are pumped.

For some embodiments, gases that may be pumped into a bioreactor include, but not are not limited to, one or more of the following: syngas, producer gas, pyrolysis gas, hydrogen gas, CO, CO₂, O₂, air, air/CO₂ mixtures, natural gas, biogas, methane, ammonia, nitrogen, noble gases, such as argon, as well as other gases. In some embodiments the CO₂ pumped into the system may come from sources including, but not limited to: CO₂ from the gasification of organic matter; CO₂ from the calcination of limestone, CaCO₃, to produce quicklime, CaO; CO₂ from methane steam reforming, such as the CO₂ byproduct from ammonia, methanol, or hydrogen production; CO₂ from combustion, incineration, or flaring; CO₂ byproduct of anaerobic or aerobic fermentation of sugar and/or any other organic carbon substrate used for fermentations; CO₂ byproduct of a methanotrophic bioprocess; CO₂ byproduct of a carboxydotrophic bioprocess; CO₂ byproduct from a heterotrophic metabolism; CO₂ from waste water treatment; CO₂ byproduct from sodium phosphate production; geologically or geothermally produced or emitted CO₂; CO₂ removed from acid gas or natural gas. In certain non-limiting embodiments, the CO₂ has been removed from an industrial flue gas, or intercepted from a geological source that would otherwise naturally emit into the atmosphere. In certain embodiments, the carbon source is CO₂ and/or bicarbonate and/or carbonate dissolved in sea water or other bodies of surface or underground water. In certain such embodiments the inorganic carbon may be introduced to the bioreactor dissolved in liquid water and/or as a solid. In certain embodiments, the carbon source is CO₂ captured from the atmosphere. In certain non-limiting embodiments, the CO₂ has been captured from a closed cabin as part of a closed-loop life support system, using equipment such as but not limited to a CO₂ removal assembly (CDRA), which is utilized, for example, on the International Space Station (ISS).

In certain non-limiting embodiments, geological features such as, but not limited to, geothermal and/or hydrothermal vents that emit high concentrations of energy sources (e.g., H₂, H₂S, CO gases) and/or carbon sources (e.g., CO₂, HCO₃⁻, CO₃²⁻) and/or other dissolved minerals may be utilized as nutrient sources for the microorganisms herein.

In certain embodiments, one or more gases in addition to carbon dioxide, or in place of carbon dioxide as an alternative carbon source, are either dissolved into solution and fed to the culture broth and/or dissolved directly into the culture broth, including but not limited to gaseous electron donors and/or carbon sources (e.g., hydrogen and/or CO and/or methane gas). In certain embodiments, input gases may include other electron donors and/or electron acceptors and/or carbon sources and/or mineral nutrients such as, but not limited to, other gas constituents and impurities of syngas (e.g., hydrocarbons); ammonia; hydrogen sulfide; and/or other sour gases; and/or O₂; and/or mineral containing particulates and ash.

Following passage through the reactor system holding microorganisms which uptake the gases, in certain embodiments the residual gases may either be recirculated back to the bioreactor, or burned for process heat, or flared, or injected underground, or released into the atmosphere. In certain embodiments herein utilizing H₂ as electron donor, H₂ may be fed to the culture vessel either by bubbling it through the culture medium, or by diffusing it through a hydrogen permeable-water impermeable membrane known in the art that interfaces with the liquid culture medium.

In certain embodiments, a C1 molecule such as but not limited to carbon dioxide, carbon monoxide, methane, methanol, formaldehyde, formate, or formic acid, and/or mixtures containing C1 molecules including but not limited to various syngas compositions generated from various gasified, pyrolyzed, or steam-reformed fixed carbon feedstocks, is utilized by the microorganism as a carbon source and is biochemically converted into longer chain organic molecules (i.e., C2 or longer and, in some embodiments, C5 or longer carbon chain molecules) under one or more of the following conditions: aerobic, microaerobic, anoxic, anaerobic, and/or facultative conditions. In some embodiments, gaseous CO₂ is utilized by the microorganism as a carbon source and is chemoautotrophically converted into longer chain organic molecules (i.e., C2 or longer and, in some embodiments, C5 or longer carbon chain molecules) under aerobic, microaerobic, anoxic, anaerobic, and/or facultative conditions. In some embodiments, H₂ is used as an electron donor and O₂ is used as an electron acceptor for the carbon fixation and conversion of the C1 carbon molecule into longer chain organic molecules. In some embodiments, H₂ is used as an electron donor and O₂ is used as an electron acceptor for chemoautotrophic carbon fixation and conversion of the CO₂ into longer chain organic molecules.

In certain embodiments, an organic carbon source is used as a source of carbon and/or reducing electrons in the cell metabolism. In certain embodiments, such growth and metabolism is heterotrophic or mixotrophic.

In certain embodiments, one or more of the following parameters are monitored and/or controlled in the bioreactor: waste product levels; pH; temperature; salinity; dissolved oxygen; dissolved carbon dioxide gas; liquid flow rates; agitation rate; gas pressure. In certain embodiments, the operating parameters affecting chemoautotrophic growth are monitored with sensors (e.g., dissolved oxygen probe or oxidation-reduction probe to gauge electron donor/acceptor concentrations), and/or are controlled either manually or automatically based upon feedback from sensors through the use of equipment including but not limited to actuating valves, pumps, and agitators. In certain embodiments, the temperature of the incoming broth as well as of incoming gases is regulated by systems such as, but not limited to, coolers, heaters, and/or heat exchangers.

In some embodiments the protein production and distribution of amino acid molecules produced by the microorganism is optimized through one or more of the following: control of bioreactor conditions, control of nutrient levels, and/or genetic modifications of the cells. In certain embodiments, pathways to amino acids, or proteins, or other nutrients, or whole cell products are controlled and optimized for the production of chemical products by maintaining specific growth conditions (e.g., levels of nitrogen, oxygen, phosphorous, sulfur, trace micronutrients such as inorganic ions, and if present any regulatory molecules that might not generally be considered a nutrient or energy source). In certain embodiments, dissolved oxygen (DO) may be optimized by maintaining the broth in aerobic, microaerobic, anoxic, anaerobic, or facultative conditions, depending upon the requirements of the microorganisms. A facultative environment is considered to include aerobic upper layers and anaerobic lower layers caused by stratification of the water column, or a spatial separation of aerobic or microaerobic regions, and anaerobic regions caused by spatial separation of regions exposed to O₂ containing gases and regions that are not exposed to O₂ containing gases.

In some embodiments, the microorganisms, e.g., chemoautotrophic microorganisms, are grown under conditions conducive to accumulation of polyhydroxyalkanoate (PHA), e.g., polyhydroxybutyrate (PHB) and/or polyhydroxyvalerate (PHV), by the microorganisms. In some embodiments, the microorganisms, e.g., chemoautotrophic microorganisms are grown under limitation of one or more nutrients, such as under nitrogen or phosphorous limitation, to cause accumulation of PHA (e.g., PHB; PHV). In some embodiments, the microorganism, such as a chemoautotrophic microorganism grown on H₂/CO₂ and/or syngas, accumulates PHA, such as PHB and/or PHV, in the cell biomass. In some embodiments, PHA (e.g., PHB; PHV) is accumulated to about 50% or more of the microorganism biomass by weight, about 60% or more, or about 70% or more by weight.

In certain embodiments, the microorganisms, e.g., chemoautotrophic microorganisms are grown under conditions that promote production of vitamins, such as, but not limited to, B vitamins, e.g., one or more of vitamin B₁, vitamin B₂, and/or vitamin B₁₂, by the microorganisms. In some embodiments, the microorganisms may be grown chemoautotrophically to produce one or more vitamins, such as vitamin B₁, vitamin B₂, and/or vitamin B₁₂.

A biomass generated by the cultured microorganisms, e.g., chemoautotrophic microorganisms, may be harvested using any suitable method, and a protein hydrolysate may then be prepared from the harvested biomass. In some embodiments, the biomass is separated from the liquid media using a suitable method. Suitable methods include, without limitation, centrifugation; flocculation; flotation; filtration using a membranous, hollow fiber, spiral wound, or ceramic filter system; vacuum filtration; tangential flow filtration; clarification; settling; hydrocyclone. In certain embodiments where the microbial cell mass may be immobilized on a matrix, it may be harvested by methods including but not limited to gravity sedimentation or filtration, and separated from the growth substrate by scraping or liquid shear forces.

Harvested microbial cells in certain embodiments can be broken open to prepare a lysate, using well known methods including but not limited to one or more of the following: ball milling, cavitation pressure, sonication, homogenization, or mechanical shearing. In some embodiments, the cells in the biomass may be lysed by one or more freeze-thaw cycles, a lytic enzyme, detergents, solvents, or antibiotics.

The harvested biomass in some embodiments may be dried in a process step or steps. Biomass drying can be performed in certain embodiments using any suitable method, including but not limited to, one or more of the following: centrifugation, drum drying, evaporation, freeze drying, heating, spray drying, vacuum drying, and/or vacuum filtration. In certain embodiments, waste heat can be used in drying the biomass. In certain embodiments, heat waste from the industrial source of flue gas used as a carbon source can be used in drying the biomass. In certain embodiments, the heat co-product from the generation of electron donors and/or C1 carbon source can be used for drying the biomass. In certain embodiments the heat co-product from gasification, methane stream reforming, autoreforming, or partial oxidation can be used for drying the biomass.

In certain embodiments, the biomass is further processed following drying, or, without a preceding drying step, in order to aid the separation and production of useful biochemicals. In certain embodiments, this additional processing involves the separation of the protein or lipid content or vitamins or nucleic acids or other targeted biochemicals from the microbial biomass. In certain embodiments, the separation of the lipids can be performed by using nonpolar or polar solvents to extract the lipids, such as, but not limited to one or more of: hexane, cyclohexane, dodecane, ethyl ether, alcohol (methanol, isopropanol, ethanol, etc.), tributyl phosphate, supercritical carbon dioxide, trioctylphosphine oxide, ammonia, secondary and tertiary amines, propane, acetone, propylene carbonate, dichloromethane, or chloroform. In certain embodiments, other useful biochemicals can be extracted using solvents, including but not limited to, one or more of: chloroform, dichloromethane, acetone, ethyl acetate, propylene carbonate, and tetrachloroethylene. In certain embodiments cell lysis is performed for the separation and production of useful biochemicals.

In some embodiments, no protein hydrolysis is performed on at least a portion of the microbial biomass, and the product is or includes a lysate of microbial cells. In some embodiments, there is no separation or drying steps applied to the lysate and/or hydrolysate, and the lysate and/or hydrolysate is a crude mixture of soluble and insoluble components. In certain such embodiments the lysate and/or hydrolysate is not clear and/or is turbid. In some embodiments there is a solid-liquid separation step applied to the lysate and/or hydrolysate, resulting in a soluble product and insoluble co-product. In certain embodiments a soluble lysate or hydrolysate product is clear and/or is not turbid. In certain embodiments, the lysate or hydrolysate is passed through ultra-filtration. In certain such embodiments, the ultra-filtration has around a 10,000 molecular weight cut off or less. In certain embodiments, a lysate and/or hydrolysate is subjected to one or more of the following downstream processes: centrifugation; plate and frame filtration; micro filtration; ultra-filtration; nano filtration; ion exchange chromatography. In certain embodiments, the lysate and/or hydrolysate is passed through a filter that includes one or more grades of carbon. In certain such embodiments the carbon removes color from the lysate and/or hydrolysate. In certain embodiments a lysate and/or hydrolysate and/or filtrate of a lysate and/or hydrolysate is passed through ion exchange chromatography, e.g., in order to lower the salt content.

In certain embodiments, a lysate and/or hydrolysate as disclosed herein is passed through sterile filtration prior to use for growth of another cell culture.

In certain embodiments, a lysate and/or hydrolysate and/or filtrate of the same, is concentrated using one or more of the following: falling film evaporator; rising film evaporator; membrane distillation, nano filtration; reverse osmosis.

In certain embodiments one or more of a lysate and/or hydrolysate and/or extract and/or concentrate and/or isolate as described herein is used in an industrial fermentation and/or dehydrated culture media and/or cell culture application. In certain embodiments a lysate and/or hydrolysate and/or peptide composition and/or amino acid composition as described herein is subjected to ultra-filtration to remove higher molecular weight materials. In certain embodiments a cell culture grown on a medium comprising the product of such ultra-filtration outperforms a cell culture grown on the unfiltered equivalent. In certain embodiments, one or more of a lysate and/or hydrolysate as described herein is used in the growth of animal cells in a culture. In certain such embodiments, the animal cells are mammalian. In certain embodiments, a cell culture is grown using a lysate and/or hydrolysate as described herein, which produces proteins and/or tissues used to form a meat-type product. In certain such embodiments, the meat-type product is produced for human consumption.

In certain embodiments, cell cultures are grown using a lysate and/or hydrolysate as described herein, which produce one or more pharmaceutical products. In certain embodiments, a lysate and/or protein hydrolysate and/or peptide composition and/or amino acid composition and/or other nutrient or co-factor produced as described herein replaces one or more animal-derived components in media used for the growth of various natural or recombinant cells, such as prokaryotic cells, for the production of nutritionals and/or bio-pharmaceuticals. In certain embodiments, such natural or recombinant prokaryotes include, but are not limited to, one or more of the following: Bacillus subtilis; Corynebacterium ammoniagenes; Pseudomonas sp.; Streptomyces lividans. In certain embodiments, the pharmaceutical products include, but are not limited to, one or more of: antibiotics such as, but not limited to, cephalosporins and cephamycins; anti-coagulants; blood factors; vaccines; polysaccharide vaccines; recombinant vaccines; recombinant proteins; antibodies; cytokines such as, but not limited to, Interleukin-11, Human granulocyte colony-stimulating factor (hG-CSF); fusion proteins; growth factors; interferons; clotting factors; hormones such as, but not limited to, human growth hormone, insulin, gonadotropin-releasing hormone, human parathyroid hormone; monoclonal antibodies; nucleic acids; therapeutic enzymes such as, but not limited to, human tissue plasminogen activator; fibrinolytic enzymes; therapeutic proteins such as, but not limited to, Transforming Growth Factor-α-Pseudomonas Exotoxin Fusion Protein (TGF-α-PE40), Human Epidermal Growth Factor (hEGF). In certain embodiments, cell cultures are grown using a lysate and/or hydrolysate as described herein, which produce a recombinant protein. In certain embodiments, a monoclonal antibody produced using medium components (e.g., a microbial lysate and/or hydrolysate) as described herein include, but are not limited to one or more of: Herceptin; Remicade, Rituxan, Synagis. In certain embodiments, a lysate or hydrolysate as described herein is used in a replacement for serum or serum derived components including fetal calf serum (FCS).

In certain embodiments, a whole cell biomass and/or lysate and/or protein hydrolysate and/or peptide composition and/or amino acid composition and/or PHB and/or hydroxybutyrate and/or vitamin and/or other nutrient or co-factor produced as described herein is fed to one or more other organisms or cells (e.g., one or more organisms or cells that are different than the microorganism from which the whole cell biomass and/or lysate and/or protein hydrolysate and/or peptide composition and/or amino acid composition and/or PHB and/or hydroxybutyrate and/or vitamin and/or other nutrient or co-factor is derived), including, but not limited to, one or more of the following: Actinomycetes, Aspergillus awamori, Aspergillus fumigates, Aspergillus nidulans, Aspergillus niger, Aspergillus oryzae, Aspergillus foetidus, Bacillus alkalophilus, Bacillus amyloliquefaciens, Bacillus brevis, Bacillus circulans, Bacillus clausii, Bacillus coagulans, Bacillus lentus, Bacillus licheniformis, Bacillus megaterium, Bacillus pumilis, Bacillus stearothermophilus, Bacillus subtilis, Bacillus thuringiensis, E. coli, E. coli strain B, E. coli strain C, E. coli strain K, E. coli strain W, Streptomyces lividans, Streptomyces murinus, Trichoderma atroviride, Trichoderma koningii, Trichoderma longibrachiatum, Trichoderma reesei, Trichoderma viride, Humicola insolens, Humicola lanuginose, Mucor miehei, Rhizomucor miehei, Rhodococcus opacus, 293 cells, 3T3 cells, BHK cells, CHO cells, COS cells, Cvl cells, HeLa cells, MDCK cells, P12 cells, VERO cells.

In certain embodiments, a whole cell biomass and/or lysate and/or protein hydrolysate and/or peptide composition and/or amino acid composition and/or PHB and/or hydroxybutyrate and/or vitamin and/or other nutrient or co-factor produced as described herein is fed to one or more other organisms or cells (e.g., one or more organisms or cells that are different than the microorganism from which the whole cell biomass and/or lysate and/or protein hydrolysate and/or peptide composition and/or amino acid composition and/or PHB and/or hydroxybutyrate and/or vitamin and/or other nutrient or co-factor is derived), including, but not limited to, members of one or more of the following genera: Aspergillus, Bacillus, Chrysosporium, Escherichia, Fusarium, Humicola, Kluyveromyces, Lactobacillus, Mucor, Myceliophtora, Neurospora, Penicillium, Phanerochaete, Pichia, Pleurotus, Pseudomonas, Rhizomucor, Rhodococcus, Saccharomyces, Schizosaccharomyces, Stenotrophamonas, Streptomyces, Trametes, Trichoderma, Yarrowia.

In certain embodiments, a whole cell biomass and/or lysate and/or protein hydrolysate and/or peptide composition and/or amino acid composition and/or PHB and/or hydroxybutyrate and/or vitamin and/or other nutrient or co-factor produced as described herein is fed to one or more other organisms or cells (e.g., one or more organisms or cells that are different than the microorganism from which the whole cell biomass and/or lysate and/or protein hydrolysate and/or peptide composition and/or amino acid composition and/or PHB and/or hydroxybutyrate and/or vitamin and/or other nutrient or co-factor is derived), that are beneficial to plants and/or are beneficial members of a plant microbiome and/or rhizosphere and/or are beneficial soil organisms, including but not limited to one or more of: Trichoderma atroviride; Azospirillum brasilense, Bradyrhizobium japonicum, and/or Mycorrhizal fungi including Arbuscular mycorrhizal fungi.

In certain embodiments, a whole cell biomass and/or lysate and/or protein hydrolysate and/or peptide composition and/or amino acid composition and/or PHB and/or hydroxybutyrate and/or vitamin and/or other nutrient or co-factor produced as described herein is fed to one or more other organisms or cells (e.g., one or more organisms or cells that are different than the microorganism from which the whole cell biomass and/or lysate and/or protein hydrolysate and/or peptide composition and/or amino acid composition and/or PHB and/or hydroxybutyrate and/or vitamin and/or other nutrient or co-factor is derived), including, but not limited to, one or more of the following: archaea cells, bacterial cells including gram-negative bacteria and/or gram-positive bacteria, filamentous fungal cells, fungus cells, insect cells, mammalian cells, animal cells, plant cells, yeast cells.

In certain embodiments, cell cultures are provided whole cell biomass and/or a lysate and/or a protein hydrolysate and/or a peptide composition and/or an amino acid composition and/or PHB and/or hydroxybutyrate and/or vitamins and/or other nutrients or co-factors produced as described herein as a nutrient source for the production of one or more microbial or chemical products such as but not limited to one or more of the following: polysaccharides, lipids, biodiesel, butanol, ethanol, propanol, isopropanol, propane, alkanes, olefins, aromatics, fatty alcohols, fatty acid esters, alcohols; 1,3-propanediol, 1,3-butadiene, 1,3-butanediol, 1,4-butanediol, 3-hydroxypropionate, 7-ADCA/cephalsporin, ε-caprolactone, γ-valerolactone, acrylate, acrylic acid, adipic acid, ascorbate, aspartate, ascorbic acid, aspartic acid, caprolactam, carotenoids, citrate, citric acid, DHA, docetaxel, erythromycin, ethylene, gamma butyrolactone, glutamate, glutamic acid, HPA, hydroxybutyrate, isopentenol, isoprene, isoprenoids, itaconate, itaconic acid, lactate, lactic acid, lanosterol, levulinic acid, lycopene, lysine, malate, malonic acid, peptides, omega-3 DHA, omega-3 EPA, omega-3 ALA, omega fatty acids, omega-7 fatty acids, omega-7 rich-oils, paclitaxel, PHA, PHB, polyketides, polyols, propylene, pyrrolidones, serine, sorbitol, statins, steroids, succinate, terephthalate, terpenes, THF, rubber, wax esters, polymers, commodity chemicals, industrial chemicals, specialty chemicals, paraffin replacements, additives, nutritional supplements, nutraceuticals, pharmaceuticals, pharmaceutical intermediates, personal care products; commercial enzymes, antibiotics, amino acids, vitamins, bioplastics, glycerol, jet fuel, diesel, gasoline, octane.

Use of chemoautotrophic microorganisms for the production of proteins, amino acids and other nutrients from gaseous feedstocks comprising H₂ and/or CO₂ and/or CO and/or CH₄ is described, for example, in PCT Application No. WO2017/165244, entitled MICROORGANISMS AND ARTIFICIAL ECOSYSTEMS FOR THE PRODUCTION OF PROTEIN, FOOD, AND USEFUL CO-PRODUCTS FROM C1 SUBSTRATES. This application is incorporated herein by reference in its entirety for all purposes.

Use of chemoautotrophic microorganisms for the production of plant, animal, and human nutrients from gaseous feedstocks comprising H₂ and/or CO₂ and/or CO and/or CH₄ is described, for example, in PCT Application No. WO2018/144965, entitled VEGAN NUTRIENTS, FERTILIZERS, BIOSTIMULANTS, AND SYSTEMS FOR ACCELERATED SOIL CARBON SEQUESTRATION, which is incorporated herein by reference in its entirety.

The production of protein hydrolysates from microbial sources is described, for example, in PCT Application No. PCT/US20/50902, filed on Sep. 15, 2020, and entitled MICROBIAL PROTEIN HYDROLYSATE COMPOSITIONS AND METHODS OF MAKING SAME, which is incorporated by reference herein in its entirety.

In certain non-limiting embodiments, lysates and/or hydrolysates as described herein are concentrated to about 25% to about 50% solids. In certain such embodiments, a drying step follows concentration to about 25% to about 50% solids. In certain embodiments the lysates and/or hydrolysates are in a concentrate form that is pumpable. In certain such embodiments, the pumpable lysate and/or hydrolysate is about 25% to about 60% solids. In other such embodiments, the pumpable lysate and/or hydrolysate is about 60% solids or more. In certain embodiments, a concentrated lysate and/or hydrolysate has a low enough water activity that it is microbially stable. In certain embodiments, a lysate and/or hydrolysate and/or filtrate and/or supernatant and/or concentrate of the same is pumped through a cartridge filter to remove any larger particles. In certain embodiments, a lysate and/or hydrolysate and/or filtrate and/or supernatant and/or concentrate of the same is dried by any suitable method, including, but not limited to, one or more of the following: spray drier; roller drum drier; lyophilization.

In certain embodiments, one or more defatting step is performed on the biomass and/or lysate and/or hydrolysate. In certain embodiments the one or more defatting steps removes or decreases the content of lipopolysaccharides (LPS) in the product. In certain embodiments, one or more filtration or ultrafiltration steps are performed on the lysate and/or hydrolysate. In certain embodiments the one or more filtration or ultrafiltration steps removes or decreases the content of LPS in the product. In certain embodiments, the ultrafiltration step has a molecular weight cut-off of 100 kilodaltons (kD) or less, or 50 kD or less, or 25 kD or less, or 20 kD or less, or 10 kD or less, or 5 kD or less. In certain embodiments, the LPS removed or decreased in one or more defatting steps and/or one or more filtration or ultrafiltration steps is an endotoxin.

Lysates and/or hydrolysates and/or peptide compositions and/or amino acid compositions produced as described herein can provide one or more of: peptides including, but not limited to, growth promoting peptides; amino acids; nucleosides, nucleotides and/or nucleic acids; carbohydrates; lipids; minerals such as, but not limited to, potassium (K), calcium (Ca), magnesium (Mg), iron (Fe), manganese (Mn), and trace minerals; vitamins; and/or other trace low molecular weight components. In certain embodiments, the peptides may function as growth and/or survival factors in a cell culture. In certain embodiments, lysates and/or hydrolysates and/or peptide compositions and/or amino acid compositions and/or extracts produced as described herein function as a source of nucleotides (RNA and DNA precursors) for another culture and/or consortium and/or microbiome, and/or the nucleotides are recycled back into the bioreactor and/or to the microorganisms which are used to produce proteins and/or biomass as disclosed herein. In certain embodiments, the nucleotides are utilized along with CO₂ carbon source in mixotrophic growth and/or production.

In certain embodiments, the lysates and/or hydrolysates and/or peptide compositions and/or amino acid compositions improve the growth rate and/or protein expression in a culture of cells that is grown on a medium that includes the lysate and/or hydrolysate and/or peptide composition and/or amino acid composition, in comparison to a medium that does not include the lysate and/or hydrolysate and/or peptide composition and/or amino acid composition. In certain embodiments, the lysates and/or hydrolysates and/or peptide compositions and/or amino acid compositions as described herein improve one or more of the following parameters in a culture that has received the composition: growth rate; productivity rate; and/or long-term viability. In certain embodiments, the lysates and/or hydrolysates and/or peptide compositions and/or amino acid compositions deliver or facilitate an adhesive, fibronectin-like activity by promoting the attachment and spreading of cells upon a substrate. In certain embodiments, the lysates and/or hydrolysates and/or peptide compositions and/or amino acid compositions improve the performance of cultures grown on a medium that includes the composition(s) through effects including, but not limited to, osmo-protectant and/or anti-apoptotic effects. In certain embodiments, protein hydrolysates and/or peptide compositions and/or amino acid compositions as described herein are used in cryopreservation solutions for animal cells. In certain embodiments, protein hydrolysates and/or peptide compositions and/or amino acid compositions as described herein are used in embryo freezing media. In certain said embodiments, the protein hydrolysate and/or peptide composition and/or amino acid composition improves cell and/or embryo survival during the freezing and/or thawing steps.

In some embodiments, the biomass may be processed to extract and/or purify a biodegradable polyester, prior, during, or following the production of a protein hydrolysate composition, including, but not limited to, a polyhydroxyalkanoate (PHA) polymer. In some embodiments, the biomass may be processed to extract a polymeric product that includes polyhydroxybutyrate (PHB). In some embodiments, the biomass may be processed to extract a polymeric product that includes polyhydroxyvalerate (PHV). The PHA or PHB or PHV polymer may be extracted from the biomass using any suitable method. In some embodiments, the PHA or PHB or PHV polymer may be extracted first by mixing the biomass with a solvent, such as one or more of chloroform, methanol, methylene chloride, 1,2-dichloroethane, dichloromethane, diethyl succinate, acetone, hexane, propylene carbonate, isopropanol and ethanol. In some embodiments, the biomass is lysed (e.g., by homogenization) before mixing with the solvent. The extraction may be performed at any suitable temperature, and may be performed at a temperature ranging from room temperature to 150° C., or more. In some embodiments, the extraction includes separating an aqueous phase and an organic phase after mixing the biomass with the solvent. The phase separation may be done using any suitable method, such as, but not limited to, centrifugation. In some embodiments, the extraction includes precipitation of PHA or PHB or PHV by, e.g., cooling the mixture, and/or adding an antisolvent (e.g., hexane) to the mixture. The extraction may include removing the solvent from the biomass-solvent mixture. In some embodiments, following the extraction, the extracted material is further purified by mixing the extracted material with a second solvent, such as hexane, in which non-polar lipids are soluble, but the PHA or PHB or PHV is insoluble. The second solvent may be removed after the mixing. Suitable methods for extracting a PHA or PHB or PHV polymer is described in, e.g., Fei, et al. (2016) “Effective recovery of poly-β-hydroxybutyrate (PHB) biopolymer from Cupriavidus necator using a novel and environmentally friendly solvent system” Biotechnol Prog. 32(3):678-85; Ujang, et al. (2009) “Recovery of Polyhydroxyalkanoates (PHAs) from Mixed Microbial Cultures by Simple Digestion and Saponification” Malaysia: University Teknology, Institute of Environmental and Water Resource Management, 8-15, which are incorporated herein by reference in their entireties.

### Preparing a Protein Hydrolysate Composition

A biomass generated from a microorganism, e.g., chemoautotrophic microorganism, culture may be hydrolyzed to produce a protein hydrolysate, according to aspects of the present disclosure. The hydrolysis may be carried out using one or more of physical, chemical, and enzymatic treatment(s) applied to the microorganism biomass. The quality of the resultant protein hydrolysate (e.g., peptone, peptides, hydrolyzed proteins), is defined by factors including the starting raw material, the hydrolyzing agents used, process parameters, and the degree of hydrolysis. The degree of hydrolysis is commonly defined by the Amino nitrogen/Total nitrogen (AN/TN) ratio. Different methods for protein digestion result in the release of amino acids and peptides to a different degree of hydrolysis (DH %). The size and amino acid composition of these released peptides may influence the growth rate and biomass yield of organisms fed the protein hydrolysate. The biological value of a particular protein hydrolysate is not only determined by the total levels of amino acids, but also their form (i.e., oligopeptides, di- and tri-peptides, or as free amino acids). Different commercial protein hydrolysates have different DH % and total protein contents.

In certain non-limiting embodiments, the microorganism biomass is hydrolyzed with at least one enzyme that is capable of hydrolyzing microbial (e.g., bacterial) proteins into free amino acids and/or short peptides. In certain embodiments, a controlled digestion of proteins produced as described herein is performed using added proteases and/or mixtures of proteases and peptidases. In certain embodiments, several commercially available proteolytic enzymes with various specificities are used to digest proteins produced as described herein. In other non-limiting embodiments, exogeneous enzymes are not used in the hydrolysis process. In certain embodiments, enzymatic hydrolysis includes hydrolyzing with a purified enzyme. In certain embodiments, enzymatic hydrolysis includes hydrolyzing with a mixture of an enzyme and a medium in which the enzyme was prepared.

In certain embodiments, enzymatic hydrolysis includes hydrolyzing with an enzyme of plant and/or animal and/or bacterial and/or archaea and/or fungal origin. In certain embodiments, enzymatic hydrolysis includes hydrolyzing with a mixture of one or more enzyme(s) of plant, animal, bacterial, archaea, and/or fungal origin. In certain embodiments, non-animal enzymes are utilized. In certain embodiments, enzymes used in enzymatic hydrolysis are not derived from animal sources (e.g., trypsin derived from porcine pancreas). In certain embodiments, proteases as well as non-proteolytic hydrolases are used, which can liberate primary components of the polymerized non-protein fraction of the raw material, e.g. microbial biomass, for example, chemoautotrophic biomass. In exemplary embodiments, bacterial cells may be hydrolyzed with one or more protease, lipase, and/or amylase. In certain embodiments, enzymatic hydrolysis includes one or more proteolytic enzyme(s) of bacterial, plant, fungal, archaeal, and/or animal origin.

In certain embodiments, the method includes use of one or more alkaline protease and/or acid protease and/or neutral protease for hydrolysis. In some embodiments, enzymatic hydrolysis includes the use of one or more exoprotease. In some embodiments, enzymatic hydrolysis includes the use of one or more endoproteases, including sequence specific endoproteases. Suitable endoproteases include, without limitation, serine proteases, aspartic proteases, metalloproteases and cysteine proteases. In certain embodiments, enzymatic hydrolysis includes hydrolyzing with at least one enzyme selected from pancreatin, papain, bromelain, ficin, bacterial protease, fungal protease, a neutral protease produced by Bacillus sp. Alcalase 2.4L, Bacillus B. licheniformis, and/or Subtilisin carlesberg, Esperase from B. lentus, Nutrase from B. amyloliquifacus, Protamex from Bacillus sp., Therolysin/therolase from B. thermoproteolyticus, Flavouzyme from Aspergillus oryzae, Aspergillus sp. Protease 2A, Protease N from B. subtilis, B. subtilis Neutrase, trypsin, chymotrypsin, keratinase, pepsin, subtilisin, and/or rennin. In some embodiments, the enzymatic hydrolysis includes hydrolyzing proteins in the biomass using multiple proteases sequentially or simultaneously.

In certain embodiments, a peptone or tryptone is made from the biomass and/or protein isolated from one or more of the microorganisms described herein. In certain embodiments, a pancreatic digest or tryptic digest is made from the biomass and/or protein isolated from one or more of the microorganisms of the present invention. In certain embodiments, recombinant proteases are utilized for hydrolytic processing. In certain embodiments, recombinant proteases provide unique cleavage specificity and/or more homogeneous hydrolysates.

Enzymatically hydrolyzing a microorganism biomass may include combining an enzyme and microorganism biomass in a suitable amount under suitable conditions. In certain embodiments the conditions of pressure, temperature, pH and time of the enzymatic hydrolysis are those in which maximum or a suitable level of enzyme activity is achieved. In certain embodiments performing enzymatic hydrolysis includes combining an enzyme and the microorganism biomass in a weight ratio ranging from about 0.1 to about 10 g of enzyme per 100 g of nitrogen content of the biomass. In another particular embodiment, the enzymatic hydrolysis is performed using a concentration of 0.05%-0.5% by volume of enzyme stock solution with an activity of around 70,000 units using the azocasein assay. In various exemplary embodiments, any suitable method may be employed to improve the efficiency of the enzymatic hydrolysis of the microorganism biomass. In certain embodiments, enzymatic hydrolysis includes combining an enzyme and the microorganism biomass and agitating the combined enzyme and microorganism biomass by any suitable method. The enzymatic treatment can be performed in any suitable device, such as a reactor with temperature control and stirring, for example.

In certain embodiments enzymes are utilized to cleave proteins at specific peptide bonds. For example, pepsin will digest at an amide linkage between amino acids in the protein where at least one of the amino acids is an aromatic amino acid (e.g., phenylalanine; tryptophan; or tyrosine). In certain embodiments pepsin is utilized to cleave amide bonds involving an aromatic amino acid.

As indicated above, hydrolysis may be carried out under any suitable conditions, however in various exemplary embodiments, hydrolysis may be carried out at a pH ranging from about 2 to about 10. In certain embodiments, enzymatic hydrolysis is performed under pH conditions falling within the range of pH about 4 to about 12. In other embodiments, hydrolysis is performed within the pH range of about 5 to about 9.5 or pH about 6 and about 8, or at about pH 7. In certain particular embodiments, the pH value is maintained constant during the enzymatic hydrolysis by adding a base, such as, for example, ammonia, ammonium hydroxide, potassium hydroxide, calcium oxide, or potassium phosphate, and in other embodiments pH is maintained by adding an acid such as phosphoric acid, sulfuric acid, or carbonic acid (i.e., CO₂). In various exemplary embodiments, hydrolysis may be carried out at a temperature of from about 15.5° C. to about 55° C. and for a period ranging from about 2 to about 120 hours. In certain embodiments, enzymatic hydrolysis is performed under temperature conditions ranging from about 10° C. to about 80° C., and in other embodiments under temperatures ranging from about 10° C. to about 65° C. or about 10° C. to about 55° C. The pH, temperature and duration used for hydrolysis may depend on the particular enzyme(s) used and the desired extent of hydrolysis (e.g., size distribution of the peptides).

In certain embodiments, performing enzymatic hydrolysis includes contacting an enzyme with the microorganism biomass in the presence of a catalyst. Any catalyst that improves the efficiency of the enzyme or enzymes may be employed, such as heterogeneous catalysts, homogeneous catalysts and/or electrocatalysts. In certain such embodiments, the catalyst includes at least one of iron, copper, cobalt, nickel, boron, magnesium, calcium and rare earth metals, such as but not limited to lanthanum. In certain embodiments, enzymatic hydrolysis includes contacting an enzyme with the microorganism biomass while electric current is applied. In certain embodiments, enzymatic hydrolysis includes treating the microorganism biomass with electrical current before and/or during enzymatically hydrolyzing the microorganism biomass. Electrical current may be applied to microorganism biomass by any suitable method and under any suitable conditions. In various exemplary embodiments, electrical current is applied in an amount of from about 2 V to about 120 V for periods of from about 1 to about 60 minutes.

Various exemplary embodiments of the methods herein may further include a pretreatment before enzymatically hydrolyzing the microorganism biomass. The efficiency of microbial protein hydrolysis using enzymes can be improved by using various pre-treatment methods. In certain embodiments, performing enzymatic hydrolysis includes treating the microorganism biomass with an acid or a base before enzymatic hydrolysis. In various exemplary embodiments, an acidic pretreatment is carried about by adjusting the pH of a suspension containing the biomass to a range falling from about 3 to about 5 using an acid such as, but not limited to, hydrochloric acid, sulfuric acid, phosphoric acid, or carbonic acid (i.e., CO₂). In certain embodiments, acidic pretreatment may be carried out at a temperature of from about 100° C. to about 130° C. for a period of from about 0.25 hours to about 10 hours. In various exemplary embodiments, a basic pretreatment is carried about by adjusting the pH of the biomass suspension to from about 9 to about 14, e.g., from about 10 to about 13, including from about 11 to about 13, using a base such as, but not limited to, sodium hydroxide, potassium hydroxide, ammonia hydroxide, calcium oxide, or ammonia. In certain embodiments, basic pretreatment may be carried out at a temperature of from about 100° C. to about 130° C. for a period of from about 0.25 hours to about 10 hours. In certain embodiments, enzymatic hydrolysis includes treating the microorganism biomass with ultrasonic vibration before and/or during enzymatic hydrolysis. In certain embodiments, enzymatic hydrolysis includes treating the microorganism biomass with supercritical water and/or supercritical carbon dioxide before enzymatic hydrolysis.

Certain embodiments include a method for obtaining a hydrolysate or organic enzyme extract through a “one-pot” process. In the context of the invention the expression “one-pot” means that the procedure is carried out without intermediate separation steps. In certain embodiments, the physical treatment of the cell broth containing the microorganism biomass occurs, without phase separation, at a super-atmospheric pressure and high temperature, where the cell broth is treated with a concentrated base prior to undergoing enzymatic hydrolysis. In certain embodiments, a biomass suspension is subjected to a type of “one-pot” process which, in certain non-limiting embodiments, includes the steps of: (a) adding a concentrated base to a microbial cell suspension to adjust the pH thereof; (b) subjecting the mixture obtained in step (a) to a super-atmospheric pressure and high temperature; and (c) subjecting the mixture obtained in step (b) to an enzymatic hydrolysis to obtain an organic enzyme extract. In certain embodiments, the alkaline treatment of step (a) uses a suitable base, such as a base selected from one or more of ammonium hydroxide, ammonia, potassium hydroxide and/or calcium hydroxide. In certain non-limiting embodiments, ammonium hydroxide at about 28% by weight is used, and in others about 10M potassium hydroxide is used.

In certain embodiments, after alkaline treatment of the broth, the physical treatment of the mixture is conducted at a super-atmospheric pressure and/or high temperature. In one particular non-limiting embodiment a pressure of about 102 kPa to about 141 kPa is applied in step (b) at an elevated temperature. In some particular embodiments a temperature of about 90° C. to about 140° C. is used in the physical treatment. In some particular embodiments, a pressure of about 102 kPa to about 141 kPa is applied at a temperature of about 90° C. to about 140° C. in step (b). This physical treatment is carried out in any suitable device selected by one skilled in the art, such as an autoclave, for instance.

In certain embodiments, enzymatic treatment is performed after the physical treatment. In certain embodiments, after the physical treatment and before the enzymatic treatment, a concentrated base and/or acid is added so that the mixture to be treated has the optimum pH value for the enzyme to be used. In certain embodiments one or more enzymes used in the enzymatic hydrolysis of step (c) is a proteolytic enzyme of bacterial, plant, fungal, archaeal, or animal origin. In a particular embodiment, the enzymatic hydrolysis of step (c) is conducted at a temperature of about 40° C. to about 70° C. and a pH of about 8 to about 11 for a period of about 2 hr to about 48 hr. In certain embodiments, a one-pot process increases process simplicity and/or decreases expense and/or is less polluting than comparable multi-pot processes.

Hydrolysis of microorganism biomass, with or without the assistance of enzymes, may be accomplished through the application of acid or alkali hydrolysis in combination with sufficient heat and pressure conditions. In certain non-limiting embodiments, hydrolyzing the microorganism biomass includes performing acid hydrolysis. In certain such embodiments, acid hydrolysis includes adjusting a pH of a composition containing the microbial cells with an acid, such as, for example, at least one agent selected from sulfuric acid, hydrochloric acid, phosphoric acid, carbonic acid, boric acid, acetic acid, propionic acid, and citric acid. In various exemplary embodiments, acid hydrolysis is carried out by adjusting pH of a microorganism biomass cream to a pH of about 0.5 to about 5. In certain such embodiments, acid hydrolysis comprises adjusting a pH of a suspension containing the microorganism biomass and heating the pH-adjusted suspension to a suitable temperature, for example, a temperature of about 30° C. to about 200° C. for a suitable period of time, for example, about 10 minutes to about 48 hours. In certain such embodiments, acid hydrolysis includes adjusting the pH of a suspension containing the microorganism biomass and heating the pH-adjusted suspension under pressure. In certain non-limiting embodiments, hydrolyzing the microbial cells includes performing alkali hydrolysis. In certain such embodiments, performing alkali hydrolysis comprises adjusting the pH of a suspension containing the microorganism biomass to a pH of about 8 to about 14. In certain such embodiments alkali hydrolysis includes adjusting the pH of a composition containing the microorganism biomass with at least one base, such as a base selected from potassium hydroxide, sodium hydroxide, calcium oxide, magnesium oxide, ammonium hydroxide, ammonia, and tripotassium phosphate. In certain such embodiments, alkali hydrolysis includes adjusting the pH of a composition containing the microorganism biomass and heating the pH-adjusted composition to a temperature of about 30° C. to about 200° C. for periods from about 10 minutes to about 48 hours. In certain such embodiments, alkali hydrolysis includes adjusting the pH of a composition containing the microorganism biomass and heating the pH-adjusted composition under pressure. In some embodiments, the alkali or acid hydrolysis is performed under elevated pressure. In certain embodiments, the suspension containing the microorganism biomass is subjected to a pressure of about 5 to about 40 psi during the alkali or acid hydrolysis. Such acid or alkali hydrolysis techniques as described herein generally yield a hydrolysate composition including soluble material and cell wall debris. In certain embodiments, said cell wall debris can be separated from the protein hydrolysate by solid-liquid separation such as centrifugation.

In certain non-limiting embodiments, hydrolyzing the microorganism biomass involves the addition of an acid or base. In certain embodiments, the acid or base is a strong acid or base, respectively. In certain embodiments, the acid includes one or more of: sulfuric acid, phosphoric acid, hydrochloric acid, and/or carbonic acid. In certain embodiments, the base includes one or more of: potassium hydroxide, ammonia, ammonium hydroxide, potassium phosphate, calcium oxide, magnesium oxide, and/or sodium hydroxide. Acid or base hydrolysis is not site specific and therefore generally results in more uniform peptide sizes and higher levels of low molecular weight (MW) peptides and/or free amino acids. In certain embodiments, a more uniform peptide size distribution and/or higher levels of low MW peptides is attained using acid or base hydrolysis. With sufficient processing time and/or intensity of the conditions applied (e.g., pH, temperature (T), pressure (P)), acid or base hydrolysis can completely release all or essentially all free amino acids (e.g., Casamino acids). In certain embodiments, the input proteins are largely or entirely converted to free amino acids via acid and/or base hydrolysis. Proteins that have been acid or base hydrolyzed all the way to free amino acids can typically contain up to 40% salts, such as NaCl. In certain embodiments, a largely or entirely salt-free free amino acid composition is produced, e.g., where the NaCl content is less than about 2%. In certain such embodiments, the total salt content may be less than about 20%, about 10%, about 5%, or about 2%. In acidic hydrolysis, certain amino acids (e.g., cysteine (Cys), tryptophan (Trp)) can be destroyed. In certain embodiments, an enzymatic and/or a pancreatic digest is utilized in order to preserve labile amino acids, such as cysteine and/or tryptophan.

In certain embodiments, a cell autolysis is performed using controlled temperature and/or osmotic changes, resulting in a cell lysate. In certain embodiments, autolysis may be triggered using one or more of: acid, base and/or heat. In certain embodiments the cell autolysis is followed by, and/or is accompanied by, protein digestion by endogenous enzymes, resulting in a cell lysate having hydrolyzed proteins and/or a protein hydrolysate and/or oligopeptides and/or free amino acids.

In certain embodiments, the starting biomass, e.g., chemoautotrophic biomass, or lysate comprises some proteins with MW≥10,000 Da. In certain such embodiments, the product of one or more protein hydrolysis steps described herein, results in fewer or no proteins or peptides with MW≥10,000 Da. In certain embodiments of the present invention, the average MW of the peptide and amino acids within the protein hydrolysate product generated by one or more protein hydrolysis steps described herein falls within ranges of about 9,000 to about 10,000 Da, or about 8,000 to about 9,000 Da, or about 7,000 to about 8,000 Da, or about 6,000 to about 7,000 Da, or about 5,000 to about 6,000 Da, or about 4,000 to about 5,000 Da, or about 3,000 to about 4,000 Da, or about 2,000 to about 3,000 Da, or about 1,000 to about 2,000 Da, or less than about 1,000 Da. In certain embodiments, the protein hydrolysate (PH) has an average MW for its peptide and amino acid content of around about 700 Da.

In certain embodiments, various hydrolyzing agents are used to design a PH having a desired range of peptide fractions and ratio of peptides to free amino acids and average MW. Hydrolysates with low molecular weight peptides are known to stimulate growth of dairy lactic acid bacteria (LAB). In certain embodiments, a PH that includes low MW peptides is produced. In certain embodiments, a PH that includes low MW peptides produced as described herein is provided to another culture such as, but not limited to, a culture that includes LAB. In certain said embodiments, the LAB culture includes one or more L. lactis strains. In certain embodiments, a PH produced as described herein contains a greater proportion of small peptides (three to seven amino acid residues) and free essential amino acids than one or more of the following types of protein hydrolysate: papain digest of casein; soy peptones; and/or whey.

In certain embodiments, a PH and/or nitrogen source is designed that includes a combination of oligopeptides supporting initial growth, and di- and tri-peptides and essential amino acids, which can be transported across the cell membrane and used for biomass synthesis with the minimal expenditure of free energy (ATP). In certain embodiments, a composition of amino acids, di- and tri-peptides is designed for the minimal ATP requirement in transporting the amino acids, di- and tri-peptides into another cell and/or organism.

In enzymatic and acid hydrolysis, certain amino acids, for example, cysteine (Cys) in casein hydrolysates, and tryptophan (Trp) in acid-digested casein peptone, are known to be destroyed. In certain embodiments, hydrolysis agents and process are selected in order to preserve sensitive amino acids, such as, but not limited to, Cys and/or Trp. In certain embodiments, a pancreatic digest of proteins is performed in order to conserve Trp.

There are several enzyme specificities that should be taken into account when designing a PH and culture medium. The biosynthesis of lactobacilli PrtB/PrtH proteinase is reported to be regulated by the peptide pool of the culture medium and repressed by a peptide-rich medium (Kenny, O., FitzGerald, R. J., O′Cuinn, G., Beresford, T., & Jordan, K. (2003) Growth phase and growth medium effects on the peptidase activities of Lactobacillus helveticus. International Dairy Journal. https://doi.org/10.1016/S0958-6946(03)00073-6). Likewise, the pepX and pepN of lactoccocci are reported to be regulated by the peptide content of the growth medium. On the other hand, the pepN amino peptidase activity of Lactobacillus helveticus CRL 1062 and pepX, pepI and pepN activities in Lactobacillus bulgaricus (Morel, F., Gilbert, C., Geourjon, C., Frot-Coutaz, J., Portalier, R., & Atlan, D. (1999) The prolyl aminopeptidase from Lactobacillus delbrueckii subsp. bulgaricus belongs to the α/β hydrolase fold family. Biochimica et Biophysica Acta—Protein Structure and Molecular Enzymology. https://doi.org/10.1016/S0167-4838(98)00264-7) are reported to be independent of the peptide content of the growth medium. In L. helveticus, enzymes such as aminopeptidases, dipeptidase, tripeptidase and endopeptidase activities are reported to be strain and species dependent. Hence, in cases where the lactobacilli strains will be applied as a cheese starter or adjunct culture, medium ingredients should be used that induce and maintain important metabolic pathways such as extracellular and intracellular proteolytic systems. These microbial traits enable acidification of milk within a predictable time and/or the release of enzymes needed for the development of flavor during cheese ripening.

In certain embodiments, where a peptide-rich medium represses important metabolic pathways such as extracellular and intracellular proteolytic systems, whole cell biomass and/or lysates and/or whole proteins (i.e., unhydrolyzed protein), produced as described herein (e.g., chemoautotrophically), are used as media components. In certain such embodiments, such a medium may be used for the growth of starter and/or adjunct cultures. In contrast to the case with some starter and adjunct cultures, the main priority in the production of probiotic strains (e.g., Lactobacillus acidophilus, Lactobacillus johnsonii, Lactobacillus reuteri) is high yield and number of viable cells, which will enable the cultures to survive freeze-drying and exposure to prolonged shelf life at room temperatures. In certain embodiments, nutrients such as a whole cell biomass and/or lysate and/or protein hydrolysate and/or peptide composition and/or amino acid composition and/or other nutrient or co-factor extracts and/or formulations including one or more of the preceding components is chosen so as to maximize one or more of the following characteristics of a culture fed said nutrients: yield; number of viable cells; cell survival of freeze-drying; and/or shelf life at room temperatures. In certain such embodiments, the culture includes probiotic strains such as, but not limited to, one or more of L. acidophilus, L. johnsonii, and/or L. reuteri.

In certain embodiments, undefined or defined starter cultures composed of several species/strains are produced. In certain embodiments, protein hydrolysates and/or other nutrients, and formulations of the same into complex media are designed to maintain reproducible performance and/or maintain the proper and/or desired ratio between strains/species throughout sub-culturing and fermentation processes. In order to achieve this, various factors are optimized in the medium formulation such as, but not limited to, correctly balancing carbon and nitrogen sources in the fermentation growth medium.

In some embodiments, the hydrolyzed biomass may undergo one or more post-hydrolysis processes, such as clarification, concentration, drying, pasteurization, and/or separation. In certain embodiments, the method described herein includes one or more further steps after hydrolysis wherein the hydrolysate is subjected to concentration to obtain a concentrated hydrolysate. The concentrated hydrolysate in certain embodiments has at least 40% by weight of dry matter, in other embodiments at least 50% by weight of dry matter, and in other embodiments about 50% to about 55% by weight of dry matter, or higher. This concentration may be achieved by any conventional method known in the art, such as, for example, by heating and using a rotary evaporator with a thermostatic bath or reverse osmosis, or any other suitable device. In some embodiments, the hydrolyzed biomass is lyophilized to remove most or substantially all of the water content.

In certain embodiments, the lysates and/or protein hydrolysates and/or peptides and/or amino acid compositions are further purified by ultrafiltration and/or by chemical means such as, but not limited to, acetone precipitation, e.g., to remove non-proteinaceous components. In certain embodiments, the lysates and/or protein hydrolysates and/or peptides and/or amino acid compositions are fractionated by size-exclusion chromatography. In certain embodiments, the fractions resulting from such fractionation are utilized as separate products and/or co-products. In certain embodiments, the lysates and/or protein hydrolysates and/or peptides and/or amino acid compositions are subjected to fine fractionation. In certain such embodiments, the most active peptide or peptides are recovered and purified. In certain embodiments, high-performance fractionation procedures are applied to lysates and/or protein hydrolysates and/or peptides and/or amino acid compositions produced as described herein, e.g., in order to obtain homogeneous peptides. In certain embodiments, the homogeneous peptides are sequenced.

In another particular embodiment, the method includes further step(s) following protein hydrolysis, wherein the protein hydrolysate obtained after hydrolysis is subjected to separation to obtain: (i) a soluble hydrolysate fraction, and (ii) a solid phase, insoluble hydrolysate fraction. This separation can be done by any suitable solid-liquid separation method, such as, for example, by filtration or centrifugation using a decanter or other suitable industrial equipment.

In certain non-limiting embodiments, a lysate and/or hydrolysate undergoes a solid-liquid separation step or steps, including but not limited to, one or more of: centrifugation; and/or filtration. In certain non-limiting embodiments, insoluble cell membrane components are separated out from the soluble fraction. In certain embodiments, the soluble and/or insoluble fraction is subjected to drying methods, including but not limited to, spray drying and/or freeze drying. In certain embodiments, the soluble fraction is used in cell culture applications. In certain embodiments, the insoluble fraction is used in nutritional applications for plants, animals, fungi, or other organisms.

In certain embodiments, in addition to including proteins, peptides, and amino acids, the lysate and/or hydrolysate and/or amino acid composition includes one or more of: vitamins; lipids; carbohydrates; nucleic acids; minerals; and/or other micronutrients.

Various embodiments of the hydrolysis methods described herein produce protein hydrolysates and/or peptide compositions and/or amino acid compositions of different levels of purity. In certain embodiments, a protein hydrolysate and/or peptide composition and/or amino acid composition produced as described herein is one or more of the following grades: technical grade; food grade; and/or non-food grade. In certain embodiments, a starter culture is produced using only food grade materials. In certain embodiments, a LAB culture and/or a probiotic culture and/or a single cell protein and/or animal cell culture such as, but not limited to, a mammalian cell culture and/or another nutrient including, but not limited to, one or more vitamins, is produced using only food grade materials. In certain embodiments, the protein hydrolysate and/or peptide composition and/or amino acid composition produced as described herein is utilized as a substitute for protein hydrolysates and/or peptide compositions and/or amino acid compositions commonly produced from meat and/or soy and/or wheat and/or dairy proteins.

In some embodiments, one or more batches of biomass from one or more different microorganisms or co-cultures or consortiums of microorganisms may be processed using multiple hydrolysis protocols to generate a library of hydrolysates having discrete compositions. In some embodiments, a library may be generated using one or more different organisms; one or more different hydrolyzing agents; one or more different sets of process parameters; one or more different degrees of hydrolysis; and one or more different post-hydrolysis processing and/or purification steps. Within the library of hydrolysates, a specific hydrolysate composition may be suitable for culturing of specific cell types and/or growth stages of cells, such as but not limited to animal cell types and/or growth stages of the animal cells.

In some embodiments, a biomass obtained from a microorganism, e.g., chemoautotrophic microorganism, culture may be processed to extract an organic polymer that the microorganism accumulates during growth. In some embodiments, a microorganism as described herein can grow on H₂/CO₂ and/or syngas, and the microorganism can accumulate polyhydroxyalkanoate (PHA), e.g., polyhydroxybutyrate (PHB) and/or polyhydroxyvalerate (PHV), to about 50% or more of the cell biomass by weight. In some embodiments, the microorganism has a native ability to direct a high flux of carbon through an acetyl-CoA metabolic intermediate, which can lead into fatty acid biosynthesis, along with a number of other synthetic pathways including PHA and/or PHB and/or PHV synthesis, as well as amino acids. In some embodiments, the microorganism exhibiting these traits is Cupriavidus necator (e.g., Cupriavidus necator DSM 531 or DSM 541) and/or Cupriavidus metallidurans (e.g., Cupriavidus metallidurans DSM 2839).

In certain embodiments, processing a biomass obtained from a chemoautotrophic microorganism includes extracting PHA and/or PHB and/or PHV from an insoluble hydrolysate fraction. In other embodiments, processing a biomass obtained from a chemoautotrophic microorganism includes recovering a PHA or PHB or PHV-rich solid from a soluble hydrolysate fraction. Any suitable method for extracting PHA or PHB or PHV may be used, as discussed above with respect to processing of a microorganism biomass to extract PHA or PHB or PHV.

In some embodiments, one or more lysates and/or hydrolysates and/or amino acid compositions as described herein are combined with other factors and/or peptides, to enrich a medium nutritionally and/or increase the stability of glutamine and/or enhance viable cell densities. It has been reported that small oligopeptides, low molecular weight substances, and free amino acids contribute positively to the nutritional value of a medium and/or the productivity of a culture grown on such a medium, for many different cell types. In certain embodiments, oligopeptides, low molecular weight substances, and free amino acids produced according as described herein contribute to the nutritional value of a medium and/or increase the productivity of a culture grown on such a medium. In certain embodiments, the oligopeptides, low molecular weight substances, and/or free amino acids may be added to the medium. In certain embodiments, the oligopeptides, low molecular weight substances, and/or free amino acids may be secreted and/or released into the medium by one or more microorganisms described herein. In some embodiments, the microorganisms include Cupriavidus necator (e.g., Cupriavidus necator DSM 531 or DSM 541) and/or Cupriavidus metallidurans (e.g., Cupriavidus metallidurans DSM 2839). In one embodiment, the microorganisms include Cupriavidus necator DSM 541.

In certain embodiments, peptides produced as described herein mitigate the presence of otherwise toxic levels of inorganic ions by providing a protective effect to a cell culture. In certain embodiments, a whole cell biomass and/or lysate and/or hydrolysate and/or peptide composition and/or amino acid composition as described herein is a source of one or more of: vitamins; fatty acids, including but not limited to, oleic, arachidonic, and/or linoleic acid; phospholipids; and/or sterols.

In certain embodiments, a whole cell biomass and/or lysate and/or hydrolysate and/or peptide composition and/or amino acid composition as described herein influences cell function and physiology in a cell culture or organism to which it has been applied.

### Microorganisms

In some embodiments, the microorganism of the present disclosure is a chemoautotrophic microorganism. In some embodiments, the microorganism of the present disclosure is able to perform mixotrophic growth and/or is a heterotrophic microorganism. In some embodiments, the microorganism of the present disclosure is a photosynthetic microorganism. In some embodiments, the microorganism of the present disclosure is an oxyhydrogen or knallgas strain, i.e., a microbe that can use hydrogen as an electron donor and oxygen as an electron acceptor in respiration for the generation of intracellular energy carriers such as adenosine-5′-triphosphate (ATP). Knallgas microorganisms generally use molecular hydrogen by means of hydrogenases, with some of the electrons donated from H₂ that is utilized for the reduction of NAD⁺ (and/or other intracellular reducing equivalents) and some of the electrons from H2 used for aerobic respiration. Knallgas microorganisms generally fix CO₂ chemoautotrophically, through pathways including but not limited to the Calvin Cycle or the reverse citric acid cycle.

In some embodiments the microorganisms, or a composition comprising microorganisms, comprises one or more of the following knallgas microorganisms: Aquifex pyrophilus, Aquifex aeolicus, or other Aquifex sp.; Cupriavidus necator or Cupriavidus metallidurans or other Cupriavidus sp.; Corynebacterium autotrophicum or other Corynebacterium sp.; Gordonia desulfuricans, Gordonia polyisoprenivorans, Gordonia rubripertincta, Gordonia hydrophobica, Gordonia westfalica, or other Gordonia sp.; Nocardia autotrophica, Nocardia opaca, or other Nocardia sp.; purple non-sulfur photosynthetic bacteria, including but not limited to, Rhodobacter sphaeroides, Rhodopseudomonas palustris, Rhodopseudomonas capsulata, Rhodopseudomonas viridis, Rhodopseudomonas sulfoviridis, Rhodopseudomonas blastica, Rhodopseudomonas spheroides, Rhodopseudomonas acidophila, or other Rhodopseudomonas sp.; Rhodobacter sp.; Rhodospirillum rubrum, or other Rhodospirillum sp.; Rhodococcus opacus or other Rhodococcus sp.; Rhizobium japonicum or other Rhizobium sp.; Thiocapsa roseopersicina or other Thiocapsa sp.; Pseudomonas facilis, Pseudomonas flava, Pseudomonas putida, Pseudomonas hydrogenovora, Pseudomonas hydrogenothermophila, Pseudomonas palleronii, Pseudomonas pseudoflava, Pseudomonas saccharophila, Pseudomonas thermophile, or other Pseudomonas sp.; Hydrogenomonas pantotropha, Hydrogenomonas eutropha, Hydrogenomonas facilis, or other Hydrogenomonas sp.; Hydrogenobacter thermophilus, Hydrogenobacter halophilus, Hydrogenobacter hydrogenophilus, or other Hydrogenobacter sp.; Hydrogenophilus islandicus or other Hydrogenophilus sp.; Hydrogenovibrio marinus or other Hydrogenovibrio sp.; Hydrogenothermus marinus or other Hydrogenothermus sp.; Helicobacter pylori or other Helicobacter sp.; Xanthobacter autotrophicus, Xanthobacter flavus, or other Xanthobacter sp.; Hydrogenophaga flava, Hydrogenophaga palleronfi, Hydrogenophaga pseudoflava, or other Hydrogenophaga sp.; Bradyrhizobium japonicum or other Bradyrhizobium sp.; Ralstonia eutropha or other Ralstonia sp.; Alcaligenes eutrophus, Alcaligenes facilis, Alcaligenes hydrogenophilus, Alcaligenes latus, Alcaligenes paradoxus, Alcaligenes ruhlandii, or other Alcaligenes sp.; Amycolata sp.; Aquaspirillum autotrophicum or other Aquaspirillum sp.; Arthrobacter strain 11/X, Arthrobacter methylotrophus, or other Arthrobacter sp.; Azospirillum lipoferum or other Azospirillum sp.; Variovorax paradoxus or other Variovorax sp.; Acidovorax facilis, or other Acidovorax sp.; Bacillus schlegelii, Bacillus tusciae, other Bacillus sp.; Calderobactenum hydrogenophilum or other Calderobactenum sp.; Derxia gummosa or other Derxia sp.; Flavobacterium autothermophilum or other Flavobacterium sp.; Microcyclus aquaticus or other Microcyclus sp.; Mycobacterium gordoniae or other Mycobacterium sp.; Paracoccus denitrificans or other Paracoccus sp.; Persephonella marina, Persephonella guaymasensis, or other Persephonella sp.; Renobacter vacuolatum or other Renobacter sp.; Seliberia carboxydohydrogena or other Seliberia sp., Streptomycetes coelicoflavus, Streptomycetes griseus, Streptomycetes xanthochromogenes, Streptomycetes thermocarboxydus, and other Streptomycetes sp.; Thermocrinis ruber or other Thermocrinis sp.; Wautersia sp.; cyanobacteria including but not limited to Anabaena oscillarioides, Anabaena spiroides, Anabaena cylindrica, or other Anabaena sp.; and Arthrospira platensis, Arthrospira maxima, or other Arthrospira sp.; green algae including but not limited to Scenedesmus obliquus or other Scenedesmus sp.; Chlamydomonas reinhardii or other Chlamydomonas sp.; Ankistrodesmus sp.; and Rhaphidium polymorphium or other Rhaphidium sp; as well as a consortium of microorganisms that includes oxyhydrogen microorganisms.

In some embodiments the microorganisms or compositions comprising the microorganisms comprise obligate and/or facultative chemoautotrophic microorganisms including one or more of the following: Acetoanaerobium sp.; Acetobacterium sp.; Acetogenium sp.; Achromobacter sp.; Acidianus sp.; Acinetobacter sp.; Actinomadura sp.; Aeromonas sp.; Alcaligenes sp.; Alcaligenes sp.; Aquaspirillum sp.; Arcobacter sp.; Aureobacterium sp.; Bacillus sp.; Beggiatoa sp.; Butyribacterium sp.; Carboxydothermus sp.; Clostridium sp.; Comamonas sp.; Dehalobacter sp.; Dehalococcoide sp.; Dehalospirillum sp.; Desulfobacterium sp.; Desulfomonile sp.; Desulfotomaculum sp.; Desulfovibrio sp.; Desulfurosarcina sp.; Ectothiorhodospira sp.; Enterobacter sp.; Eubacterium sp.; Ferroplasma sp.; Halothibacillus sp.; Hydrogenobacter sp.; Hydrogenomonas sp.; Leptospirillum sp.; Metallosphaera sp.; Methanobacterium sp.; Methanobrevibacter sp.; Methanococcus sp.; Methanococcoides sp.; Methanogenium sp.; Methanolobus sp.; Methanomicrobium sp.; Methanoplanus sp.; Methanosarcina sp.; Methanospirillum sp.; Methanothermus sp.; Methanothrix sp.; Micrococcus sp.; Nitrobacter sp.; Nitrobacteraceae sp., Nitrococcus sp., Nitrosococcus sp.; Nitrospina sp., Nitrospira sp., Nitrosolobus sp.; Nitrosomonas sp.; Nitrosospira sp.; Nitrosovibrio sp.; Nitrospina sp.; Oleomonas sp.; Paracoccus sp.; Peptostreptococcus sp.; Planctomycetes sp.; Pseudomonas sp.; Ralstonia sp.; Rhodobacter sp.; Rhodococcus sp.; Rhodocyclus sp.; Rhodomicrobium sp.; Rhodopseudomonas sp.; Rhodospirillum sp.; Shewanella sp.; Siderococcus sp.; Streptomyces sp.; Sulfobacillus sp.; Sulfolobus sp.; Thermothrix sp., Thiobacillus sp.; Thiomicrospira sp.; Thioploca sp.; Thiosphaera sp.; Thiothrix sp.; Thiovulum sp.; sulfur-oxidizers; hydrogen-oxidizers; iron-oxidizers; acetogens; and methanogens; consortiums of microorganisms that include chemoautotrophs; chemoautotrophs native to at least one of hydrothermal vents, geothermal vents, hot springs, cold seeps, underground aquifers, salt lakes, saline formations, mines, acid mine drainage, mine tailings, oil wells, refinery wastewater. coal seams, deep sub-surface; waste water and sewage treatment plants; geothermal power plants, sulfatara fields, and soils; and extremophiles selected from one or more of thermophiles, hyperthermophiles, acidophiles, halophiles, and psychrophiles.

In some embodiments the microorganisms or compositions comprising the microorganisms comprise a methanotroph and/or a methylotroph. In some embodiments the microorganism is in the genus Methylococcus. In some embodiments the microorganism is Methylococcus capsulatus. In some embodiments the microorganism is a methylotroph. In some embodiments the microorganism is in the genus Methylobacterium. In some embodiments the microorganism is drawn from one or more of the following species: Methylobacterium zatmanii; Methylobacterium extorquens; Methylobacterium chloromethanicum. In some embodiments, compositions are provided wherein the microorganism is a hydrogen-oxidizing chemoautotroph and/or a carboxydotroph and/or a methylotroph and/or methanotroph.

A number of different microorganisms have been characterized that are capable of growing on carbon monoxide as an electron donor and/or carbon source (i.e., carboxydotrophic microorganisms). In some cases, carboxydotrophic microorganisms can also use H₂ as an electron donor and/or grow mixotrophically. In some cases, the carboxydotrophic microorganisms are facultative chemolithoautotrophs [Biology of the Prokaryotes, edited by J Lengeler, G. Drews, H. Schlegel, John Wiley & Sons, Jul. 10, 2009, is incorporated herein by reference in its entirety.]. In some embodiments the microorganisms or compositions comprising the microorganisms comprise one or more of the following carboxydotrophic microorganisms: Acinetobacter sp.; Alcaligenes carboxydus or other Alcaligenes sp.; Arthrobacter sp.; Azomonas sp.; Azotobacter sp.; Bacillus schlegelii or other Bacillus sp.; Hydrogenophaga pseudoflava or other Hydrogenophaga sp.; Pseudomonas carboxydohydrogena, Pseudomonas carboxydovorans, Pseudomonas compransoris, Pseudomonas gazotropha, Pseudomonas thermocarboxydovorans, or other Pseudomonas sp.; Rhizobium japonicum or other Rhizobium sp.; and Streptomyces G26, Streptomyces thermoautotrophicus, or other Streptomyces sp. In certain embodiments, a carboxydotrophic microorganism is used. In certain embodiments, a carboxydotrophic microorganism that is capable of chemolithoautotrophy is used. In certain embodiments, a carboxydotrophic microorganism that is able to utilize H₂ as an electron donor in respiration and/or biosynthesis is used.

A microorganism, e.g., chemoautotrophic microorganism, of the present disclosure may be a naturally occurring strain, or may be genetically engineered. The microorganism may be genetically modified to express one or more proteins that have a high nutritive value for animal cells when the hydrolysate containing the protein is provided to the cells in culture, e.g., as a culture media supplement. In some embodiments, a chemoautotrophic microorganism may be genetically modified to express a polypeptide sequence containing multiple peptide subsequences that are interspersed with protease cleavage sites. Such a polypeptide sequence may be represented schematically as: H₂N—X—C-[A-C]ₙ—Y—COOH, where “H₂N” and “C001-1” represent the N- and C-terminus of the polypeptide, a respectively; “A” is a peptide subsequence; “C” is a protease cleavage site; “X” and “Y” are linker sequences that may or may not be present; and n is an integer of 1 or greater, e.g., 2 or greater, 5 or greater, 10 or greater, including 20 or greater. The peptide subsequence (A) may be designed to include a peptide sequence (A′) having a beneficial effect on animal cell growth in culture when provided in the culture medium. When the biomass obtained from the genetically modified microorganism is hydrolyzed using the appropriate protease for cutting the cleavage sites, the resulting protein hydrolysate may be enriched for the beneficial peptides.

In some embodiments, the microorganism, e.g., chemoautotrophic microorganism, may be genetically modified to disrupt expression of one or more endogenous genes involved in a biosynthetic pathway. In some embodiments, the microorganism may be genetically modified to disrupt expression of one or more gene(s) involved in the biosynthesis of a polyhydroxyalkanoate, such as polyhydroxybutyrate. Suitable gene(s) involved in the biosynthesis of a polyhydroxyalkanoate, and whose expression may be disrupted, include, without limitation, the gene encoding 3-ketothiolase, acetoacetyl-CoA reductase, and/or PHB synthase. In some embodiments, the microorganism may be genetically modified to disrupt or increase expression of one or more gene(s) involved in the biosynthesis of a vitamin, such as, but not limited to, vitamin B₁, vitamin B₂, and/or vitamin B₁₂. In some embodiments, the expression of a gene involved in the biosynthesis of vitamin B₁₂ is disrupted or increased. Expression of one or more genes may be disrupted or increased by any suitable method, e.g., by deleting or mutating all or part of the coding region or the regulatory region of the gene in the microorganism genome, or replicating the coding region or the regulatory region of the gene within the microorganism genome.

The microorganism may be genetically engineered using any suitable method. Genetic engineering of knallgas microorganisms is described, for example, in U.S. Pat. No. 9,879,290 B2, which is incorporated herein by reference in its entirety.

### Protein Hydrolysate Compositions

Provided herein is a protein hydrolysate composition derived from a microorganism, e.g., a chemoautotrophic microorganism, as described herein. The protein hydrolysate composition is suitable for use as a supplement for culture medium, for example, in animal cell culture media, e.g., as a replacement for all or a portion of serum, for example, to provide for a serum- or animal component-free cell culture media, for propagating, developing and/or differentiating animal cells, or to replace one or more animal-derived components of an animal cell culture media.

In certain embodiments, the protein hydrolysate composition has an organic content that is enriched in protein, peptides and/or free amino acids. In some embodiments, the organic content of the protein hydrolysate includes an amount of amino acids (in a protein, peptide or as free amino acids), weight by weight of the organic content, of about 10% or more, e.g., about 20% or more, about 30% or more, about 40% or more, about 50% or more, about 60% or more, about 70% or more, about 80% or more, including about 90% or more. In some embodiments, the organic content of the protein hydrolysate includes an amount of amino acids (in a protein, peptide or as free amino acids), weight by weight of the organic content, in a range of about 10% to about 98%, e.g., about 20% to about 98%, about 30% to about 98%, about 40% to about 98%, about 50% to about 95%, about 60% to about 95%, about 70% to about 95%, including about 75% to about 95%. The total organic content may be measured using any suitable method.

A protein hydrolysate composition of the present disclosure may include a distribution of polypeptide sizes. In some embodiments, the average size of polypeptides in the hydrolysate composition is about 25 kDa or smaller, e.g., about 20 kDa or smaller, about 15 kDa or smaller, about 10 kDa or smaller, about 5 kDa or smaller, about 2 kDa or smaller, about 1 kDa or smaller, including about 0.5 kDa or smaller. In some embodiments, the average size of polypeptides in the hydrolysate composition is in a range from about 0.1 kDa to about 100 kDa, e.g., from about 0.5 kDa to about 50 kDa, from about 0.1 kDa to about 10 kDa, from about 0.5 kDa to about 20 kDa, from about 1 kDa to about 10 kDa, including from about 1 kDa to about 5 kDa. In some embodiments, about 10% or more, e.g., about 20% or more, about 30% or more, about 40% or more, about 50% or more, about 60% or more, about 70% or more, about 80% or more, about 90% or more, including about 95% or more of the polypeptides by number is about 25 kDa or smaller, e.g., about 20 kDa or smaller, about 15 kDa or smaller, about 10 kDa or smaller, about 5 kDa or smaller, about 2 kDa or smaller, about 1 kDa or smaller, including about 0.5 kDa or smaller, e.g., about 0.1 kDa to about 100 kDa, e.g., from about 0.5 kDa to about 50 kDa, from about 0.1 kDa to about 10 kDa, from about 0.5 kDa to about 20 kDa, from about 1 kDa to about 10 kDa, including from about 1 kDa to about 5 kDa. In certain embodiments, the average MW of the peptides falls in the range of about 600 to about 1,100 Da.

In certain embodiments, a lysate, protein hydrolysate, peptide composition and/or amino acid composition produced as described herein contains peptides with MW falling in the range of about 600 to about 1,100 Da. In some embodiments, the range of molecular weights occurring in the lysate, protein hydrolysate, peptide composition, or amino acid composition is from about 0.1 kDa to about 100 kDa, or from about 0.1 kDa to about 10 kDa, or from about 1 kDa to about 10 kDa. In some embodiments, a small proportion of, or none of the molecular weights, are greater than about 10 kDa.

In some embodiments, the lysate and/or protein hydrolysate includes about 1% free amino acids, or about 5% free amino acids, or about 30% free amino acids, or about 40% or higher free amino acids.

In certain embodiments, a lysate, protein hydrolysate, extract, peptide composition, and/or amino acid composition produced as described herein has one or more of the following characteristics: at least about 60% total amino acid content (i.e., including amino acids polymerized within peptides and/or proteins and/or other biochemicals), or ≥about %, or ≥about 73%, or ≥about 75%, or about ≥80% total amino acids by weight; of which about 35% to about 40%, or about 30% to about 45%, or about 20% to about 50% are free amino acids; and about 10% to about 15%, or about 5% to about 20% are di- and tri-peptides; and about 40% to about 45%, or about 30% to about 50%, or about 20% to about 60% are oligopeptides of MW about 2,000 Da to about 3,000 Da.

In certain embodiments, a chemically defined medium is provided with low MW peptides produced as described herein and/or a protein hydrolysate as described herein that includes low MW peptides. In certain embodiments, the low MW peptides mostly or entirely are MW≤about 1,000 Da. In certain embodiments, the low MW peptides and/or protein hydrolysate is provided to a culture that includes, but is not limited to, one or more LAB, such as Lactobacillus, e.g., L. lactis, strains. In certain embodiments, a protein hydrolysate produced as described herein contains low MW peptides with an average MW of about 700 Da, less than about 700 Da, or about 700 Da to about 1000 Da. In certain embodiments, the protein hydrolysate, e.g., protein hydrolysate with low MW peptides as described herein, is provided to a culture comprising LAB.

In certain embodiments, a lysate, protein hydrolysate, extract, peptide composition, and/or amino acid composition produced as described herein contains peptides which are hydrophobic and/or contains basic peptide fractions with molecular masses ranging from about 600 Da to about 1,100 Da. In certain embodiments, a lysate, protein hydrolysate, extract, peptide composition, and/or amino acid composition produced as described herein contains a higher proportion and/or weight percentage of peptides which are hydrophobic and/or contains basic peptide fractions with molecular masses ranging from about 600 Da to about 1,100 Da, than does milk or milk-derived protein or protein hydrolysates. In certain embodiments, such hydrophobic and/or basic peptides with molecular masses ranging from about 600 Da to about 1,100 Da are provided to a LAB culture including, but not limited to, a culture that includes a Lactobacillus strain, for example, L. lactis.

In certain embodiments, a nutrient source such as a lysate, protein hydrolysate, extract, peptide composition, and/or amino acid composition produced as described herein contains a lower proportion and/or weight percentage of acidic phosphopeptides with MW from about 1,400 Da to about 3,200 Da than does milk or milk-derived protein or protein hydrolysates. In certain embodiments, the nutrient source comprising a relatively low content of acidic phosphopeptides with MW from about 1,400 Da to about 3,200 Da is provided to a LAB culture including but not limited to, a culture that includes a Lactobacillus strain, for example, L. lactis.

In certain embodiments, a protein hydrolysate produced as described herein, is provided to one or more strains that utilize protein hydrolysates for growth and/or production better than they can utilize whole proteins (i.e., unhydrolyzed proteins). In certain such embodiments, the said strain(s) comprise one or more LAB, such as lactobacilli.

In certain embodiments, a protein hydrolysate as described herein is better utilized by a culture than soy protein hydrolysate and/or whole soy proteins.

In certain embodiments, a protein hydrolysate produced as described herein is supplemented into a culture medium at a concentration of about 3% (w/v). In certain embodiments, the protein hydrolysate provided at a concentration of around about 3% (w/v) has an average peptide MW of about 700 Da, or about 700 Da to about 1000 Da. In certain embodiments, a nitrogen source (e.g., proteinaceous biomass, lysate, protein hydrolysate, peptide composition, and/or amino acid composition) produced as described herein is supplied to a culture in an amount from about 0.5% to about 2.5% (w/v).

In certain embodiments, a protein hydrolysate produced as described herein results in increased growth and/or titer of one or more product produced by a cell culture. In certain embodiments, the increased growth and/or titer (g/L) is observed in a LAB culture for biomass and/or lactic acid. In certain embodiments, supplementation with a protein hydrolysate produced as described herein results in higher growth and/or titer of product than supplementation with soy peptides, soy protein hydrolysate, and/or whole soy proteins. In certain embodiments, supplementation with a protein hydrolysate as described herein results in a lactic acid titer of ≥about 50 g/L, in comparison to an identical culture grown in the same culture medium without the protein hydrolysate supplement results in a lactic acid titer<about 50 g/L. In certain embodiments, supplementation with a protein hydrolysate produced as described herein results in a reduced fermentation time to reach pH 4.50 for a LAB culture, in comparison to an identical culture grown in the same culture medium without the protein hydrolysate supplement.

In certain embodiments, a lysate and/or hydrolysate and/or peptide composition and/or amino acid composition and/or extract as described herein includes peptides with two to ten amino acid residues. In certain embodiments, a lysate and/or protein hydrolysate and/or peptide composition as described herein, is primary composed of or includes peptides consisting of two to ten amino acid residues. In certain embodiments, a purified product is primarily composed of or includes peptides consisting of two to ten amino acid residues. In certain embodiments, a lysate and/or hydrolysate and/or peptide composition and/or amino acid composition and/or extract as described herein includes di- to pentapeptides (two to five amino acid residues). In certain said embodiments, the lysate and/or hydrolysate and/or peptide and/or amino acid composition and/or extract is primarily composed of or includes peptides consisting of di- to pentapeptides.

In some embodiments, the protein hydrolysate includes peptides that have a degree of polymerization (DP) that is about 50 or less, e.g., about 40 or less, about 30 or less, about 20 or less, about 10 or less, including about 5 or less, e.g., any of about 50, 40, 30, 20, 10, or 5 to about 1. In some embodiments, the protein hydrolysate includes peptides that have a DP in a range from about 1 to about 50, e.g., from about 1 to about 30, from about 1 to about 20, including from about 2 to about 10.

In some embodiments, a protein hydrolysate produced as described herein has an AN/TN ratio from about 10% to about 30%. In certain said embodiments, the protein hydrolysate with AN/TN from about 10% to about 30% is produced using enzymes. In some embodiments, a protein hydrolysate produced as described herein has an AN/TN ratio from about 20% to about 50%. In certain embodiments, the protein hydrolysate with AN/TN from about 20% to about 50% is produced as a pancreatic digest. In some embodiments, a protein hydrolysate produced as described herein has an AN/TN ratio from about 50% to about 80%. In certain said embodiments, the protein hydrolysate with AN/TN from about 50% to about 80% is produced using acid or base. In some embodiments, a whole cell product or lysate and/or protein hydrolysate produced as described herein has an AN/TN ratio less than about 10%. In some embodiments, a protein hydrolysate and/or amino acid composition produced as described herein has an AN/TN ratio greater than about 80%.

In some embodiments, the protein hydrolysate is substantially free of full-length proteins. In some embodiments, the protein hydrolysate is depleted of proteins and polypeptides, and is enriched in free amino acids. In some embodiments, the protein hydrolysate is substantially free of proteins and polypeptides. The size distribution of polypeptides may depend on the degree of hydrolysis achieved using one or more of enzymatic, chemical and physical means, as described herein.

In some embodiments, the protein hydrolysate composition is enriched for one or more peptides having a specific amino acid sequence that may have beneficial effect on cell growth and/or development. In certain embodiments, an enriched sequence may be represented by about 0.001% or more, e.g., about 0.01% or more, about 0.1% or more, about 1% or more, including about 5% or more of the total number of polypeptides in the hydrolysate. In some embodiments, an enriched sequence may be represented by a range of about 0.001% to about 5%, e.g., about 0.001% to about 1%, including about 0.01% to about 1% of the total number of polypeptides in the hydrolysate.

In some embodiments, the protein hydrolysate composition has an ash content of about 5% to about 50%, e.g., about 10% to about 40%, including about 10% to about 35%. In some embodiments, the protein hydrolysate composition has an ash content of less than or equal to about 5%. In some embodiments, the protein hydrolysate composition has a total nitrogen content from about 3% to about 20%, e.g., about 5% to about 15%, including about 5% and about 15%.

In certain embodiments, the protein hydrolysate composition includes one or more vitamin. In some embodiments, the protein hydrolysate composition includes vitamin B₁, vitamin B₂, and/or vitamin B₁₂, and/or vitamers thereof. In some embodiments, the protein hydrolysate composition includes vitamin B₁₂, and/or one or more vitamer thereof (e.g., cyanocobalamin, hydroxocobalamin, adenosylcobalamin, methylcobalamin). In certain embodiments, a concentration of vitamin B₁₂, and/or vitamers thereof, relative to the volatile organic matter in the protein hydrolysate, is about 2 μg vitamin B₁₂ and/or vitamer thereof/100 g dry volatile organic matter to about 6.5 μg/100 g volatile organic matter, or from about 6.5 μg/100 g volatile organic matter to about 13 μg/100 g volatile organic matter, or about 13 μg/100 g volatile organic matter or higher. In some embodiments, the vitamin B₁₂ and/or vitamer(s) thereof is derived from the microorganism, e.g., chemoautotrophic microorganism.

In certain embodiments, vitamins such as, but not limited to, riboflavin and/or vitamin B₁₂ (and/or vitamers thereof) are produced by a microorganism grown on protein hydrolysates and/or other nutrients produced as described herein. In certain embodiments, one or more flavor or fragrance such as, but not limited to, vanillin, is produced by a microorganism grown on protein hydrolysates and/or other nutrients produced as described herein.

In certain embodiments, a lysate and/or protein hydrolysate produced as described herein provides one or more of: nutrients; adhesion components; and/or growth factor analogues to another cell culture. In certain embodiments, a lysate and/or protein hydrolysate produced as described herein is used in a media that is free of animal-derived amino acids or protein hydrolysates. In certain embodiments, a lysate and/or protein hydrolysate produced as described herein is used in a serum-free media. In certain embodiments, a lysate and/or protein hydrolysate produced as described herein is used to reduce or replace animal derived amino acids, protein hydrolysates, and/or fetal calf/bovine serum, which is often utilized in human, animal, rodent, and/or insect cell cultures. In certain embodiments, a lysate, protein hydrolysate, and/or free amino acid composition as described herein is added to a cell culture in combination with ITS (Insulin-Transferrin-Selenium), e.g. about 1% ITS.

In certain embodiments, peptides produced as described herein are used as one or more of the following: directly as an amino acid source; indirectly as a stimulator (e.g., serum replacement); and/or for protection of cultured cells against shear stress (e.g., apoptosis).

In some embodiments, a lysate and/or protein hydrolysate and/or amino acid composition produced as described herein provides a source of vitamins to growing cells in a culture. The lysate and/or protein hydrolysate and/or amino acid composition may be a source of vitamins such as, but not limited to, vitamin B₁, vitamin B₂, and/or vitamin B₁₂, and/or vitamers thereof. In certain embodiments of the present invention the protein hydrolysate and/or amino acid composition may be a source of one or more vitamins including, but not limited to: vitamin A; beta-carotene; lutein; zeaxanthin; thiamine (B₁); riboflavin (B₂); niacin (B₃); pantothenic acid (B5); vitamin B₆; folate (B₉); vitamin B₁₂; choline; vitamin C; vitamin D; vitamin E; and/or vitamin K; and/or vitamers thereof. The protein hydrolysate and/or amino acid composition may be a source of one or more minerals including, but not limited to: calcium; iron; magnesium; manganese; phosphorus; potassium; sodium; and/or zinc.

In certain embodiments, a whole cell biomass product and/or a lysate and/or protein hydrolysate and/or peptide composition and/or amino acid composition and/or other nutrient, co-factor, or component, and/or formulation including one or more of the same, produced as described herein, is used to grow one or more other organisms which produce one or more of the following vitamins: vitamin A; beta-carotene; lutein; zeaxanthin; thiamine (B₁); riboflavin (B₂); niacin (B₃); pantothenic acid (B₅); vitamin B₆; folate (B₉); vitamin B₁₂; choline; vitamin C, vitamin D; vitamin E; and/or vitamin K; and/or vitamers thereof.

In certain embodiments, a whole cell biomass product and/or a lysate and/or protein hydrolysate and/or peptide composition and/or amino acid composition and/or other extract produced as described herein serves as a source of one or more of: amino acids; peptides, including oligopeptides; lipids; carbohydrates; polysaccharides; vitamins and/or minerals, including iron, for another cell culture.

In certain embodiments, a lysate and/or protein hydrolysate produced as described herein is water soluble.

In certain embodiments, a lysate and/or protein hydrolysate and/or peptide composition produced as described herein stimulates a more energy efficient metabolism in a cell culture fed the lysate and/or protein hydrolysate and/or peptide composition, in comparison to an equivalent free amino acid mixture containing the same or similar proportions and amounts of each amino acid in free form versus polymerized form. In certain embodiments, a cell culture fed a lysate and/or protein hydrolysate and/or peptide composition produced as described herein produces less ammonia and/or lactate and/or undergoes less glycolysis and/or glutaminolysis, in comparison to an equivalent free amino acid mixture containing the same or similar proportions and amounts of each amino acid in free form versus polymerized form

In certain embodiments, lysates and/or protein hydrolysates and/or amino acid compositions produced as described herein are derived from more than one microbial source. In certain embodiments, the microbial sources include one or more chemoautotrophic microorganism. In certain embodiments, the microbial sources is or includes a consortium of microorganisms.

In some embodiments, the protein hydrolysate composition includes a biodegradable polyester. In some embodiments, the protein hydrolysate composition includes a polyhydroxyalkanoate (PHA) polymer, such as PHB and/or PHV. In some embodiments, the PHA polymer may be represented by the formula: [COCH₂CH(R)O]ₙ, where R is C1-C5 alkyl, and n is an integer of 50 or greater. In some embodiments, the PHA oligomers are present which may be represented by the formula: [COCH₂CH(R)O]ₙ, where R is C1-C5 alkyl, and n is an integer of 50 or less. In some embodiments, free hydroxybutyrate and/or other hydroxyalkanoate (e.g., hydroxyvalerate) monomers are present. In some embodiments, the average molecular weight of the PHA polymer is about 1 kDa or more, e.g., about 10 kDa or more, about 100 kDa or more, including about 1,000 kDa or more. In some embodiments, the average molecular weight of the PHA polymer is in a range from about 1 kDa to about 10,000 kDa, e.g., about 10 kDa to about 5,000 kDa, including about 100 kDa to about 2,000 kDa. In some embodiments, the average molecular weight of the PHA oligomer is about 1 kDa or less. In certain embodiments, the PHA polymer includes a polyhydroxybutyrate (PHB), where R is CH3. In some embodiments, the PHA polymer is a copolymer, including a block copolymer. In some embodiments, the PHA (e.g., PHB and/or PHV) is derived from one or more microorganism, e.g., including chemoautotrophic microorganism(s).

The protein hydrolysate composition may include the biodegradable polyester, e.g., PHA (e.g., PHB and/or PHV), at about 1% w/w or more, e.g., about 5% w/w or more, about 10% w/w or more, about 20% w/w or more, about 30% w/w or more, about 40% w/w or more, including about 50% w/w or more. In some embodiments, the protein hydrolysate composition may include the biodegradable polyester, e.g., PHA (e.g., PHB and/or PHV), at a range of about 1% to about 90% w/w, e.g., about 5% to about 80% w/w, about 10% to about 70% w/w, about 20% to about 60% w/w, including about 30% to about 50% w/w.

In certain embodiments, vessels used to manufacture the lysates and/or protein hydrolysates and/or amino acid compositions as described herein have been adequately sanitized and validated treatments employed to eliminate potential cross-contamination from animal origin materials that might be produced using the same processing equipment. In certain embodiments, hydrolysis processes are performed at a facility where equipment used to process non-animal-derived materials is segregated from equipment used to process animal materials. In certain embodiments, hydrolysis processes are performed at a facility and/or with equipment that has only been used to process non-animal-derived materials. In certain embodiments, there has been no secondary exposure of biomass and/or lysates and/or hydrolysates and/or amino acid compositions produced as described herein to animal origin materials, such as nutrient components within the bacterial fermentation broth or animal-derived processing enzymes. In certain embodiments, there is an absence of secondary animal origin materials throughout the manufacturing process.

In certain embodiments, the biomass and/or lysates and/or protein hydrolysates and/or amino acid compositions as described herein do not have any pesticide and/or herbicide and/or fungicide residues and/or have not been exposed to any pesticides and/or herbicides and/or fungicides. In certain embodiments, the biomass and/or lysates and/or protein hydrolysates and/or amino acid compositions as described herein have not been exposed to any antibiotics such as, but not limited to, cephalosporin.

Also provided herein is cell culture media supplement, including but not limited to, an animal cell culture media supplement, which includes a nutrient source such as a lysate and/or protein hydrolysate and/or peptide composition and/or amino acid composition derived from a microorganism, e.g., chemoautotrophic microorganism, as described herein. In certain embodiments, the supplemental nutrient source complements the protein hydrolysates and/or other nutritive compounds and growth factors in a given culture medium.

In certain embodiments, the protein hydrolysate composition may be combined with a basal cell culture medium to provide a complete culture medium suitable for culturing animal cells. A basal medium typically includes amino acids, vitamins, organic and/or inorganic salts, trace elements, buffering salts and sugars. Examples of basal media include, without limitation, DMEM (Dulbecco's Modified Eagle's Media), DMEM/F12, Iscove's Modified Dulbecco's Medium (IMDM), IMDM/F12, IMDM/F12/NCTC 135, MEM (Minimum Essential Medium Eagle), Medium 199, RPM-I 1640, and RPMI 1640/DMEM/F12. The complete culture medium made by supplementing the basal medium with the present protein hydrolysate composition may support the growth and/or development of animal cells in culture without the need for any additional components or without the need for any animal-derived components, such as amino acids or protein hydrolysates derived from animals, or animal derived serum. In some embodiments, the basal medium may be supplemented with the protein hydrolysate composition and one or more additional components, such as, but not limited to, growth factors, or plant- or yeast-derived protein hydrolysates, etc. In certain embodiments, the lysate and/or protein hydrolysate and/or amino acid composition of the present invention may be used to substitute or replace nutrient components in basal media including, but not limited to, one or more of: amino acids; vitamins; organic and/or inorganic salts; trace elements; buffering salts; and/or sugars. In certain embodiments, the present lysate and/or protein hydrolysate and/or amino acid composition may be used in place of or as a substitute for a basal medium.

In certain embodiments, nutrient(s) such as a whole cell biomass product and/or a lysate and/or protein hydrolysate and/or peptide composition and/or amino acid composition and/or other nutrient, co-factor, or component, which have been produced as described herein (e.g., via chemoautotrophic biosynthesis from CO₂) may be combined with other typical medium components from other sources such as, but not limited to, amino acids, vitamins, organic and/or inorganic salts, trace elements, buffering salts and/or sugars, to provide a complex medium. In certain such embodiments, the said complex medium is a complete culture medium suitable for culturing another cell culture. In certain embodiments, nutrient(s) such as a whole cell biomass product and/or a lysate and/or protein hydrolysate and/or peptide composition and/or amino acid composition and/or other nutrient, co-factor, or component, which have been produced as described herein, are used to supplement and/or replace medium components in commonly used complex media such as, but not limited to: Elliker broth (also known as Lactobacilli Broth); Tryptone Glucose Yeast Extract (TGYE) medium; Lysogeny broth (also known as Luria-Bertani broth-LB); Seed medium; M9 medium; M9CA; Complex medium M101; YT Medium; MMBL media; Terrific broth; Super broth; Yeast Extract-Malt Extract (YEME) medium. In certain embodiments, a lysate and/or protein hydrolysate and/or extract produced as described herein is used to replace or supplement an animal, dairy, or plant derived protein hydrolysate and/or amino acid composition used in one of the aforementioned complex media, such as, but not limited to, one or more of the following: tryptone; casein (acid and/or enzyme) hydrolysate; peptone; casamino acids; and/or is used to replace or supplement yeast extract and/or malt extract. In certain embodiments, a lysate and/or protein hydrolysate and/or extract produced as described herein is used in a complex medium at a concentration ranging from about 5 g/L to about 25 g/L. In certain embodiments, a lysate and/or protein hydrolysate and/or extract produced as described herein is used in a complex medium at a concentration ranging from about 25 g/L to about 32 g/L, or from about 32 g/L to about 52 g/L, or greater than about 52 g/L. In certain embodiments, a surface response methodology (ÉRONESE, T. V, BOUCHU, A., PERLOT, P., PARK, J. W., PARK, K. N., WEBER, F. J., TRAMPER, J., RINZEMA, A., D′SOUZA, F., LALI, A., & others (1999) D. LEVISAUSKAS, V. GALVANAUSKAS, R. SIMUTIS and A. LÜBBERT 37-42. Biotechnology Techniques, 13, 937-943), is applied in order to determine the optimal concentration (g/L) of nutrient(s) produced as described herein, to be included in a complex medium, e.g., in order to maximize a desirable culture metric, such as, for example, biomass or product yield.

In certain embodiments, the lysate and/or hydrolysate and/or protein hydrolysate and/or peptide composition and/or amino acid composition as described herein may be used in the formulation of a growth medium for another culture including, but not limited to, a LAB strain. In certain embodiments, the nutrient amendments or supplements contribute to or meet the nitrogen requirements of another culture, including but not limited to, a LAB culture. In certain embodiments, the formulation may include a source of carbohydrates (e.g., lactose). In certain embodiments, the carbohydrate may be converted to an acid, such as but not limited, to lactic acid, for example, by a LAB culture, lowering pH, which may serve to preserve the product and contribute to taste and flavor of the product.

Yeast extracts, concentrated, soluble components from yeast cells, are also widely used in culture growth media. In certain embodiments, a formulation produced as described herein may comprise a lysate and/or hydrolysate and/or protein hydrolysate and/or peptide composition and/or amino acid composition and/or other nutrient or co-factor produced as described herein, and a yeast extract (e.g., in combination with yeast extract, or replacing a portion of yeast extract in the media). In other embodiments, a lysate and/or hydrolysate and/or protein hydrolysate and/or peptide composition and/or amino acid composition and/or other nutrient or co-factor produced as described herein is used to replace all yeast extract in a formulation.

In certain embodiments, supplementation of a basal formulation with a protein hydrolysate produced as described herein occurs in an amount ranging from about 100 mg/L to about 2.5 g/L. In certain embodiments, a culture medium is supplemented with a protein hydrolysate produced as described herein at a concentration ranging from about 0.25 to about 4 g/L, or from about 4 g/L to about 25 g/L, or from about 25 g/L to about 32 g/L, or from about 32 g/L to about 52 g/L, or greater than about 52 g/L.

In certain embodiments milk and/or meat hydrolysates are replaced by microbially derived hydrolysates produced as described herein. In certain embodiments, the lysate and/or protein hydrolysate and/or amino acid composition as described herein enables a reduction in the amount of animal-derived serum or an elimination of animal-derived serum, such as fetal calf serum, providing a completely serum-free medium. In certain embodiments, such replacement of milk and/or meat hydrolysates and/or reduction or elimination of serum facilitates regulatory approval. In certain embodiments, such replacement of milk and/or meat hydrolysates and/or reduction or elimination of serum obviates requirements to validate process removal of potential adventitious agents from suspect raw materials.

Protein hydrolysates are usually relatively stable and can be stored dry, e.g., at refrigerated temperatures. In certain embodiments, protein hydrolysate produced as described herein is stored dry and/or at refrigerated temperatures.

Production of dry medium by conventional ball-milling processes may be complicated by inclusion of certain protein hydrolysates, since the elevated temperatures and physical trauma can degrade labile hydrolysate constituents. In certain embodiments utilizing a lysate and/or protein hydrolysate and/or amino acid composition as described herein, a medium production process is utilized that reduces residence times, thermal denaturation and mechanical shear, such as hammer milling or fluid bed granulations.

### Methods of Use

Also provided is a method of culturing cells, e.g., eukaryotic cells, such as animal cells, or prokaryotic cells, such as lactic acid bacteria (LAB), in a culture medium supplemented with, or including a lysate and/or protein hydrolysate composition and/or peptide composition and/or amino acid composition and/or other nutrient(s) derived from a microorganism, e.g., a chemoautotrophic microorganism or consortium of microorganisms, as described herein. In certain embodiments, a lysate and/or protein hydrolysate and/or peptide composition and/or amino acid composition and/or other nutrient(s) produced as described herein provides an energy and/or carbon and/or nitrogen source for another culture. In certain embodiments, a lysate and/or protein hydrolysate and/or peptide composition and/or amino acid composition and/or other nutrient(s) produced as described herein supplements or replaces a nitrogen source used in a culture medium such as, but not limited to, one or more of the following: protein hydrolysates and/or amino acids from animal or plant sources; and/or yeast extracts.

In certain embodiments, a protein hydrolysate and/or peptide composition and/or amino acid composition and/or other nutrient or co-factor extract and/or formulation including one or more of the preceding components is designed and/or selected based on the nutritional needs of another microorganism and/or organism including, but not limited to, one or more of the following: peptides; amino acids; carbon source; nitrogen source; vitamins; minerals; growth factors; and/or other nutrients. In certain embodiments, the nutritional components and/or formulations are designed and selected to provide or optimize one or more of the following: predictable fermentation times; cell yield; cell viability; downstream processing; and/or shelf life storage.

In certain embodiments, a protein hydrolysate and/or peptide composition and/or amino acid composition and/or other nutrient or co-factor extract and/or formulation including one or more of the preceding components is designed and/or selected based on one or more of the following: Degree of hydrolysis; peptide profile; ratio between amino nitrogen (AN) and total nitrogen (TN); level of free amino acids; mineral content; cost of production; ease of recovery and/or downstream processing; price-performance ratio; and/or regulatory compliance.

In certain embodiments, a lysate and/or protein hydrolysate and/or peptide composition and/or amino acid composition and/or other nutrient or co-factor fulfill the nutritional requirements of another species/strain, by providing essential elements including, but not limited to, one or more of the following: amino acids; peptides; vitamins; minerals; nucleic acid bases; and/or other growth factors. In certain embodiments, one or more peptones, protein hydrolysates, yeast extracts, growth factors, and/or vitamins are replaced or substituted by one or more nutrients produced as described herein. In certain embodiments, a lysate and/or protein hydrolysate and/or peptide composition and/or amino acid composition and/or other nutrient, co-factor, or component including, but not limited to, one or more of lipids, polysaccharides, saccharides, PHB, PHA, PHV, nucleic acids, vitamins, and/or minerals is used to replace media components including but not limited to one or more of the following: sugars such as monosaccharides (e.g. glucose, fructose), disaccharides (e.g., lactose, sucrose, maltose), dextrins and/or maltodextrins; protein sources, such as non-fat dry milk, whey, and/or whey protein concentrates; protein hydrolysates or lysates, such as peptones, casein hydrolysates, whey protein hydrolysates, soy protein hydrolysates, meat protein hydrolysates, primatone, hydrolyzed cereal solids, and/or yeast extracts; sources of vitamins and minerals, such as yeast extracts, corn steep liquor; and/or other media components, such Tween/oleic acid, mineral salts, defoamers, and/or buffers.

In certain embodiments, a lysate and/or protein hydrolysate and/or peptide composition and/or amino acid composition and/or other nutrient, co-factor, or component including, but not limited to, one or more of the following: lipids; polysaccharides; saccharides; PHB; PHA; PHV; nucleic acids; vitamins minerals; is included in a media formulation that includes other media components including, but not limited to, one or more of the following: sugars such as monosaccharides (e.g., glucose, fructose), disaccharides (e.g., lactose, sucrose, maltose), dextrins and/or maltodextrins; protein sources such as non-fat dry milk, whey, and/or whey protein concentrates; protein hydrolysates or lysates such as peptones, casein hydrolysates, whey protein hydrolysates, soy protein hydrolysates, meat protein hydrolysates, hydrolyzed cereal solids, and/or yeast extracts; sources of vitamins and/or minerals such as yeast extract and/or corn steep liquor; and/or other media components such Tween/oleic acid, mineral salts, defoamers, and/or buffers.

In certain embodiments, one or more chemoautotrophic microorganisms are co-cultured with one or more heterotrophic organisms, wherein said chemoautotrophic microorganisms secrete nutrients such as, but not limited to, amino acids, into the culture broth, and wherein the heterotrophic organisms uptake and/or utilize the nutrients such as, but not limited to, amino acids, for growth and/or production. In certain embodiments, the chemoautotrophic microorganisms include a Cupriavidus microorganism, for example, Cupriavidus necator and/or Cupriavidus metallidurans. In certain such embodiments, the chemoautotrophic microorganisms include Cupriavidus necator microorganisms, including Cupriavidus necator DSM 531 and/or DSM 541, and/or Cupriavidus metallidurans, including Cupriavidus metallidurans DSM 2839. In certain embodiments, chemoautotrophic microorganisms are lysed and/or consumed; and/or chemoautotrophically synthesized proteins are hydrolyzed, by one or more other organisms in a co-culture.

Certain aspects of the compositions and methods described herein relate to the nutritional requirements of cell cultures and the use of protein hydrolysates and/or other extracts in fermentations and/or the growth of cell cultures. Certain aspects relate to the media used for fermentations and/or the growth of cell cultures. Certain aspects relate to the growth of lactic acid bacteria (LAB) and the media used in such growth. Certain aspects relate to the positive effects of specific protein hydrolysates on growth and survival of cell cultures, including but not limited, to cultures including LAB.

In certain embodiments, a chemically defined medium (CDM) is supplemented with low molecular weight peptides (LMWP) produced as described herein. In certain embodiments, the LMWP includes peptides ≤3,000 Da in molecular weight (MW). In certain embodiments, most or all or substantially all of the LMWP are ≤3,000 Da in MW. In certain embodiments, when the CDM supplemented with said LMWP is provided to grow another strain (e.g., a microorganism strain or species that is different than the microorganism from which the LMWP are derived), the number of cells increases at least 1.3-fold in comparison with growth of the other strain on CDM alone (i.e., without LMWP supplementation). In certain embodiments, the said other strain is a Lactobacillus strain, and in certain non-limiting embodiments, L. helveticus.

In some embodiments, the use of the protein hydrolysate composition in the culture medium allows culturing of animal cells without inclusion of some or any animal-derived components, such as animal-derived protein hydrolysates, amino acids, or serum, in the culture medium. Thus, in some embodiments, the method includes culturing the cells in a serum-free culture medium to which the present protein hydrolysate composition has been added. For example, the cells may be cultured in the medium in the absence of fetal bovine serum, horse serum, goat serum, or any other animal-derived serum, by providing a protein hydrolysate of the present disclosure in the culture medium. In some embodiments, the method includes culturing the cells in a culture medium containing the protein hydrolysate composition, without any animal-derived components, such as serum-derived albumin, transferrin, insulin, growth and/or other factors.

In certain embodiments, a lysate and/or protein hydrolysate and/or peptide composition and/or amino acid composition and/or other nutrient, co-factor, or component produced as described herein improves the strain viability of a culture over time. In certain embodiments, it helps in maintaining the redox-potential in a culture. The strain viability over time can be measured, for example, in terms of CFU/g (colony forming units per gram of culture).

In certain embodiments, a lysate and/or protein hydrolysate and/or peptide composition and/or amino acid composition and/or other nutrient or co-factor is produced in a consistent and reproducible manner, independent of region, season, or climate. In certain embodiments, variations between batches of the lysate and/or protein hydrolysate and/or peptide composition and/or amino acid composition and/or other nutrient or co-factor is less than that for a comparable animal- or plant-based hydrolysate.

In certain embodiments, a protein hydrolysate produced as described herein is used to produce one or more of: a starter culture; an adjunct culture; and/or one or more probiotic. In certain embodiments, a protein hydrolysate produced as described herein is used to grow an industrial starter culture, for example, an industrial starter culture fermentation. In certain embodiments, a lysate and/or protein hydrolysate and/or peptide composition and/or amino acid composition and/or other nutrient produced as described herein is incorporated into a formulation of a fermentation medium used in the production of a starter culture. In certain embodiments, the said industrial starter culture incorporates LAB. In certain embodiments, the starter culture is recovered from solution and converted to a concentrated frozen or freeze-dried form for storage, transport, and/or sale. In certain embodiments, one or more of the following downstream process steps are utilized with regard to the starter culture: concentration; addition of cryoprotective agents; freezing; and/or freeze-drying. In certain embodiments, probiotic strains that are grown on nutrients produced as described herein are used as components of starter cultures for fermented milks and/or as nutraceutical products for which they may be prepared in the form of free and/or encapsulated freeze-dried materials. Commercial starter culture concentrates are sometime available as “bulk sets” (Redi set) or “direct vat sets” (DVS). “Bulk sets” are used to prepare intermediate starter cultures, which are then inoculated into the production vat to prepare a final product. These cultures are available in frozen (about 70 mL) or freeze-dried form (about 5-10 g package), and are designed to inoculate 100-1,000 L of material. DVS cultures act as a direct inoculum in the final production culture. They are available as frozen or freeze-dried cultures where about 500 g of frozen culture is used to inoculate 2,500-5,000 L of material, depending upon the culture type and application. In certain embodiments, starter culture produced as described herein is prepared as a bulk set culture or a DVS culture.

In certain embodiments, a lysate and/or protein hydrolysate and/or peptide composition and/or amino acid composition and/or other nutrient produced as described herein is used to grow one or more strains that complement a primary starter culture and/or contribute to increased value of the final fermented product. In certain embodiments, a lysate and/or hydrolysate and/or peptide composition and/or amino acid composition of the present invention is added to a traditional fermentation of foods. In certain embodiments, a lysate and/or protein hydrolysate and/or peptide composition and/or amino acid composition provide nutrients for growth and/or viability to LAB and/or probiotic cultures. In certain embodiments, protein hydrolysates produced as described herein are used for the production of dairy and/or meat starter cultures. In certain embodiments, protein hydrolysates produced as described herein meet the nitrogen requirements of LAB species. In certain embodiments, a lysate and/or hydrolysate and/or peptide composition and/or amino acid composition as described herein is fed to a culture that includes bacterial species commonly used as starter and/or flavor and/or adjunct cultures. In certain embodiments, a lysate and/or hydrolysate and/or peptide composition and/or amino acid composition as described herein is fed to a culture incorporating LAB and/or other probiotics including, but not limited to, one or more of: Lactococci; Lactobacilli; Streptococci; Pediococci; and/or Bifidobacteria.

An important parameter impacting biomass quality is the composition and status of the cell wall of the organism, which determines the survival rate of cells during downstream processing. While the robustness of the cell wall may be species and even strain-dependent, it will also be influenced by the composition of the medium, growth conditions, and physiological status of cells at the time of harvesting. In certain embodiments, a lysate and/or protein hydrolysate and/or peptide composition and/or amino acid composition and/or other nutrient or co-factor produced as described herein affects one or more of the following: cell wall thickness; elongation of cells; and/or cell division.

In certain embodiments, a lysate and/or protein hydrolysate and/or peptide composition and/or amino acid composition and/or other nutrient or co-factor produced as described herein is fed to a culture incorporating one or more of the following microorganisms: Lactococcus lactis; Leuconostoc spp.; Streptococcus thermophilus; Lactobacillus spp. including, but not limited to, L. bulgaricus, L. delbrueckii ssp. bulgaricus, L. helveticus, L. casei, L. paracasei, L. acidophilus, L. johnsonii, L. reuteri, L. gallinarum, L. gasseri, L. plantarum; Pediococcus spp., including, but not limited to, Pediococcus pentosaceus, Pediococcus acidilactici; and/or Bifidobacterium spp., including, but not limited, to B. adolescentis, B. bifidum, B. lactis, B. longum, B. infantis. In certain embodiments, the microorganisms include one or more nutritionally fastidious strain. In certain embodiments, the microorganisms include one or more probiotic.

In certain embodiments, a whole cell biomass and/or lysate and/or protein hydrolysate and/or peptide composition and/or amino acid composition and/or other nutrient or co-factor produced as described herein is fed to one or more microorganisms and/or macroorganisms that are considered “generally recognized as safe” (GRAS) and/or have been traditionally (e.g., historically) used in making human food and fermentation products. In certain embodiments, a whole cell biomass and/or lysate and/or protein hydrolysate and/or peptide composition and/or amino acid composition and/or other nutrient or co-factor produced as described herein is fed to one or more other organisms (e.g., one or more organism that is different than the microorganism from which the whole cell biomass and/or lysate and/or protein hydrolysate and/or peptide composition and/or amino acid composition and/or other nutrient or co-factor is derived), including, but not limited to, one or more of the following: yeast, such as Candida humilis, Candida milleri, Debaryomyces hansenii, Kazachstania exigua (Saccharomyces exiguous), Saccharomyces cerevisiae, Saccharomyces florentinus, Torulaspora delbrueckii, Trichosporon beigelli; fungi, such as Aspergillus oryzae, Aspergillus sojae, Aspergillus luchuensis, Fusarium venenatum A3/5, Neurospora intermedia var. oncomensis, Rhizopus oligosporus, Rhizopus oryzae; bacteria such as Acetobacter aceti, Bacillus amyloliquefaciens, Bacillus subtilis, Bifidobacterium animalis (lactis), Bifidobacterium bifidum, Bifidobacterium breve, Bifidobacterium longum, Gluconacetobacter xylinus (Komagataeibacter xylinus), Lactobacillus acidophilus, Lactobacillus brevis, Lactobacillus casei, Lactobacillus delbrueckii subsp. bulgaricus, Lactobacillus fermentum, Lactobacillus helveticus, Lactobacillus hilgardii (Brevibacterium vermiforme), Lactobacillus kefiranofaciens, Lactobacillus lactis, Lactobacillus paracasei, Lactobacillus plantarum, Lactobacillus rhamnosus, Lactobacillus reuteri, Lactobacillus sakei, Lactobacillus sanfranciscensis, Lactococcus lactis (Streptococcus lactis; Streptococcus lactis subsp. diacetylactis), Leuconostoc sp., Leuconostoc carnosum, Leuconostoc cremoris, Leuconostoc mesenteroides, Pediococcus sp., Propionibacterium freudenreichii, Arthrospira (Spirulina) platensis, Streptococcus faecalis, Streptococcus thermophilus, Staphylococcus xylosus. In certain embodiments, the organisms exist in a co-culture or consortium or symbiotic culture of bacteria and yeast (SCOBY). In certain embodiments, the co-culture, consortium, or SCOBY includes one or more chemoautotrophic microbial strains.

In certain embodiments, a lysate and/or protein hydrolysate and/or peptide composition and/or amino acid composition and/or other nutrient produced as described herein is fed to a mesophilic or thermophilic dairy culture.

Microorganisms are employed in the manufacture of a wide range of fermented products. Use of microorganisms in fermenting foods dates to far earlier than knowledge of their existence. In certain embodiments, a lysate and/or protein hydrolysate and/or peptide composition and/or amino acid composition and/or other nutrient produced as described herein is fed to a culture that is used to produce one or more of the following food, drink, or feed items: fermented milk products including, but not limited to, yogurt, kefir, buttermilk; sour cream; cheeses including, but not limited, to cream cheese, soft cheeses, cheddar cheese, continental type cheeses, semi-hard cheeses, hard cheeses, Cheddar, Swiss, Gouda, Mozzarella, Emmental, Parmesan, Romano, Provolone, Jarlsberg, Leerdammer, Maasdam; fermented meats, sausages, saucisson, salami; sourdough bread; dosa; fermented vegetables and/or plant materials including, but not limited to, pickled vegetables, pickles, sauerkraut, cucumber, kimchi, tsukemono, tempeh, soy sauce, miso, fermented bean paste, red oncom, natto, olives and olive brine, cacao; fermented beverages including, but not limited to, tea, kombucha, jun, ginger beer; nutritional yeast; bread; beer; wine; mescal; colonche; tequila; cider; mirin; strawberry tree fruits juice; sugarcane juice; vinegar; nutraceuticals; probiotics, including beverages, powders, supplements, and capsules; tecuitlatl; dihe; silage.

In certain embodiments, a lysate and/or protein hydrolysate and/or peptide composition and/or amino acid composition provides amino acids and/or peptides that a LAB microorganism has a limited capacity to synthesize, or is unable to synthesize. In certain embodiments, the amino acids replace or supplement amino acids or peptides typically sourced from milk and/or from hydrolyzed proteins derived from milk, animals, or plants.

In order to use available proteins, peptides, and/or free amino acids as building blocks for synthesis of new proteins, including but not limited to, enzymes, they have to be translocated across the cell membrane into the cell. If proteins and peptides are too large to be handled by the uptake system of the cell, they have to be further hydrolyzed into smaller peptides or free amino acids. Some LAB are able to synthesize and secrete extracellular proteases, which break proteins down into peptides and amino acids, making them available for translocation. For example, L. lactis has a proteolytic system that includes a cell-envelope-located proteinase, three peptide transport systems, a set of intracellular peptidases, and nine different amino acid transport systems, which act in concert to digest milk proteins and supply the cell with essential and growth stimulating peptides and amino acids (Poolman, B., Juillard, V., Kunji, E. R. S., Hagting, A., & Konings, W. N. (1996) Casein-breakdown by Lactococcus lactis. In Lactic Acid Bacteria (pp. 303-326). Springer.; Kunji, E. R. S. (1996) The proteolytic systems of lactic acid bacteria. In Antonie van Leeuwenhoek, International Journal of General and Molecular Microbiology. https://doi.org/10.1007/BF00395933; Mierau, I., Venema, G., Kok, J., & Kunji, E. R. S. (1997) Casein and peptide degradation in lactic acid bacteria. Biotechnology and Genetic Engineering Reviews. https://doi.org/10.1080/02648725.1997.10647945). In certain embodiments, a biomass or whole cell product, or cell lysate, or other composition that includes whole proteins or large peptides produced as described herein, is provided to a microorganism that is able to synthesize and secrete one or more extracellular proteases and/or includes one or more proteolytic systems that are able hydrolyze proteins into peptides and/or amino acids. In certain embodiments, the microorganism is a Lactococcus microorganism, e.g., L. lactis. In certain embodiments, the peptides and/or amino acids are fed to a microorganism or organism that has a transport system for peptides and/or amino acids, which may or may not be the same organism as the microorganism providing the extracellular protease(s) and/or proteolytic system(s). In certain embodiments, a microorganism that is able to synthesize and secrete one or more extracellular proteases and/or has one or more proteolytic system is nonetheless fed a protein hydrolysate and/or peptide composition and/or amino acid composition in order to save the consumption of cell energy otherwise required for enzyme production, and thus improve cell growth and/or yield of products. Also, when essential amino acids are released too slowly from protein nutrients by a microorganism's extracellular protease(s) and/or proteolytic system(s) to support optimal growth and/or production, in certain embodiments, these essential amino acids are provided and/or supplemented in the form of a protein hydrolysate and/or peptide composition and/or amino acid composition and/or free amino acids, produced as described herein.

Some LAB lack proteolytic systems and require a medium with basic building blocks such as amino acids and ammonia as sources of nitrogen. In certain embodiments, a co-culture is provided that includes a first microorganism that is able to synthesize and secrete extracellular proteases and/or has a proteolytic system, a second, different microorganism which lacks proteolytic systems and/or requires a medium with basic building blocks such as amino acids and ammonia as sources of nitrogen. In certain embodiments, the first microorganism is a Lactococcus microorganism, such as L. lactis and/or the second microorganism a LAB.

LAB generally do not possess a functional tricarboxylic acid (TCA) cycle, which makes their energy generating pathways relatively inefficient. Homofermentative organisms such as Lactococcus lactis, Streptococcus thermophilus, Lactobacillus bulgaricus, Lactobacillus helveticus, and Lactobacillus acidophilus generate energy via the glycolytic (Embden-Meyerhoff-Parnas-EMP) pathway, where two moles of ATP are formed per mole of hexose consumed. Leuconostoc sp. generates energy via a heterofermentative pathway and only one mole of ATP is formed per mole of hexose consumed. Additionally, ATP may be generated by a chemi-osmotic energy process, e.g., lactate efflux. In addition, LAB generally do not produce any endogenous energy storage compounds such as glycogen, polyphosphate, and poly-β-hydroxybutyrate, except for the small amounts of the phosphoenol pyruvate pool. One exception is Bifidobacterium bifidum, which during the stationary phase forms storage compounds such as glycogen and polyphosphates. Consequently, in order to support anabolic processes and cell growth of LAB cultures, energy and other nutrient sources generally have to be supplied to the culture medium.

In certain embodiments herein, a lysate and/or protein hydrolysate and/or peptide composition and/or amino acid composition and/or other nutrient or co-factor produced as described herein is supplied to a LAB culture, which compensates for an incomplete tricarboxylic acid (TCA) cycle. In certain embodiments, a lysate and/or protein hydrolysate and/or peptide composition and/or amino acid composition and/or other nutrient or co-factor produced as described herein is supplied to a LAB culture, which saves energy in the culture that otherwise would be expended in synthesizing biochemicals (e.g., organic compounds) through anabolic pathways powered by the inefficient LAB metabolism, thus conserving and/or more efficiently utilizing carbon sources such as lactose or glucose, and/or ATP. In certain embodiments, the biochemicals synthesized through anabolic pathways include, but are not limited to, one or more of the following: amino acids; peptides; lipids; saccharides; polysaccharides; nucleic acids; vitamins; and/or co-factors.

In certain embodiments, a lysate and/or protein hydrolysate and/or peptide composition and/or amino acid composition and/or other nutrient or co-factor produced as described herein serves to replenish the phosphoenol pyruvate (PEP) pool and/or increase the concentration of PEP in the pool for a microbial culture, such as, but not limited to, a LAB culture.

In certain embodiments, a lysate and/or protein hydrolysate and/or extract produced as described herein provides one or more of the following to a culture, such as an LAB culture: cell wall ingredients, such as lipids, teichoic acid, and/or peptidoglycans; and/or RNA and/or DNA precursors, such as purine and pyrimidine bases. In certain embodiments, the provision of these supplements to the medium preserves sugar and/or ATP otherwise expended by the culture in synthesizing the biochemicals.

Lactococci are primary constituents in dairy cultures used for the production of many hard and semi-hard cheeses, and fermented milks and creams, which have lost their strong proteolytic activity characteristics, and have acquired auxotrophy to most amino acids (Bringel, F., & Hubert, J. C. (2003) Extent of genetic lesions of the arginine and pyrimidine biosynthetic pathways in Lactobacillus plantarum, L. paraplantarum, L. pentosus, and L. casei: Prevalence of CO2-dependent auxotrophs and characterization of deficient arg genes in L. plantarum. Applied and Environmental Microbiology. https://doi.org/10.1128/AEM.69.5.2674-2683.2003; Morishita, S., & Tarui, S. (1981. Lactic acidosis. Nihon Rinsho. Japanese Journal of Clinical Medicine, 39(11), 3459-3465), due to their long adaptation to milk, which is a fairly nutritious growth medium. Amino acid requirements and transport systems are known to be growth-limiting factors in Lactococci (Poolman and Konings 1988). In certain embodiments, a protein hydrolysate and/or peptide composition and/or amino acid composition produced as described herein is provided to a culture that includes a microorganism that has lost its proteolytic activity and/or acquired amino acid auxotrophy due to evolution on milk substrates. In certain embodiments, the microorganism that has lost proteolytic activity and/or has acquired amino acid auxotrophy is a Lactococci strain.

Dairy Lactococcus lactis subsp. lactis is auxotrophic for at least seven amino acids: Gln, Met, Leu, Ile, Val, Arg and His (Law et al. (1976)). L. lactis ssp. cremoris strains are reportedly even more demanding than L. lactis ssp. lactis and often require additional Tyr, Asn and Ala. In certain embodiments, amino acids including, but not limited to, one or more of the following: Gln; Met; Leu; Ile; Val; Arg; His; Tyr; Asn; and/or Ala, produced as described herein, are provided to another microorganism strain that is auxotrophic in one or more of those amino acids. In certain embodiments, an amino acid or proteinaceous supplement composition is provided to the auxotrophic strain in the form of one or more of: free amino acids; peptides; protein hydrolysate; protein; lysate; and/or whole cell biomass. In certain embodiments, the auxotrophic strain is dairy Lactococcus lactis subsp. lactis and/or L. lactis ssp. cremoris. In certain embodiments, a growth media, which has been supplemented with amino acids produced as described herein, in one or more of the following forms: free amino acids; peptides; protein hydrolysate; protein; lysate; and/or whole cell biomass, causes an increased growth rate of a microorganism strain, such as an auxotrophic strain, as described herein, compared to its growth rate on milk. In certain embodiments, the amino acid or proteinaceous supplement is provided at levels of up to about 4% (w/v), or up to about 2.5% (w/v), into a medium. In some embodiments, usage of the amino acid or proteinaceous supplement is about 0.5% (w/v) to about 2.5% (w/v). In certain embodiments, the amino acid supplement is provided at levels of greater than about 4% (w/v). In certain embodiments, the amino acid or proteinaceous supplement includes, but is not limited to, one or more of the following amino acids: Leu; Phe; and/or Glu. In certain embodiments the growth rate on the supplemented media is at least about 10%, 20%, or 40% higher than the growth rate on milk.

Proline (Pro) has been found to stimulate growth of L. lactis strains, regardless of their ability to synthesize this amino acid (Smid and Konings (1990)). While milk is abundant in Pro, this amino acid is reportedly not readily available, since some strains lack a proline transport system. Proline can only be transported into the cell by passive diffusion or by transport of proline containing peptides (Smid and Konings, supra). In certain embodiments, Pro produced as described herein, is provided to another culture (e.g., a culture of different microorganisms than the microorganism(s) from which the Pro is derived). In certain embodiments, the Pro is provided as a free amino acid and/or within Pro containing peptides and/or proteins. In certain embodiments, the culture provided the said form of Pro supplement contains one or more L. lactis strains. In certain embodiments, the microorganism strain to which Pro produced as described herein is provided exhibits a higher growth rate than an identical culture to which the Pro is not provided. In some embodiments, the microorganism strain is a L. lactis strain, for example, one or more of: Lactococcus lactis subsp. lactis and/or Lactococcus lactis subsp. cremoris strains.

Numerous chemically defined media have been developed for L. lactis. A standard synthetic medium (MCD), developed by Otto et al. (1983) and modified by Poolman and Konings (1988), supra, contains 47 components, including 18 amino acids and 14 vitamins. In certain embodiments, one or more of the amino acids and/or vitamins included in a chemical defined media for another microorganism (e.g., a microorganism that is different than the microorganism from which the amino acids and/or vitamins are derived), is produced as described herein. In certain such embodiments, the amino acid(s) and/or vitamin(s) are produced from CO₂ as the sole carbon source. In certain embodiments, the microorganism receiving the amino acid(s) and/or vitamin(s) is a Lactococcus microorganism, e.g., L. lactis. In certain embodiments, one or more amino acid is provided to a culture media, produced as described herein, wherein the omission of the amino acid(s) would result in at least about 75% lower growth rate and/or at least about 50% lower biomass yield for a culture provided the medium without inclusion of the amino acid(s).

Several Lactobacilli species are used as components of starter cultures for the production of yogurt (L. delbrueckii ssp. bulgaricus) and various types of cheeses (L. helveticus, L. paracasei), and as probiotics in fermented milks and/or nutraceuticals (L. acidophilus, L. johnsonii, L reuteri). Nutritional and nitrogen requirements vary significantly from one species to another and even between strains of the same species/sub-species. Several studies have been performed with the aim to elucidate general nutritional requirements of Lactobacillus sp. Elli, et al. (2000) and Chervaux, et al. (2000) described the nutrient requirements of 22 Lactobacillus strains using a chemically defined medium, which contains 21 amino acids and other nutrients including 60 components. In general, for optimal growth and viability, these Lactobacilli required fermentation media supplemented with abundant carbon and nitrogen sources, vitamins, micro and macronutrients, and nucleotide bases. In certain embodiments, one or more of the following: biomass; proteins; lysates; protein hydrolysates; peptide compositions; amino acid compositions; and/or other extracts, produced as described herein, are used to provide one or more of the following to a culture including one or more Lactobacillus strain(s): carbon source(s); nitrogen source(s); vitamin(s); macronutrient(s); micronutrient(s); and/or nucleotide base(s).

L. helveticus reportedly have greater amino acid requirements than most other Lactobacillus or Lactococcus strains. Morishita, et al. (1981), supra, indicated that the strain ATCC15009 is auxotrophic for 14 amino acids, four vitamins, and uracil, while strain CRL 1062 requires 13 amino acids (Hebert, et al. 2000). In certain embodiments, one or more of the following: amino acids; vitamins; and/or RNA and/or DNA bases, such as uracil, produced as described herein, are provided to a culture containing an auxotroph for one or more amino acids; vitamins; and/or nucleobases, such as, but not limited to, uracil. In certain embodiments, the auxotroph is a L. helveticus strain.

The probiotic Lactobacillus acidophilus (LA) reportedly requires the presence of Pro, Arg, Glu (Morishita, et al. (1981), supra, aromatic amino acids, and His (Hebert, et al. (2000), supra), for growth. The need for aromatic amino acids and His is reportedly connected to LA that does not possess a fully functional pentose phosphate pathway. However, LA is also reported to be greatly stimulated by almost all 18 amino acid types. In certain embodiments, amino acids produced as described herein including, but not limited to, one or more of: Pro, Arg, Glu, aromatic amino acids, and/or His, are provided to one or more other strains (e.g., a microorganism strain that is different than the microorganism from which the amino acid(s) are derived). In certain such embodiments, the strain(s) have a requirement for one or more of: Pro, Arg, Glu, aromatic amino acids, and/or His, for growth. In certain such embodiments, those amino acids (i.e., Pro, Arg, Glu, aromatic amino acids, and/or His) produced according to the present disclosure, are provided to one or more microorganisms that do not possess a fully functional pentose phosphate pathway. In certain such embodiments, the other strain(s) include an LA strain.

Even though it is not considered essential, Arg is reported to stimulate growth of Lactobacillus bulgaricus, Lactobacillus acidophilus, Lactobacillus reuteri, Pediococcus pentosaceus, and Streptococcus thermophilus strains. In certain non-limiting embodiments, Arg is provided to one or more other strains, e.g., a microorganism strain that is different than the microorganism from which Arg is produced as described herein, whose growth is stimulated by Arg. In certain embodiments, the Arg is provided to a culture that includes one or more of the following microorganisms: L. bulgaricus; L. acidophilus; L. reuteri; Pediococcus pentosaceus; and/or S. thermophilus strain(s). In certain embodiments, all 20 proteogenic amino acids that are encoded directly by triplet codons are produced as described herein, and are provided to one or more other strains (e.g., a microorganism strain(s) that is different than the microorganism from which the amino acid(s) are derived). In certain embodiments, the growth of the one or more other strains is stimulated by the provision of the one or more of the 20 amino acids produced according to the present disclosure and provided as described herein. In certain embodiments, the other strain(s) comprise a LA strain and/or a Lactobacillus reuteri strain.

Lactobacillus reuteri is known to be a particularly fastidious organism, which requires the amino acids Met, Glu, Tyr, Trp, His, Leu, Val, and Ala for growth, and which has a reduced growth rate when the other amino acids are not provided to the organism. In certain embodiments, amino acids produced as described herein, including but not limited to, one or more of: Met, Glu, Tyr, Trp, His, Leu, Val, and/or Ala, are provided to one or more other strains, e.g., a microorganism strain that is different than the microorganism from which the amino acids are produced as described herein. In certain such embodiments, the said other strain(s) have a requirement for one or more of: Met, Glu, Tyr, Trp, His, Leu, Val, and/or Ala, for growth. In certain embodiments, the said other strain(s) include Lactobacillus reuteri. In certain embodiments, additional amino acids beyond Met, Glu, Tyr, Trp, His, Leu, Val, and Ala, produced as described herein, are also provided to Lactobacillus reuteri.

In certain embodiments, all twenty of the proteinogenic amino acids that are encoded directly by triplet codons in the genetic code and are known as “standard” amino acids are produced as described herein, and are provided to one or more other strains and/or organisms e.g., a microorganism strain and/or organism that is different than the microorganism from which the amino acids are produced as described herein. In certain embodiments, all 22 of the proteinogenic (“protein-building”) amino acids are produced as described herein and are provided to one or more other strains and/or organisms, e.g., a microorganism strain and/or organism that is different than the microorganism from which the amino acids are produced as described herein. In certain embodiments, one or more of the roughly 500 known naturally occurring amino acids are produced as described herein and are provided to one or more other strains and/or organisms e.g., a microorganism strain and/or organism that is different than the microorganism from which the amino acids are produced as described herein.

Streptococcus thermophilus (ST) strains are essential components of yogurt cultures and some cheese cultures. Besides lactic acid, some strains produce exopolysaccharides (EPS), which contributes to the texture of fermented milk and can improve yield in some types of cheeses (Petersen, et al. (2000)). Growth of S. thermophilus requires a nitrogen source in the medium. Milk contains nitrogen suitable for growth of S. thermophilus. However, the natural supply of amino acids and non-protein nitrogen present in milk is insufficient to support S. thermophilus growth to high cell numbers. In certain embodiments herein, nutrient(s) such as a whole cell biomass product and/or a lysate and/or protein hydrolysate and/or peptide composition and/or amino acid composition and/or other nutrient, co-factor, or component is provided to a culture that produces a polysaccharide, such as, but not limited to EPS and/or lactic acid. In certain embodiments, the nutrient(s), when provided to S. thermophilus, result in increased production of EPS and/or larger capsules. In certain embodiments, the nutrient(s) improve the texture of fermented milk and/or improve the yield of cheese. In certain embodiments, the said nutrient(s) serve as a nitrogen source for S. thermophilus or supplemental nitrogen source for S. thermophilus. In certain embodiments, supplementation of milk with the nutrient(s), or replacement of milk with the nutrient(s), results in growth of a microorganism strain to higher cell numbers, in comparison to growth on milk alone. In certain embodiments, the strain grown to higher cell numbers is S. thermophilus.

S. thermophilus relies on cell wall associated proteinases for digesting whole proteins, or grows on protein hydrolysates as a source of amino acids, peptides, and oligopeptides. In certain embodiments, nutrients such as a whole cell biomass and/or lysate and/or whole proteins produced as described herein are provided to S. thermophilus, and its native proteinases are relied upon to the convert these nutrients to amino acids, peptides, and/or oligopeptides needed for growth and/or production of EPS and/or lactic acid.

For S. thermophilus, Gln and Glu, along with sulfur-containing amino acids, are considered to be essential amino acids. Also, the branched chain amino acid (BCAA) biosynthetic pathway is functional but insufficient to allow optimal growth of S. thermophilus in the absence of supplemental BCAAs (Garault, et al. (2000)). BCAAs are therefore usually supplemented. In certain embodiments, amino acids produced as described herein, free or bound within peptides and/or proteins, including but not limited to one or more of: Gln; Glu; sulfur-containing amino acids; and/or BCAAs, are provided to one or more other strains or organisms e.g., a microorganism strain and/or organism that is different than the microorganism from which the amino acids are produced as described herein. In certain such embodiments, the strain is S. thermophilus.

Protein hydrolysates, in combination with yeast extract, are reportedly able to provide an appropriate nitrogen source for optimal production of S. thermophilus. In certain embodiments, a protein hydrolysate produced as described herein is combined with yeast extract in a growth medium provided to a microorganism, such as, but not limited to S. thermophilus. In certain embodiments, yeast extract is replaced with a lysate, hydrolysate, and/or other extract produced as described herein in a medium provided to a microorganism, such as, but not limited to S. thermophilus. In certain embodiments, one or more components of Elliker broth (also known as Lactobacilli Broth) are replaced with whole cell biomass and/or a lysate and/or protein hydrolysate and/or peptide composition and/or amino acid composition and/or other nutrient(s) and/or co-factor(s) produced as described herein, resulting in a modified Elliker Broth. In certain embodiments, the components replaced in the modified Elliker Broth include, but are not limited to, casein hydrolysate and/or yeast extract. In certain embodiments, Elliker broth is supplemented with whole cell biomass and/or lysate and/or protein hydrolysate and/or peptide composition and/or amino acid composition and/or other nutrient(s) and/or co-factor(s) produced as described herein, resulting in a supplemented Elliker Broth. In certain embodiments the modified and/or supplemented Elliker Broth is used for cultivating Streptococci and/or Lactobacilli.

In certain embodiments, a protein hydrolysate produced as described herein is provided to a culture medium in at a concentration of about 5 g/L to about 20 g/L or about 5 g/L to about 25 g/L, or about 20 g/L to about 25 g/L. Addition of hydrolysates of casein and whey to milk are reported to enhance the growth and acidification rate of S. thermophilus ST-7, thus reducing the fermentation time of yogurt (Lucas, et al.). In certain embodiments, a hydrolysate produced as described herein enhances the growth and/or acidification rate of another culture. In certain embodiments, the culture comprises or consists of S. thermophilus ST-7. Addition of 2% of acid casein hydrolysate and cysteine to milk was reported to improve the viability of the S. thermophilus WJ7 over 12 weeks, when tested in frozen dairy dessert (Ravula and Shah, 1998). In certain embodiments, a protein hydrolysate produced as described herein is added to a culture medium at about 2% (w/v) concentration. In certain embodiments, a protein hydrolysate and/or amino acid composition is used to replace a casein hydrolysate and/or cysteine. In certain embodiments, a protein hydrolysate and/or amino acid composition produced as described herein improves the viability of a microorganism, e.g., S. thermophilus culture.

Bifidobacterium species are used both as components of starter cultures for fermented milks and as encapsulated freeze-dried material. All bifidobacteria can utilize lactose, which allows them to grow in milk, though the growth is often weak due to low proteolytic activity (Klaver, et al., 1993; Collins and Hall, 1984). However, species such as Bifidobacterium bifidum and Bifidobacterium adolescentis do reportedly produce intracellular and extracellular proteases. Most strains contain a leucine aminopeptidase, while a few have a valine aminopeptidase (Desjardins, et al. 1990). Most bifidobacteria are able to use ammonium salts as their only source of nitrogen (Azaola, et al. 1999), but supplementation of peptides and amino acids are considered a requirement for the economical production of these strains. Specific nitrogen requirements are strain dependent, but the typical nitrogen sources are peptides/amino acids, cysteine, and ammonium salts. Bifidobacteria are reported to have a relatively high demand for growth factors and vitamins, including biotin and calcium pantothenate (Kurmann and Rasic, 1991). Several proteinaceous growth promoters, such as disulfide/sulfhydryl-containing peptides, lactoferrin with bound metals (Fe, Cu, Zn), α-lactalbumin, and β-lactoglobulin are formed in milk (Petschow and Talbott, 1991). In certain embodiments, nutrients such as whole cell biomass and/or lysate and/or protein hydrolysate and/or a proteolytic digest and/or a peptide composition and/or an amino acid composition and/or other nutrient(s) and/or co-factor(s) produced as described herein are combined with milk and/or a culture medium containing lactose, which is then fed to another culture, e.g., a culture of a microorganism that is a different microorganism than the microorganism from which the whole cell biomass and/or lysate and/or protein hydrolysate and/or a proteolytic digest and/or a peptide composition and/or an amino acid composition and/or other nutrient(s) and/or co-factor(s) is derived. In certain embodiments, the whole cell biomass and/or lysate and/or protein hydrolysate and/or a proteolytic digest and/or a peptide composition and/or an amino acid composition and/or other nutrient(s) and/or co-factor(s), which may be combined with other medium components such as lactose, is used in place of milk. In certain embodiments, the culture includes one or more Bifidobacterium strains.

In certain embodiments, a protein hydrolysate and/or peptide composition and/or amino acid composition is a provided to a microorganism culture exhibiting slow growth on a substrate such as milk, e.g., due to low proteolytic activity. In certain embodiments, the culture includes one or more Bifidobacterium strain(s). In certain embodiments, a nutrient, supplement, or medium formulation (e.g., containing a protein hydrolysate and/or peptide composition and/or amino acid composition, produced as described herein), is provided to a culture that includes one or more Bifidobacterium strain(s), and faster growth is observed than the same culture grown on milk alone.

In certain embodiments, a culture containing microorganisms that produce intracellular and/or extracellular proteases are provided nutrients produced as described herein, e.g., including whole cell biomass and/or lysate and/or protein concentrate and/or protein isolate and/or whole proteins, and the protease producing microorganism is able to digest the nutrients to peptides and/or free amino acids that may be used by the microorganism, as well as other microorganisms, for growth and production of biomass and/or bioproduct(s). In certain embodiments, the protease producing microorganism may be, but is not limited to Bifidobacterium bifidum and/or Bifidobacterium adolescentis.

In certain embodiments, a culture is supplemented with peptides and/or amino acids produced as described herein, for the economical production of the culture and/or culture products. In certain embodiments, the culture includes one or more Bifidobacterium strains. In certain embodiments, a nitrogen source is formulated that includes peptides and/or amino acids, including but not limited to, cysteine, produced as described herein. In certain embodiments, the nitrogen source also includes inorganic forms of nitrogen including, but not limited to, ammonium salts. In certain embodiments, the nitrogen source is provided to another culture, e.g., a culture of a microorganism that is a different microorganism than the microorganism from which the nitrogen source is derived, such as, but not limited to, a culture that includes one or more Bifidobacterium strains.

Small peptides are reportedly a better amino acid source than free amino acids for certain Bifidobacterium strains (Proulx, et al., 1994). In certain embodiments, the hydrolysis of proteins produced as described herein is designed to maximize small peptides and minimize free amino acids. In certain embodiments, enzymatic hydrolysis is utilized, to prevent the emergence of high concentrations of free amino acids. In certain embodiments, the peptides with low or no or substantially no free amino acid content is provided to another culture, e.g., a culture of a microorganism that is a different microorganism than the microorganism from which the peptides are derived, such as, but not limited to, a culture that includes one or more Bifidobacterium strains. The selection of proteinase reportedly affects the value of a protein hydrolysate as a growth promoter. Peptides from trypsin-degraded casein are reported to have a better growth promoting effect for Bifidobacterium longum and Bifidobacterium infantis than enzyme digests of Alcalase® or chymotrypsin (Proulx et al., 1994, supra). In certain embodiments, a protein hydrolysate is produced enzymatically from protein produced as described herein (e.g., chemoautotrophically, for example, from a C1 compound, such as CO₂, CO, and/or CH₄, and is provided to another culture, such as, but not limited to, a microorganism culture that includes one or more of B. longum and/or B. infantis.

It has been reported that an Escherichia coli extract used in a complex medium resulted in a significant growth promoting effect for B. longum (Ibrahim and Bezkorovainy, 1994). In certain embodiments, an extract from a chemoautotrophic microorganism, for example, grown on a C1 substrate, such as, but not limited to CO₂, CO, and/or CH₄, is provided to another organism. In certain embodiments, the addition of the extract to the growth medium results in a growth promoting effect in another organism, such as, but not limited to, a Bifidobacterium microorganism, e.g., B. longum.

In certain embodiments, nutrients produced as described herein include growth factors and vitamins such as, but not limited to, biotin and/or calcium pantothenate. In certain embodiments, the growth factors and/or vitamins are provided to another culture, e.g., a culture of a different organism and/or microorganism than the microorganism from which the growth factors and/or vitamins are derived. In certain embodiments, the vitamins include biotin and/or calcium pantothenate and the culture to which these nutrients are provided includes one or more Bifidobacterium strains.

In certain embodiments, nutrients produced as described herein include proteinaceous growth promoters, such as, but not limited to disulfide/sulfhydryl-containing peptides. In certain embodiments, the proteinaceous growth promoters produced as described herein are combined with one or more growth promoters, such as lactoferrin with bound metals (e.g., Fe, Cu, Zn), α-lactalbumin, and/or β-lactoglobulin, from another substrate, such as milk. In certain embodiments, one or more of the aforementioned protein growth promoters are provided to another culture, e.g., a culture of a different organism and/or microorganism than the microorganism from which the proteinaceous growth promoters are derived, such as, but not limited to, a culture that includes one or more Bifidobacterium strains.

D-glucosamine, which is a building block of the peptidoglycan units N-acetylglucosamine and muramic acid, and hence is an essential component of the cell wall, is required by Bifidobacterium sp. (Poupard, et al. (1973), supra). The composition and the strength of the cell wall affects the survival of Bifidobacteria during downstream processing. Milk contains N-acetyl-glucosamine in the form of oligosaccharides, and it is most often used as a glucosamine source (Exterkate and Veerkamp, 1969). In certain embodiments, a whole cell biomass product and/or lysate and/or hydrolysate and/or extract, produced as described herein, includes peptidoglycan and/or peptidoglycan units, such as one or more of: D-glucosamine; N-acetylglucosamine; and/or muramic acid. In certain embodiments, the peptidoglycan and/or peptidoglycan units is provided to another culture, e.g., a culture of a different organism and/or microorganism than the microorganism from which the peptidoglycan and/or peptidoglycan units are derived. In certain embodiments, the peptidoglycan and/or peptidoglycan units are used to replace or supplement peptidoglycan and/or peptidoglycan units, such as N-acetyl-glucosamine, from another source, such as a plant animal source, such as milk. In certain embodiments, the culture requires the peptidoglycan and/or peptidoglycan units for cell wall production and/or growth of the organism, e.g., microorganism. In certain embodiments, provision of the peptidoglycan and/or peptidoglycan units as a medium component and/or supplement improves the composition and cell wall strength of microorganisms in the culture, and/or improves the survival of microorganisms in the culture during downstream processing. In certain embodiments, the culture comprises one or more Bifidobacterium strains.

In addition to complex nutritional requirements, cultivation of Bifidobacteria also requires addressing the extreme sensitivity of these strains to oxygen. This issue is usually overcome by adding substances that can maintain a low redox potential. Cysteine, ascorbic acid, or sodium sulfite are often used for this purpose. In certain embodiments, one or more nutrients and/or biochemicals (e.g., organic compounds) produced as described herein help maintain a low redox potential within a culture medium and/or culture environment to which they are added. In certain embodiments, the redox potential lowering component(s) produced as described herein include, but are not limited to, cysteine and/or ascorbic acid. In certain embodiments, the redox potential lowering component(s)are provided to a culture that includes one or more Bifidobacterium strains.

In certain embodiments, a culture grown on one or more of the nutrients and/or culture media described herein is used as a starter culture for a fermented product, such as a fermented food product, e.g., fermented milks, and/or are further processed into encapsulated freeze-dried materials.

Pediococci are used as components of starter cultures for traditional fermented sausages. Unlike milk, meat is not pasteurized before inoculation with starter cultures and still contains large amounts of indigenous microflora. For the production of Pediococcus sp., glucose or sucrose are used as energy and carbon sources. Although not essential, addition of acetate has been reported to decrease the lag phase and stimulate the growth of the organism. In certain embodiments, nutrients such as whole cell biomass and/or lysate and/or protein hydrolysate and/or proteolytic digest and/or a peptide composition and/or an amino acid composition and/or other nutrient(s) and/or co-factor(s) produced as described herein are combined with glucose and/or sucrose in a culture medium, which is then fed to another culture, e.g., a culture of a different organism and/or microorganism than the microorganism from which the nutrients such as whole cell biomass and/or lysate and/or protein hydrolysate and/or proteolytic digest and/or a peptide composition and/or an amino acid composition and/or other nutrient(s) and/or co-factor(s), produced as described herein, are derived. In certain embodiments, the culture includes one or more Pediococcus strains. In certain embodiments, the culture medium additionally includes acetate. In certain embodiments, the acetate is produced chemoautotrophically, e.g., from a C1 substrate, such as, but not limited to, CO₂, CO, and/or CH₄. In certain embodiments, one or more Pediococcus strains is fermented on a proteinaceous substrate other than meat. In certain embodiments, the proteinaceous substrate includes whole cell biomass and/or lysate and/or protein hydrolysate and/or proteolytic digest and/or a peptide composition and/or an amino acid composition and/or other nutrient(s) or co-factor(s) produced as described herein. In certain such embodiments, a substrate has not been pasteurized before inoculation with starter cultures, such as, but not limited to, one or more Pediococcus strains, and may therefore contain indigenous microflora.

Pediococcus pentosaceus reportedly has the following amino acid requirements: Val, Ala, Met, Pro, Arg, Glu, Cys, Tyr, and His, while other amino acids reportedly have a stimulatory effect. Cys-hydrochloride is reportedly stimulatory but part of its stimulatory effect may be connected to its function as an oxygen scavenger. In certain embodiments, amino acids produced as described herein, including but not limited to, one or more of: Val, Ala, Met, Pro, Arg, Glu, Cys, Tyr, and His, are provided to one or more other strains, e.g., a different organism and/or microorganism than the microorganism from which the amino acids are derived, in the form of free amino acids and/or amino acids bound within peptides and/or proteins. In certain such embodiments, the strain(s) have a requirement for one or more of: Val, Ala, Met, Pro, Arg, Glu, Cys, Tyr, and/or His, for growth. In certain embodiments, the strain(s) include one or more Pediococcus microorganism, such as, but not limited to, Pediococcus pentosaceus.

Pediococcus acidilactici reportedly is able to hydrolyze meat proteins. In certain embodiments, whole cell biomass, lysate, and/or whole proteins produced as described herein is provided to another organism, e.g., a different organism and/or microorganism than the microorganism from which the whole cell biomass, lysate, and/or whole proteins is derived, that is able to hydrolyze them. In certain embodiments, the resultant peptides and/or amino acids resulting from the hydrolysis are then used by the organism itself for nutrition, or are utilized by other organisms for nutrition. In certain embodiments, the resultant peptides and/or amino acids resulting from the hydrolysis are comprised within a final food or feed product. In certain embodiments, the organism or organisms performing the hydrolysis of proteins includes one or more Pediococcus strains, such as, but not limited to Pediococcus acidilactici.

Even when a microorganism strain possesses an adequate proteolytic system for a given protein substrate, growth on specific proteins, such as casein, can be limited due to low levels of certain amino acids, such as His, Leu, Gln, Val, and Met in the example of casein (Kunji, et al., 1995). In certain embodiments, a given proteinaceous substrate is supplemented with one or more of the following as an amino acid source: free amino acids; peptides; protein hydrolysate; protein; lysate; and/or whole cell biomass, produced as described herein, which provide amino acids that are deficient in the proteinaceous substrate. In certain embodiments, provision of the deficient amino acids lifts a limitation on growth for a culture. In some embodiments, the proteinaceous substrate that is supplemented is a milk protein, such as casein. In some embodiments, the amino acids that are supplemented through the provision of the amino acid source, produced as described herein, include but are not limited to, one or more of the following: His; Leu; Gln; Val; and/or Met.

Several Lactobacilli species (L. johnsonii, L. gallinarum, L. gasseri, L. helveticus) are not able to synthesize purines and pyrimidines de novo (Elli, et al., 2000). Milk and milk-derived hydrolysates do not contain purine and pyrimidine precursors. In certain embodiments, purines and/or pyrimidines produced as described herein are provided to one or more microorganism strains that are not able to synthesize purines and/or pyrimidines de novo. In certain embodiments, the strain(s) include Lactobacillus species including but not limited to, one or more of: L. johnsonii, L. gallinarum, L. gasseri, and/or L. helveticus. In certain embodiments, purines and/or pyrimidines produced as described herein are provided to a culture that is grown on milk or milk-derived hydrolysates. In certain embodiments, a composition produced as described herein that includes purines and/or pyrimidines is used to replace another typical source of purines and/or pyrimidines used in culture media, such as, but not limited to, yeast extract.

In certain embodiments, a lysate and/or hydrolysate and/or peptide composition and/or amino acid composition as described herein, supplemented to a nutrient medium, enhances cell proliferation and/or biological production and/or cell density of a cell grown in the nutrient medium. In certain embodiments, the cell is an animal cell. In certain embodiments, a lysate and/or hydrolysate and/or peptide composition and/or amino acid composition as described herein improves viable cell density and/or biomass expansion and/or product yield, in comparison to the same cell grown in an identical medium that does not include the lysate and/or hydrolysate and/or peptide composition and/or amino acid composition. In certain embodiments, a lysate and/or hydrolysate and/or peptide composition and/or amino acid composition as described herein, when included in the nutrient medium, increases cell density. In other embodiments, a lysate and/or hydrolysate and/or peptide composition and/or amino acid composition as described herein improves both cell density and product yield, and in yet other embodiments, suppresses cell growth but enhances yield of one or more bioproduct. In certain embodiments, a lysate and/or hydrolysate and/or peptide composition as described herein contains oligopeptides which act as external molecular signals affecting cell growth and death. In certain embodiments, oligopeptides produced as described herein stimulate cell growth, resulting in higher biomass production, and/or stimulate the production of secreted proteins and/or enhance the viable cell density of the culture. In certain embodiments, oligopeptides produced as described herein act as agents postponing apoptotic death in cell cultures. In certain embodiments, peptides produced as described herein provoke long-lasting shifts in the metabolism of a cell culture and/or alterations in gene expression and/or cell proliferation. In certain embodiments, the shifts and/or alterations last for several days. In certain embodiments, the distribution of cell-cycle phases in a culture medium that includes peptides produced as described herein, is altered. In certain embodiments, peptides produced as described herein regulate proliferation of cultured animal cells and/or signal transduction cascades and/or activate or suppress genes. In certain embodiments, peptides produced as described herein are provided to a cell culture at a concentration greater than or equal to about 1 mM.

In certain embodiments, chromatographic fractions of a lysate and/or hydrolysate and/or peptide composition and/or amino acid composition produce different activities in cell cultures to which they are provided. In certain embodiments, a lysate and/or hydrolysate and/or peptide composition and/or amino acid composition as described herein serves not only as a source of utilizable amino acids, but also as a source of peptides exerting specific effects on cell growth and/or productivity. In certain embodiments, one or more of the following culture parameters is improved through the application of a lysate and/or hydrolysate and/or peptide composition and/or amino acid composition: viable cell density; long-term viability; and/or yield of one or more bioproduct. In certain embodiments, a concentrated mixture of amino acids and/or other nutrients, produced as described herein, increase yield of one or more bioproduct in a culture to which the amino acids and/or other nutrients is provided.

In certain embodiments, the protein hydrolysate composition is added to a culture medium at a concentration of about 0.001% w/v or more, e.g., at about 0.01% w/v or more, at about 0.05% w/v or more, about 0.1% w/v or more, including about 1% w/v or more (as measured by the dry weight of the protein hydrolysate composition). In some embodiments, the protein hydrolysate composition is added to the culture medium in a range of about 0.001% w/v to about 5% w/v, e.g., about 0.01% w/v to about 2% w/v, about 0.05% w/v to about 1% w/v, including about 0.1% w/v to about 1% w/v (as measured by the dry weight of the protein hydrolysate composition). The amount of the protein hydrolysate composition added to the culture medium may vary depending on one or more of a number of considerations, such as cell type, growth, propagation, productivity, differentiation, etc. In certain embodiments, the culture medium is provided to a culture of animal cells.

In certain embodiments, peptides are added to a culture medium at a concentration of about 1 mM, about 2 mM, about 3 mM, about 4 mM, about 5 mM, about 6 mM, about 7 mM, about 1 mM to about 7 mM, about 7 mM to about 10 mM, or greater than about 10 mM. In certain embodiments, oligopeptides produced as described herein are added to the culture medium at a concentration of about 1 mM, about 2 mM, about 3 mM, about 4 mM, about 5 mM, about 6 mM, about 7 mM, about 1 mM to about 7 mM, about 7 mM to about 10 mM, or greater than about 10 mM. In certain embodiments, the culture medium is provided to a culture of animal cells.

In some embodiments, the protein hydrolysate composition added to the culture medium includes a biostimulant polyester. The biostimulant polyester may be a PHA polymer, such as PHB and/or PHV. The biostimulant may be a monomer such as hydroxybutyrate (HB), or an oligomer. The protein hydrolysate composition, when added to the culture medium, provides an effective amount of the biostimulant polyester, e.g., PHA, such as PHB and/or PHV, to the culture medium to promote the growth and/or development of the cultured cells.

In some embodiments, the protein hydrolysate composition added to the culture medium includes a vitamin, such as vitamin B₁, vitamin B₂, and/or vitamin B₁₂, and/or vitamers thereof. The protein hydrolysate composition, when added to the culture medium, provides an effective amount of the vitamin to the culture medium to promote the growth and/or development of the cultured cells. In some embodiments, the protein hydrolysate includes vitamin B₁₂, and/or one or more vitamers thereof (e.g., cyanocobalamin, hydroxocobalamin, adenosylcobalamin and/or methylcobalamin).

In some embodiments, the protein hydrolysate is produced from biomass, which has been produced from a C1 substrate, such as CO₂, CO, and/or CH₄, and which contains vitamin B₁₂ at a concentration relative to the biomass dry weight of about 2 μg/100 g dry biomass, up to about 6.5 μg/100 g dry biomass. In some embodiments, the culture medium produced from C1 substrate contains vitamin B₁₂, e.g., at a concentration relative to the biomass dry weight of about 6.5 μg/100 g dry biomass, up to about 13 μg/100 g dry biomass, or greater than about 13 μg/100 g dry biomass.

In some embodiments, the method includes culturing the cells in a culture medium that includes one or more protein hydrolysates derived from a plant or yeast, in addition to the microorganism, e.g., chemoautotrophic microorganism-derived protein hydrolysate composition as described herein. Suitable plant-based protein hydrolysates include those derived from, without limitation, soy, rice, potato or corn. In some embodiments, the method includes culturing the cells in a culture medium that includes a plant-based protein hydrolysate and a microorganism, e.g., chemoautotrophic microorganism-derived protein hydrolysate composition as described herein. In some embodiments, the method includes culturing the cells in a culture medium that includes a yeast-based protein hydrolysate and a microorganism, e.g., chemoautotrophic microorganism-derived protein hydrolysate composition as described herein. In some embodiments, the method includes culturing the cells in a culture medium that includes a yeast-based protein hydrolysate, a plant-based protein hydrolysate and a microorganism, e.g., chemoautotrophic microorganism-derived protein hydrolysate composition as described herein. Suitable plant- and/or yeast-based protein hydrolysates are described in, e.g., U.S. Pat. No. 8,093,045 and PCT publication number WO1999057246, which are hereby incorporated by reference in their entireties.

The plant- or yeast-based protein hydrolysate may be added to the culture medium in any suitable amount. In certain embodiments, the plant- or yeast-based protein hydrolysate is added to the culture medium at a concentration of about 0.001% w/v or more, e.g., at about 0.01% w/v or more, at about 0.05% w/v or more, about 0.1% w/v or more, including about 1% w/v or more. In some embodiments, the plant- or yeast-based protein hydrolysate is added to the culture medium in a range of about 0.001% w/v to about 5% w/v, e.g., about 0.01% w/v to about 2% w/v, about 0.05% w/v to about 1% w/v, including about 0.1% w/v to about 1% w/v.

The cells may be cultured in the culture medium with the microorganism, e.g., chemoautotrophic microorganism-derived protein hydrolysate composition as described herein for any suitable amount of time. In some embodiments, the cells are cultured continuously throughout growth and propagation in the presence of the protein hydrolysate in the culture medium. In some embodiments, the cells are cultured in the culture medium containing the chemoautotrophic microorganism-derived protein hydrolysate for about one hour or more, e.g., about 5 hours or more, about 12 hours or more, about 24 hours or more, about 5 days or more, about 2 weeks or more, about 6 weeks or more, about 3 months or more, about 6 months or more, including about one year or more. In some embodiments, the cells are cultured in the culture medium containing the chemoautotrophic microorganism-derived protein hydrolysate for a period of about 1 hour to about 3 years, e.g., about 5 hours to about 1 year, about 12 hours to about 6 months, about 24 hours to about 3 months, including about 5 days to about 6 weeks.

In some embodiments, the microorganism, e.g., chemoautotrophic microorganism-derived protein hydrolysate composition is present temporarily in the culture medium during culturing of the cells. When the cells are grown in the absence of the microorganism-derived protein hydrolysate in the culture medium, other media supplements, such as a plant- or yeast-derived protein hydrolysate, may be present in the culture medium. Any suitable cells may be cultured using the present method. The cells may be derived from a mammal, bird, fish, insect, or other animal source. The cells may be stem cells. The cells may be fungal, plant, eukaryotic, or prokaryotic. The cells may be a probiotic. Cultured cells may be primary cells, immortalized cell lines, hybridomas, established cell lines, stem-cell derived cells, or genetically engineered cells, such as recombinant cells expressing a heterologous polypeptide or protein. The cells may be individual cells, tissues, organs. Suitable non-mammalian animal cells include insect cells, avian cells (including chicken cells), and piscine cells. Suitable cells include mammalian cells of human or non-human origin. Suitable mammalian cells include, without limitation, bovine, porcine, ovine, leporine, or equine cells. The cultured cells can be monkey kidney cells, bovine kidney cells, dog kidney cells, pig kidney cells, rabbit kidney cells, mouse kidney cells, rat kidney cells, sheep kidney cells, hamster kidney cells, Chinese hamster ovarian cells or an animal cell derived from any tissue. Suitable mammalian cells include, without limitation, CHO cells, COS cells, VERO cells, HeLa cells, 293 cells, HEK-293 cells, HEK cells, PER.C6 cells, K562 cells, MOLT-4 cells, M1 cells, NS0 cells, NS-1 cells, COS-7 cells, MDBK cells, MDCK cells, MRC-5 cells, WI-38 cells, WEHI cells, SP2/0 cells, BHK cells, stem cells, and derivatives thereof. Suitable non-mammalian cells include, without limitation, AGE1.CR cells, EB66 cells, Sf9 cells, stem cells, and derivatives thereof. In certain embodiments, the culture cells grown on medium components produced as described herein have been transfected with exogenous nucleic acid.

In some embodiments, the method includes culturing myocytes in a culture medium containing a microorganism, e.g., chemoautotrophic microorganism, derived lysate and/or protein hydrolysate and/or protein hydrolysate and/or peptide composition and/or amino acid composition as described herein. In some embodiments, the method includes culturing cells in the culture medium to generate and maintain myocytes. Suitable cells for generating myocytes include, without limitation, embryonic stem cells, satellite cells and myoblasts. In some embodiments, the cells include adipocytes. In some embodiments, the cells include fibroblasts. In some embodiments, any two or more of myocytes, adipocytes and fibroblasts are cultured together in a culture medium containing a microorganism, e.g., chemoautotrophic microorganism-derived protein hydrolysate composition as described herein.

The cells may be cultured using any suitable method. The cells may be grown in suspension, roller bottles, flasks and the like. Large scale approaches, such as bioreactors, including adherent cells growing attached to microcarriers in stirred fermenters, also are included. In some embodiments, the cells are grown in suspension. If the cells are grown on microcarriers, the microcarrier can be selected from the group of microcarriers based on dextran, collagen, plastic, gelatin and cellulose and others. In certain embodiments, the microcarriers may comprise PHA (e.g., PHB and/or PHV) produced from microorganisms as described herein. In certain embodiments, microcarrier support systems may be used in pseudo-suspension cultures within stirred tank bioreactors.

In some embodiments, the method includes culturing the cells on a three-dimensional support or scaffold. The scaffold may provide a microenvironment suitable to support proliferation, maintenance, development and/or differentiation of the cells in the presence of microorganism, e.g., chemoautotrophic microorganism-derived protein hydrolysate composition in the culture medium. In some embodiments, the three-dimensional support or scaffold is porous. In certain embodiments, the three-dimensional support or scaffold includes PHA (e.g., PHB and/or PHV) produced from microorganisms as described herein.

The three-dimensional support or scaffold may be made of any material suitable for the cultured cells to grow thereon. In some embodiments, the scaffold is biodegradable. In some embodiments, the scaffold is made of a biodegradable material, such as a biodegradable polyester or hydrogel. In some embodiments, the scaffold is made of a PHA polymer, such as, but not limited to PHB. In some embodiments, the biodegradable polyester, such as PHA or PHB, is produced by a microorganism, e.g., chemoautotrophic microorganism. In certain embodiments, the scaffold is consumable, e.g., suitable for human consumption. An edible support or scaffold may be made of, without limitation, gellan gum, alginate, pectin, or cellulose. In certain embodiments the scaffold is 3-D printed.

The cells may be cultured at an appropriate temperature and pH. Mammalian cells are typically cultivated in a cell incubator at about 37° C., with the culture medium having an optimal pH in the range of about 6.8 to 7.6, including between 7.0 and 7.3. In some embodiments, cells in batch culture might have a complete medium change about every 2 to 3 days, or more or less frequently, if required. Cells in perfusion culture (e.g., in bioreactor or fermenter) might have a fresh media change on a continuously recirculating basis.

In certain embodiments, the method includes culturing animal cells to produce a product suitable for human consumption, such as a meat product. Thus, provided herein are methods of culturing meat using a culture medium containing a microorganism, e.g., chemoautotrophic microorganism, derived lysate and/or protein hydrolysate and/or peptide composition and/or amino acid composition as described herein to culture myocytes, with or without other cells (such as adipocytes or fibroblasts). Where the culturing medium is a serum-free or animal component-free medium, the present method provides a humane process for producing meat products. In certain embodiments, an animal derived serum is used in the medium; however, some fraction of the amino acids or protein hydrolysate are derived from a microorganism as described herein.

In some embodiments, the microorganism-derived protein hydrolysate composition includes components that improve the flavor of the cultured food product, e.g., cultured meat. In certain embodiments, the protein hydrolysate composition stimulates the development of flavor-enhancing elements in the cultured food product, e.g., cultured meat. As used herein, enhancing the flavor of a food product includes rendering the product more palatable, or imparting one or more flavor components that are found in the naturally-produced counterpart of the cultured product.

In some embodiments, the method includes culturing myocytes in a serum-free culture medium that contains a microorganism, e.g., chemoautotrophic microorganism, derived whole cell biomass and/or lysate and/or extract and/or protein hydrolysate and/or peptide composition and/or amino acid composition as described herein. In some embodiments, the method includes culturing myocyte precursor cells in a serum-free culture medium that contains a microorganism, e.g., chemoautotrophic microorganism, derived whole cell biomass and/or extract and/or lysate and/or protein hydrolysate and/or peptide composition and/or amino acid composition as described herein. In some embodiments, the method includes culturing myocytes in a culture medium that includes animal-derived serum, but which also contains a microorganism, e.g., chemoautotrophic microorganism, derived protein whole cell biomass and/or extract and/or lysate and/or protein hydrolysate and/or peptide composition and/or amino acid composition as described herein. In some embodiments, the method includes culturing myocyte precursor cells in a culture medium that includes animal-derived serum, but which also contains a microorganism, e.g., chemoautotrophic microorganism, derived whole cell biomass and/or extract and/or lysate and/or protein hydrolysate and/or peptide composition and/or amino acid composition as described herein. The myocyte precursor cells may be cultured under conditions sufficient to promote propagation of the precursor cells in the culture medium. In some embodiments, the myocyte precursor cells may be cultured under conditions sufficient to induce differentiation of the precursor cells into myocytes. The precursor cells may be any suitable cells that can be induced to give rise to myocytes. Suitable precursor cells include, without limitation, satellite cells, embryonic stem cells, and myoblasts. Thus, the present method includes adding an effective amount of the chemoautotrophic microorganism-derived protein hydrolysate to the culture medium to promote proliferation, maintenance, development and/or differentiation of myocytes.

In certain embodiments, the myocytes are cultured with one or more other cell types. Suitable cells for co-culturing with the myocytes include, without limitation, adipocytes and fibroblasts, or precursors thereof. Thus, the present method may include culturing cells in a serum-free culture medium, or culture medium containing animal-derived serum, that contains a microorganism, e.g., chemoautotrophic microorganism, derived whole cell biomass and/or lysate and/or extract and/or protein hydrolysate and/or peptide composition and/or amino acid composition as described herein to produce a collection of muscle cells, fat cells, and connective tissue. The cells may be cultured under conditions sufficient to induce myogenesis, or muscle fiber formation. In some embodiments, the method includes adding an effective amount of the microorganism, e.g., chemoautotrophic microorganism, derived whole cell biomass and/or lysate and/or extract and/or protein hydrolysate and/or peptide composition and/or amino acid composition to the culture medium to promote proliferation, maintenance, development and/or differentiation of adipocytes and/or fibroblasts. In some embodiments, the method includes adding an effective amount of the microorganism, e.g., chemoautotrophic microorganism, derived whole cell biomass and/or lysate and/or extract and/or protein hydrolysate and/or peptide composition and/or amino acid composition to the culture medium to induce, sustain and/or promote myogenesis.

In some embodiments, the myocytes (with or without adipocytes and/or fibroblasts) are cultured on a three-dimensional support or scaffold. The scaffold in some cases may be porous. The scaffold may be made of any suitable material for supporting growth of the cells and/or myogenesis. The scaffold may be a biodegradable and/or consumable material. In some embodiments, the scaffold includes a biodegradable polyester, or a biodegradable hydrogel. In certain embodiments, the scaffold includes a PHA polymer, such as PHB and/or PHV. In some embodiments, the scaffold is made of material derived from a microorganism, e.g., chemoautotrophic microorganism. In some embodiments, the scaffold includes a biodegradable polyester, such as PHA, e.g., PHB and/or PHV, produced sustainably by growth, e.g., chemoautotrophic growth, of the microorganism.

In some embodiments, an effective amount of the microorganism, e.g., chemoautotrophic microorganism, derived whole cell biomass and/or lysate and/or extract and/or protein hydrolysate and/or peptide composition and/or amino acid composition as described herein is added to a culture medium to provide a source of vitamins to growing cells. The whole cell biomass and/or lysate and/or extract and/or protein hydrolysate and/or peptide composition and/or amino acid composition may be a source of vitamins, such as, but not limited to, vitamin B₁, vitamin B₂, and/or vitamin B₁₂, e.g., for myocytes and/or other cells. The whole cell biomass and/or lysate and/or extract and/or protein hydrolysate and/or peptide composition and/or amino acid composition may be a source of one or more vitamins, including, but not limited to: vitamin A; beta-carotene, lutein, zeaxanthin, thiamine (B₁), riboflavin (B₂), niacin (B₃), pantothenic acid (B₅), vitamin B₆, folate (B₉), vitamin B₁₂, choline, vitamin C, vitamin D, vitamin E, vitamin K, e.g., for myocytes and/or other cells. The whole cell biomass and/or lysate and/or extract and/or protein hydrolysate and/or peptide composition and/or amino acid composition may be a source of one or more minerals, including but not limited to: calcium, iron, magnesium, manganese, phosphorus, potassium, sodium, zinc, e.g., for myocytes and/or other cells.

In certain embodiments, a whole cell biomass and/or lysate and/or extract and/or protein hydrolysate produced as described herein serves as a source of one or more of: amino acids; oligopeptides; lipids; and/or iron, for a culture of cells that are different cells than the microorganism cells from which the amino acids, oligopeptides, lipids, and/or iron are derived.

In some embodiments, a food product (for example, but not limited to, a meat product or meat-like product) produced by a method as described herein may be a nutritional source of vitamins, such as vitamin B₁, vitamin B₂, and/or vitamin B₁₂. In certain embodiments, the food product contains vitamins, such as vitamin B₁, vitamin B₂, and/or vitamin B₁₂. In certain said embodiments, the vitamin B₁, vitamin B₂, and/or vitamin B₁₂ are provided through the whole cell biomass and/or lysate and/or protein hydrolysate and/or peptide composition and/or amino acid composition and/or other extracts, produced as described herein, added to the culture medium for the cultured cells that are used to produce at least a portion of the food product. In certain embodiments, the food product contains one or more vitamins, including but not limited to: vitamin A; beta-carotene, lutein, zeaxanthin, thiamine (B₁), riboflavin (B₂), niacin (B₃), pantothenic acid (B₅), vitamin B₆, folate (B₉), vitamin B₁₂, choline, vitamin C, vitamin D, vitamin E, and/or vitamin K, some or all of which may be ultimately sourced from the microorganisms from which the whole cell biomass and/or lysate and/or protein hydrolysate and/or peptide composition and/or amino acid composition and/or extract is derived. In certain embodiments, the vitamins are provided through the whole cell biomass and/or lysate and/or extract and/or protein hydrolysate and/or peptide composition and/or amino acid composition, added to the culture medium for the cultured cells that are used to produce at least a portion of the food product.

In some embodiments, a food product (e.g., meat or meat-like product) produced by a method as described herein may be a nutritional source of minerals, including but not limited to, one or more of: calcium, iron, magnesium, manganese, phosphorus, potassium, sodium, and/or zinc. In certain embodiments, the minerals are provided through whole cell biomass and/or lysate and/or protein hydrolysate and/or peptide composition and/or amino acid composition and/or other extracts, produced as described herein, added to the culture medium for the cultured cells that are used to produce the food product. In certain embodiments, the iron content of the whole cell biomass and/or lysate and/or extract and/or protein hydrolysate and/or peptide composition and/or amino acid composition includes iron in the heme form.

In certain embodiments, a formulation is provided that includes whole cell biomass and/or lysate and/or extract and/or protein hydrolysates and/or a peptide composition and/or an amino acid composition and/or other nutrients or co-factors, produced as described herein, in combination with other ingredients, including but not limited to, biomass and/or whole cells and/or protein concentrate and/or protein isolate and/or protein hydrolysate and/or extract from one or more of the following sources: meat; dairy; egg; soy; wheat; rice; pea; other plant proteins; yeast; probiotics; LAB; and/or other GRAS micro- or macro-organisms. In certain embodiments, a yogurt mix is supplemented with one or more of: whole cell biomass and/or lysates and/or protein hydrolysates and/or peptide compositions and/or amino acid compositions and/or other nutrients or co-factors produced as described herein. In certain embodiments, the formulation does not include proteins or other biomass components from meat and/or soy sources. In certain embodiments, the formulation does not include proteins, protein hydrolysates and/or other biomass components from dairy and/or non-soy vegetable origins. In some cases, the formulation does not include genetically modified microorganisms (i.e., is GMO-free). In some cases, the formulation is meat and/or dairy free. In certain embodiments, the formulation is utilized in a food, feed, or beverage product. In certain embodiments, the food, feed, or beverage product is vegetarian or vegan. In certain embodiments, formulation is considered GRAS for human consumption.

In certain embodiments, a culture of cells grown on a whole cell biomass and/or lysate and/or extract and/or protein hydrolysate and/or peptide composition and/or amino acid composition as described herein secretes a protein product. In certain embodiments, protein purification processes are utilized to recover and/or purify the protein product. Any suitable protein purification method, as are well known in the art, may be used to recover and/or purify the protein product.

In certain embodiments, after products have been produced using a whole cell biomass and/or lysate and/or extract and/or protein hydrolysate and/or peptide composition and/or amino acid composition as component(s) of a culture medium, as described herein, removal of whole cell biomass and/or lysate and/or extract and/or protein hydrolysate and/or peptide composition and/or amino acid composition material from the culture medium or fermentation broth and/or products are performed. In certain cases, such removed nutrients are recycled back to one or more upstream steps for more complete utilization, and/or are used as a co-product.

The following examples are intended to illustrate, but not limit, the invention.

### EXAMPLES

### Example 1: Protein Hydrolysate Produced from a Cupriavidus necator Culture

A Cupriavidus necator strain was cultivated chemoautotrophically in a mineral salts growth medium with CO₂ as carbon source and H₂ as electron donor. After growth, whole cell biomass was isolated from the growth medium by centrifugation and dried by lyophilization. The dried biomass was processed as follows.

Defatting the whole cell biomass: The whole cell biomass was defatted (lipids extracted out) with ammonium hydroxide and methanol (1:1:0.4, WCB: NH₄OH: MeOH) by stirring the mixture for an hour in a fume hood in a tightly capped container. The mixture was vacuum filtered with Whatman 4 filter paper. The filtrate contained the extracted lipids. The retentate on the filter was the defatted biomass, which was dried at 40° C. in an incubator overnight.

Protein hydrolysis with NH₄OH and neutralization with CO₂: Solid loading of 2% of defatted dried mass into the hydrolysis step was prepared by rehydrated with the required amount of deionized (DI) water. The slurry was mixed well with a Turret Stick at 15,000 rpm for 1 min. The pH of the reaction mix was increased to 10.85 using NH₄OH 28%-30% (pre-made) solution in a fume hood. The mixture was transferred to a pressure tube (size: 120 mL) with 50 mL working volume. The mixture was then autoclaved at 110° C., 10 min, slow exhaust. The pH of the solution post autoclave was 10.82. The pH was then decreased to pH 9 by bubbling CO₂ through a cannula/18 G needle inserted into the solution for 10-20 min. Enzyme digestion then was done at pH 9 with Bacterial Alkaline Protease at 55° C., 110 rpm overnight. The supernatant containing soluble hydrolyzed proteins was separated from the PHB rich crude pellet by centrifugation at 20000×g, 20 min, 5 C. The supernatant protein hydrolysate was then freeze dried. The ash content measured for this protein hydrolysate (PH) was 5%. The ash content was measured by placing a minimum of 300 mg of protein hydrolysate powder in a tared crucible, and running an ash cycle in a muffle furnace. It was also independently measured by external lab analysis (SGS, North America) using AOAC method 942.05. The total amino acid content of the PH and the amino acid profile was also determined by SGS, North America using the AOAC method AOAC 994.12. Results are shown in Table 1.

TABLE 1
NH₄OH/CO₂ Low Ash PH

| % Total Amino Acids | 84.96 |
|---|---|
| Sample Form | Dry powder |
| % Ash | 4.99 |
| Amino Acid | % of total dry weight (w/w) |
| Cysteine | 0.13 |
| Methionine | 1.62 |
| Tryptophan | 0.95 |
| Alanine | 7.7 |
| Arginine | 6.19 |
| Aspartic Acid | 6.1 |
| Glutamic Acid | 10.34 |
| Glycine | 5.11 |
| Histidine | 1.59 |
| Isoleucine | 3.04 |
| Leucine | 7.36 |
| Lysine | 6.28 |
| Phenylalanine | 4.95 |
| Proline | 3.77 |
| Serine | 6.1 |
| Threonine | 3.8 |
| Tyrosine | 4.41 |
| Valine | 5.52 |

### Example 2: Protein Hydrolysate Produced from a Cupriavidus necator Culture

A Cupriavidus necator PHB negative mutant strain (DSM 541) was cultivated chemoautotrophically in an inorganic mineral growth medium on CO₂ as a carbon source and H₂ as electron donor. After growth, the whole cell biomass was isolated from the growth medium by centrifugation and dried by lyophilization. The dried biomass was processed as follows.

Defatting the whole cell biomass: Dry whole cell biomass was defatted via treatment with ammonium hydroxide and ethanol (1:4:0.5 w/v). The biomass and solvent slurry were stirred for 30 minutes in a tightly capped glass bottle before being vacuum filtered through Whatman 4 filter paper in a fume hood. The filtrate contained the lipid fraction. The defatted retentate recovered from the filter was air dried overnight before being dried at 40° C. in an incubator for 4-6 hours.

Protein hydrolysis on defatted biomass with Ca(OH)₂ and neutralization with H₃PO₄: Dried defatted biomass was resuspended in DI water to a final concentration of 2%. The biomass solution was mixed with IKA Ultra-Turax at 15000 rpm until fully and homogeneously resuspended. The biomass solution was brought to a pH of 11 by addition of Ca(OH)₂. The solution was transferred to a glass media bottle and autoclaved at 110° C. for ten minutes and subsequently cooled to room temperature. The solution was neutralized to pH 9 using H₃PO₄. Bacterial alkaline protease (Sigma P8038) was added to the solution at a concentration of 2.6 active units/g biomass. The biomass solution was placed in a 55° C. shaking water bath overnight. After 16-24 hour digestion, the enzyme was inactivated by incubating the slurry in a 95° C. water bath for ten minutes. The biomass slurry was then cooled to room temperature and the protein hydrolysate (supernatant fraction) was separated by centrifugation at 26000×g for 30 minutes at 7° C. The resulting protein hydrolysate solution was frozen in a −80° C. freezer before being lyophilized. The moisture, ash, and N content of the dried powder was determined and the protein profile was analyzed via SDS-PAGE analysis. There were no proteins above 2000 dalton present in the protein hydrolysate and the resulting ash content was 10.5%. The total amino acid content of the PH and the amino acid profile was also determined by SGS, North America using the AOAC method AOAC 994.12. The mineral profile was determined using AOAC method AOAC 968.08. Results are shown in Table 2.

TABLE 2

| | Result |
|---|---|
| Amino Acid | % total dry weight |
| Analysis | (w/w) or PPM) |
| Total Amino Acids | 69.92% |
| Cysteine | 0.25% |
| Methionine | 1.57% |
| Tryptophan | 0.65% |
| Alanine | 5.99% |
| Arginine | 4.96% |
| Aspartic Acid | 7.35% |
| Glutamic Acid | 8.88% |
| Glycine | 4.01% |
| Histidine | 1.41% |
| Isoleucine | 2.8% |
| Leucine | 5.86% |
| Lysine | 5.33% |
| Phenylalanine | 3.23% |
| Proline | 4.52% |
| Serine | 2.73% |
| Threonine | 3.55% |
| Tyrosine | 2.28% |
| Valine | 4.55% |
| Calcium | 1.43% |
| Copper | 3.36 PPM |
| Iron | 28.62 PPM |
| Potassium | 1.29% |
| Magnesium | 0.13% |
| Manganese | N.D. |
| Sodium | 1.18% |
| Phosphorus | 1.17% |
| Zinc | N.D. |

### Example 3: Protein Hydrolysate Design

For a variety of different digestion processes (acid versus alkali versus enzyme hydrolysis versus autolysis), choice of enzymes (purified or blends of exogenous animal/plant/microbial enzymes and/or endogenous enzymes) that have different enzyme specificities, and process parameters (pH, temperature and incubation/processing time), analysis of the resultant protein hydrolysate produced from chemoautotrophic biomass is performed. The release of peptides and size of peptides, represented as a peptide profile, is performed. The average molecular weight (MW) of the peptides (Da) is determined. The ratio of amino nitrogen to total nitrogen (AN/TN ratio) and degree of hydrolysis (DH %) is measured, as well as the levels of free amino acids. The effect of various hydrolyzing agents is compared on the basis of DH % and the peptide and amino acid distribution profiles. A comparison of protein hydrolysates produced as described herein is also performed against common and/or commercial sources of protein hydrolysates, including but not limited to, dairy, meat, and/or soy protein hydrolysates and/or yeast extracts, produced via a variety of hydrolysis methods, including enzymatic, acidic, alkali, and/or autolysis. This comparison is performed on the basis of total amino acid profiles, AN/TN ratios, as well as all of the other parameters and characteristics outlined above.

The comparison is also performed on the basis of a culture's growth performance when provided a given protein hydrolysate. This test culture can include one or more LAB. The LAB can include a L. helveticus strain. The protein hydrolysates to compare against can include a casitone-pancreatic digest of casein (casitone)—a peptone made by the pancreatic digestion of casein. Measures of culture growth performance includes the cell numbers measured over time and specific growth rate. One negative control for the test can be the base culture medium without any protein hydrolysate supplementation. Another negative control can be the culture medium without any protein hydrolysate supplementation or any amino acid media component. Another experimental test and comparison is between different protein hydrolysates, including one prepared as described herein, in a media where the protein hydrolysates constitute the sole source of amino acids (i.e., no other amino acid components included in the media). In addition to comparisons against other protein hydrolysates, comparisons are also made against media without protein hydrolysate supplementation, but with free amino acid compositions such as casamino acids, or with whole proteins, such as casein.

### Example 4: Testing Whole Cell Biomass, Lysate and Hydrolysate Effect on Beneficial Fungus Trichoderma atroviride

Trichoderma atroviride is a saprophytic fungus living in the rhizosphere and the soil in a wide range of temperatures and soil pH conditions. It is beneficial to many different crops due to its ability to counteract phytopathogenic fungi including Rhizoctonia solani and Botrytis cinerea, which cause disease in hundreds of plant crops, including tomatoes, beans, cucumber, strawberries, cotton and grapes. It also increases micronutrient uptake by plants and is associated with root growth stimulation. Trichoderma atroviride is largely used as a microbial inoculant in agricultural cropping systems.

The aim of this in vitro experiment was to evaluate the stimulation activity of seven different lysates and protein hydrolysates as well as whole cell biomass (WCB) produced as described herein from Cupriavidus necator biomass that was grown autotrophically on CO₂ as the carbon source using H2 as an electron donor. These seven biomass products were compared against two commercial biostimulants; one comprising a plant-derived PH, and the other an animal derived PH.

The different WCB, lysate, and PH samples were evaluated for their ability to promote growth of Trichoderma atroviride AT10 in sterile substrate. Pure culture of Trichoderma atroviride AT10 was grown on potato dextrose agar (PDA) culture medium as control, and on PDA medium supplemented with 3 ml/L of the nine WCB, lysate, and PH samples for 24 hours at 25° C. in the darkness.

PDA medium enriched with the lysate and PH products were prepared by dissolving the required amount of each organic product in deionized water. The resulting solutions were filtered, by using a 0.25 μm filter, into sterile bottles containing PDA at 45° C. The substrates were gently stirred and then were poured into separate 9-cm petri dishes. When substrates in the plates had cooled down and solidified, 5 mm-mycelia disks of Trichoderma atroviride AT10, taken from the margin of the pure culture after 4 days growth, were placed in the center of each petri plate, and incubated at 25° C. Radial growth of the mycelium was measured after 24 hours of incubation. There were twelve petri dishes per treatment. All data were statistically analyzed with SPSS v. 21 (IBM Corp., Armonk, N.Y., USA). Duncan's multiple range test was performed at P=0.05 on each of the significant variables measured. In Table 3, the mean values are followed by standard errors. Different letters within each column indicate significant differences according to Duncan's multiple range test P=0.05.

TABLE 3

| | Radial growth of mycelium | |
|---|---|---|
| Treatment | (mm) | |
| Control | 9.08 | ±0.19 cd |
| C. necator derived PH | 11.00 | ±0.44 a |
| Animal derived PH | 8.00 | ±0.44 e |
| Plant derived PH | 10.33 | ±0.56 ab |

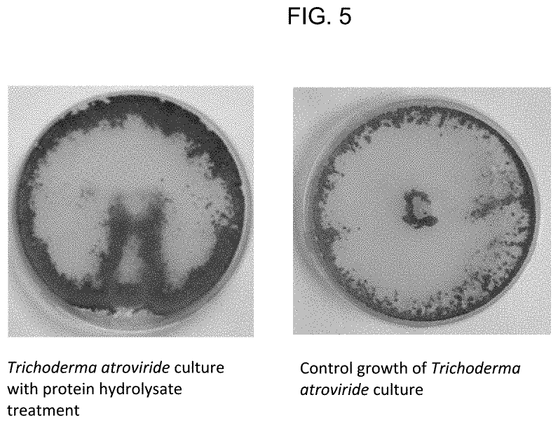

It was found that PDA substrate enriched with organic products could significantly affect radial growth of Trichoderma mycelium after 24 hours of incubation. The highest Trichoderma growth was recorded for a PH produced from C. necator through base treatment followed by protease treatment. The commercial biostimulant based on plant derived PH has lower average growth, but was not significantly different. The PH produced from C. necator through base treatment followed by protease treatment produced significantly greater growth than the control and the commercial biostimulant based on animal derived PH. Visually, a substantially denser spore formation is observed after the treatment with C. necator PH compared to the control (FIG. 5) with spores being a means of Trichoderma reproduction and propagation.

The results of this preliminary trial demonstrated that PH produced from C. necator through base treatment followed by protease treatment has the potential to enhance in vitro Trichoderma growth. PH from C. necator may be applied to the rootzone of a crop for stimulating root growth and nutrient uptake and to boost the soil population of naturally-occurring or artificially-inoculated Trichoderma species.

### Example 5: Testing Addition of Proteinaceous Cupriavidus necator Biomass to Tempeh Fermentation Substrate

The goal was to test whether high protein biomass from Cupriavidus necator grown on CO₂ could be used to supplement a plant substrate used to produce tempeh.

A commercial starter culture of Rhizopus oryzae was acquired.

Chickpeas were soaked overnight in sterilized water (boiled), and then cooked in an instant pot under normal pressure mode for one minute. The cooked chickpeas were just tender and soft enough to bite through. Then the chickpeas were drained quickly while still hot. The chickpeas were then dried by spreading on a paper towel in a thin layer. The beans were dehulled.

The chickpeas were then split into two flat wide mouth plastic containers. Container #1 served as a control where only chickpeas were added, In container #2, chickpeas were added along with dried whole cell Cupriavidus necator biomass, which was proportioned at 10% of the starting chickpeas weight.

Vinegar was added gradually to both the containers, and mixed well to make the starting pH 3.5.

1 teaspoon of the Rhizopus oryzae starter culture per pound dry weight of cooked beans was sprinkled over the substrate. Then the chickpeas+culture were mixed well with a spatula so that culture stuck evenly to all the substrate.

Both the wide mouth plastic containers (i.e., the chickpea control and the chickpea+C. necator experiment) had a lid with holes punched 0.3-0.5″ apart and the lid was loosely kept on top. The containers were transferred to the incubator at 30.5° C. and incubated for 48-72 hrs.

R. oryzae white mycelial growth started after 20-24 hrs. of incubation, after this the fermentation substrate was transferred from the incubator to room temperature. Complete R. oryzae mycelial growth was observed after 48-72 hrs.

It was observed that R. oryzae grew equally well, with even white mycelial growth, on Chickpeas with or without C. necator biomass added. Thus, tempeh can be supplemented with protein and other nutrients (e.g., B vitamins) provided by C. necator.

Further analysis is performed to investigate any compositional or nutritional differences between the supplemented and unsupplemented tempeh.

Similar experiments can be performed, testing out other edible fungal strains used for producing tempeh or other fermented products like miso or soy sauce such as Aspergillus oryzae (NRRL 3485), Rhizopus oligosporus (NRRL 2710), and Rhizopus oryzae (NRRL 3613). Additional fermentation substrates can also be tested such as white rice, barley, mung bean, okara flour, and soybeans.

### Example 6: Test Growth of Lactic Acid Bacteria on Protein Hydrolysates and Protein Isolate

GRAS lactic acid bacteria (LAB) are tested on nutrients derived from Cupriavidus necator proteinaceous biomass produced from CO₂. The protein-derived nutrients to be tested include: an alkaline protein hydrolysate (PH) produced by a base treatment with NH₄OH followed by neutralization with CO₂ and enzymatic hydrolysis using bacterial alkaline protease (BAP); an acid protein hydrolysate produced by an acid treatment with H₃PO₄ followed by neutralization with Ca(OH)₂ and then enzymatic hydrolysis with BAP; and a protein isolate (PI) formed by sonication of the biomass followed by centrifugation, discard of the pellet, and recovery, and drying of the protein-rich supernatant.

These and other protein derived products are tested on LAB strains including but not limited to S. thermophilus, L. delbrueckii subsp. Bulgaricus, L. acidophilus, and Bifidobacterium lactis. LAB strains will be tested individually as well as in co-cultures, mixed cultures, and consortiums. The growth medium used in controls is regular cow's milk in one type of control, and Elliker broth (Sigma #17123) in another type of control. The composition of Elliker broth is: 0.5 g/L ascorbic acid; 20 g/L casein enzymic hydrolysate; 5 g/L dextrose; 2.5 g/L gelatine; 5 g/L lactose; 5 g/L saccharose; 1.5 g/L sodium acetate; 4 g/L sodium chloride; 5 g/L yeast extract.

The control case of yogurt from milk is produced as follows: 1-2 quarts of pasteurized whole milk will be heated to 180° F., and then cooled down to 115° F. The milk is poured into a glass container and one packet of commercial yogurt starter culture comprising S. thermophilus, L. delbrueckii subsp. Bulgaricus, L. acidophilus, and Bifidobacterium lactis will be added and mixed thoroughly. The milk culture is then covered and incubated at 105-112° F. for approximately 8 hrs in the Instant Pot on yogurt mode. The culture is checked by tilting the jar gently. When the yogurt moves away from the side of the jar in one mass instead of running up the side, it is finished culturing. Once the yogurt has set, it is covered and allowed to cool for 2 hrs at room temperature and then refrigerated.

The control case of LAB growth on Elliker broth is as follows: Suspend 48.5 grams of Elliker broth power in one liter distilled water. Mix and boil to dissolve the medium completely and then sterilize by autoclaving at 121° C. for 15 minutes. The medium solution is light to medium amber clear and the pH between 6.6 to 7. Cool down the solution to 115° F. and inoculate one packet of culture of commercial yogurt starter culture comprising S. thermophilus, L. delbrueckii subsp. Bulgaricus, L. acidophilus, and Bifidobacterium lactis per 1-2 quarts of media. The medium is then covered and cultured at 105-112° F. for approximately 24 to 48 hrs. The optical density at 600 nm (OD₆₀₀) of the LAB culture in Elliker broth will be monitored over time.

One experiment that is tested against the milk control involves mixing C. necator derived PH or PI into milk in a 1:1 ratio of C. necator nitrogen (N) to milk nitrogen, and then carrying out the same yogurt fermentation steps as described above.

Another experiment that is tested against the Elliker broth control involves replacing the casein enzymatic hydrolysate in the Elliker broth with an amount of test substrate (PH/PI) giving the same N content in the broth as the 20 g/L casein enzymatic hydrolysate. The rest of the media composition is kept the same. LAB growth performance on the experimental broth is compared against the Elliker broth control in terms of growth rate and final titer. Similar experiments involve replacing both the casein enzymatic hydrolysate and the gelatin in the Elliker broth with experimental PH and/or PI on an equal nitrogen basis; and replacing casein enzymatic hydrolysate, gelatin, and yeast extract in the Elliker broth, with the casein enzymatic hydrolysate and gelatin replaced on an equal nitrogen basis, and the yeast extract replaced on an equivalent B vitamin basis, or nucleotide basis.

### Example 7: Testing Protein Hydrolysate Effect on Cell Viability and other Performance Metrics

The effect of a lysate and/or protein hydrolysate, prepared as described herein, from CO₂ as the sole source of carbon entering into the production process, on a cell culture that is provided the lysate and/or protein hydrolysate is performed. The effect on the viability of a LA strain of 2% (w/v) addition of an acid hydrolysate of proteins, produced as described herein, to a milk substrate, is determined. The viability of the LA strain after 12 weeks in terms of colony forming units (CFU)/gram is determined for both the experiments and the controls. The negative control can be the milk substrate alone. The positive controls can include 2% addition of acid casein hydrolysate (ACH) and cysteine. Other controls, positive and negative, can include additions of cysteine alone, ACH alone, whey powder (WP), whey protein concentrate (WPC), tryptone (tryptic digest of casein), soy protein hydrolysates including but not limited to commercial soy PH such as NZ Soy-BL, Hy-soy, and Amisoy, or whole soy protein. Other strains, including S. thermophilus and Bifidobacteria sp., are tested for viability, as well as other performance metrics. In addition to, or instead of, cell viability, other culture metrics that are measured, tested, and compared include specific growth rate, the fermentation time needed to reach pH 4.50, and the titer of lactic acid (g/L).

### Example 8: Evaluating Growth and Acid Production for a Probiotic Strain Grown Using a Protein Hydrolysate

Growth and acid production by a B. lactis strain is evaluated using a medium supplemented with one or more protein hydrolysates produced as described herein. Positive controls for comparison can include ovine and caprine milk as media supplemented with milk protein hydrolysate (MPH) prepared by using commercial protease (e.g., Aspergillus sp. Protease 2A; Amano Pharmaceutical, Nagoya, Japan) (Gomes and Malcata 1998). Free amino acids from various sources, including free amino acid produced as described herein (e.g., via chemoautotrophic conversion of CO₂) can also be used a nitrogen enrichment supplement in experimental and control cases. Free amino acid concentrates are added to the culture medium at proportions of 25-50 mL/L. The viable counts of B. lactis in experimental and control samples are measured and compared.

### Example 9: Evaluating Starter Culture Production Using a Protein Hydrolysate and/or Protein

P. pentosaceus is grown on a substrate. Positive controls are compared against proteins and/or protein hydrolysates produced as described herein as nitrogen sources for the production of P. pentosaceus, including: casein peptone (hydrolyzed casein); primatone (hydrolyzed meat proteins); and/or yeast paste. Performance metrics are measured, including fermentation time and/or biomass yield. The optimal nitrogen/carbon (N/C) ratio is determined.

The effects of various whole cell biomass, lysate, and whole proteins produced as described herein are compared against various dried and frozen meat stocks (1-5%) as growth promoters for Pediococcus acidilactici.

### Example 10: Testing Growth Performance of Different Cell Lines on a Library of Lysates, Hydrolysates, Peptide Compositions and/or Amino Acid Compositions

A library of multiple lysates, hydrolysates, peptide compositions and/or amino acid compositions produced using different feedstocks and methods as described herein are evaluated by compositional analysis and according to growth performance of various model cell types on media comprising members of the library.

Compositional analysis and characterization of the lysates, hydrolysates, peptide compositions and/or amino acid compositions includes: % α-amino nitrogen (AN); % total nitrogen (TN); ratio (AN/TN); free amino acids (% of total amino acid); peptide size ranges e.g. 100-200 Da (%); 200-500 Da (%); 500-1000 Da (%); >1000 Da (%); average MW; ash (%); moisture (%); pH; sodium (%); potassium (%); calcium (mg/g); magnesium (mg/g); chloride (mg/g); sulfate (mg/g); phosphate (mg/g); amino acid content i.e. alanine (mg/g), arginine (mg/g), aspartic acid (mg/g), cysteine (mg/g), glutamic acid (mg/g), glycine (mg/g), histidine (mg/g), isoleucine (mg/g), leucine (mg/g), lysine (mg/g), methionine (mg/g), phenylalanine (mg/g), proline (mg/g), serine (mg/g), threonine (mg/g), tryptophan (mg/g), tyrosine (mg/g), valine (mg/g), and total amino acids (mg/g).

Proliferation of cell types is tested on medium supplemented with one or more lysates, hydrolysates, peptide compositions and/or amino acid compositions produced as described herein. Three model cell types that can be used in such a growth test are: High Five™ cells, derived from cabbage louper, and Sf9 and Sf21 cells, derived from army worm (Spodoptera frugiperda). Each cell line is cultivated in a serum-free medium supplemented (e.g., at a concentration of 0.6% w/v) with test lysate, hydrolysate, peptide composition and/or amino acid composition prior to quantitative performance evaluation. Growth on the supplemented medium is compared against growth on the standard medium for each respective cell type.

### Example 11: Formulation of Cell Culture Media Devoid of Animal Sourced Proteins

Lysates, hydrolysates, peptide compositions and/or amino acid compositions produced as described herein are examined as supplements to a basal medium. The compositions are titrated into basal media. Cell growth with each supplemented medium is compared to the corresponding un-supplemented medium (negative control) or medium supplemented with FBS (positive control). Growth is evaluated in terms of mean cell count per 25 cm² flask over three subcultures. The cell line grown on the experimental and control media can be Vero (African green monkey kidney) cells. Vero cell growth is examined in a prototype serum-free formulation supplemented with various sources of hydrolysates and growth compared following three adaptive passages relative to E-MEM reference medium supplemented with 5% (v/v) FBS. Each lysate, hydrolysate, peptide composition and amino acid composition is supplemented into the medium at 200 mg/L. The comparison is made in terms of mean cell count against FBS-supplemented reference materials.

Comparative yields of target model viruses obtained from Vero cell cultures maintained and infected in either serum-free protein hydrolysates produced as described herein, or serum-supplemented controls, are also examined. Titers of sindbis virus, poliovirus 1, pseudorabies virus, and reovirus produced by VERO cells grown in serum-free medium supplemented with a protein hydrolysate produced as described herein are compared against virus production by VERO cells grown in EMEM with 2% serum as the control. The comparison is made on the basis of plaque-forming units (PFU).

Growth of MDCK (Madin-Darby Canine Kidney) cells and production of canine adenovirus or of infectious bovine rhinotracheitis (IBR) virus in serum-free medium supplemented with a protein hydrolysate produced as described herein is compared against growth and production on E-MEM plus 5% FBS. Growth of PK-15 (Porcine Kidney) cells and pseudorabies virus production in serum-free medium supplemented with a protein hydrolysate produced as described herein is compared against growth and production in E-MEM plus 5% FBS test media. The comparison is made on the basis of cell count and 50% Tissue Culture Infective Dose (TCID50).

### Example 12: Recombinant Protein Production by CHO Cells

A comparative growth experiment of recombinant CHO cells is performed in serum-free medium supplemented with a protein hydrolysate produced as described herein, compared against growth and production in a serum control. The growth is compared in terms of peak cell density and sustained cell viability. Specific protein production rates and volumetric productivity are compared.

### Example 13: Peptide Supplements Tested on Model Cells

The activity of peptide supplements produced as described herein, are tested on a model mouse hybridoma ME-750 producing an IgG2a antibody. The protein-free culture medium is DMEM/F12/RPMI 1640 (3:1:1) supplemented with 0.4 mM HEPES, and 2.0 g/L sodium bicarbonate (Franek, et al., 1992). The control is further supplemented with Basal Medium Eagle (BME) amino acids, 2.0 mM glutamine, survival-promoting amino acids (Franek and S̆rámková, 1996a), and with an iron-rich growth-promoting mixture containing 0.4 mM ferric citrate (Franek, et al., 1992, supra). The experiments to be compared against the control replace one or more of the following supplements: Basal Medium Eagle (BME) amino acids, 2.0 mM glutamine, survival-promoting amino acids (Franek and S̆rámková, 1996a, supra), and/or the iron-rich growth-promoting mixture containing 0.4 mM ferric citrate (Franek, et al. 1992, supra); with a lysate, protein hydrolysate, peptide composition and/or amino acid composition produced as described herein. Another experiment that is compared against the control, is utilizing the full medium including supplements provided to the control as listed above, and then further supplementing this medium with a lysate, protein hydrolysate, peptide composition and/or amino acid composition produced as described herein. The concentration of lysate, protein hydrolysate, peptide composition and/or amino acid composition at inoculation is 0.1%, 0.2%, or 0.3% (w/v).

Another set of experiments tests fed-batch cultures. In the control, a volume of 0.25 mL of a feeding mixture that includes DMEM fortified with 10× BME amino acids, 10× BME vitamins, and 20 mM glutamine is added daily. The experiments to be compared against the control replace one or more of the following supplements: 10× BME amino acids; 10× BME vitamins; and/or 20 mM glutamine; with a lysate, protein hydrolysate, peptide composition and/or amino acid composition produced as described herein

Both batch and fed batch cultures are grown in 25 cm² T-flasks at 37° C. in a humidified atmosphere with 5% CO₂. The culture volume is 6.0 mL.

Monoclonal antibody concentrations are determined by immunoturbidimetry using a calibration curve. The number of apoptotic cells in the cultures is determined by microscopic counting of cells displaying apoptotic morphology—shrunken cells with ruffled membranes. The proportion of cell-cycle phases is determined by permeabilization and staining with propidium iodide.

The batch and fed-batch experiments are compared against the respective controls on the basis of: Viable cells/mL; Culture viability (%); concentration of monoclonal antibody (mg/L). The comparison is on the value of these parameters six days after inoculation.

### Example 14: Production of Omega-7 Oils from CO₂ in a Two-Step Process Via a Protein Hydrolysate Intermediate

In the first stage of the process, protein and/or polyhydroxybutyrate (PHB) is produced on CO₂ and H₂ using Cupriavidus necator. In the second stage, an omega-7 producing microorganism, Rhodococcus opacus, is grown on hydrolyzed protein and/or PHB produced in the first stage, and oil is extracted from the Rhodococcus opacus biomass.

The study focused on the second stage of the process, using test substrates that are known to be similar to those that can be generated from Cupriavidus necator biomass by hydrolysis to demonstrate the ability of Rhodococcus opacus to produce oil containing omega-7's. Various representative substrates were selected for preliminary screening. Tryptone alone, or combined with hydroxy-butyrate produced the highest titer of Rhodococcus opacus. Tryptone served as a proxy for PH derived from C. necator using trypsin hydrolysis was selected for the production of oil.

Rhodococcus opacus was grown on tryptone in a 300-liter bioreactor as a carbon and nitrogen source and produced 560 grams of dried biomass. The omega-7 fraction of the total lipids, based on analysis of an aliquot of biomass before drying, was 15.7% (including 4% palmitoleic and 9% vaccenic acids). The total lipid content of the biomass was 19%, with 9-10% neutral lipids. The oil from the biomass was extracted using a solvent mixture. This process separated the neutral and polar lipids. Around 100 mL of oil was extracted.

### Example 15: Protein Hydrolysates from Chemoautotrophic Sources Replacing Tryptone in Microbiological Media

A number of protein hydrolysates derived from chemoautotrophic sources (e.g., derived from proteins biosynthesized from C1 carbon sources such CO₂, CO, and/or CH₄) are evaluated. Tryptone in Luria-Bertani (LB) broth is replaced with an equal quantity of these alternate protein hydrolysates (PH). Replacement of the tryptone present in LB-medium with various PH is evaluated by growth rate and growth yield of a recombinant Escherichia coli strain. In addition, plasmid stability, inducibility and activity of a plasmid encoded β-galactosidase in the recombinant strain grown in the presence of various protein hydrolysates is evaluated.

### Bacterial Strain

The strain that is used is Escherichia coli ATCC 39114 carrying plasmid POP(UV-5)-3. This plasmid contains the lacZ gene and expresses β-galactosidase to a very high level (up to 15% of total cell protein).

### Media

For routine growth of the culture, LB agar and LB broth are used. LB medium contains tryptone 10 g, yeast extract 5 g and sodium chloride 10 g/L of distilled water.

The culture is stored in glycerated LB broth at −80° C. Tetracycline is used at a concentration of 20 μg/mL. IPTG (Isopropyl-β-D-thiogalactopyranoside) is used at a concentration of 0.5 mM.

A variety of protein hydrolysates replace tryptone in the LB medium. The concentration of each different protein hydrolysate applied is 10 g/L (w/v).

### Growth Studies

Seed cultures are grown aerobically in 5 mL of LB broth, contained in test tubes at 37° C. A 1% inoculum is used to inoculate 20 mL of medium contained in 500 mL sidearm flasks and grown with 250 rpm agitation at 37° C. Growth is measured by optical density at 600 nm.

### β-Galactosidase Assay

A single colony of the culture is inoculated into 5 mL of each respective medium (PH experiments and LB positive control) containing tetracycline (20 μg/mL), contained in test tubes, and incubated at 250 rpm at 37° C. After 8-12 h, a 1% inoculum is transferred into 5 mL of fresh medium of the same composition and grown aerobically with shaking overnight. After overnight growth, a 1% inoculum is transferred into 20 mL of fresh medium contained in a 125 mL Erlenmeyer flask and growth continued until optical densities of 0.35 and 0.7 units, respectively, are reached. β-galactosidase is induced by the addition of IPTG at a final concentration of 0.5 mM. The culture is induced for 30 min at 37° C. with shaking at 250 rpm. Cells grown as described above are assayed for β-galactosidase activity and the units calculated as described by Miller (1992).

### Plasmid Stability

To determine the plasmid stability in various media, a single colony of the culture is inoculated into 5 mL of LB broth, contained in test tubes, and incubated at 250 rpm and 37° C. After overnight growth, a 1% inoculum is transferred into 20 mL of various media (experimental PH media and LB control) contained in 125 mL Erlenmeyer flasks and grown at 250 rpm and 37° C. The culture is grown up to an OD of 1.4. Samples (100 ML) are withdrawn, diluted and plated on various media in the presence and absence of tetracycline. The plates are incubated at 37° C. for 12 h and the colonies counted.

### Properties of the Protein Hydrolysates

The chemical properties of the hydrolysates, such as protein content (%), total amino acid composition and profile, total nitrogen (TN) (%), amino nitrogen (AN) (%), the ratio of amino nitrogen to total nitrogen AN/TN, pH, Ash (%), and moisture content (%) are measured for the experimental media and LB control. The percentage and absolute amino acid composition of the various media are determined for Ala, Arg, Asp, Cys, Glu, Gly, His, Ile, Leu, Lys, Met, Phe, Pro, Ser, Thr, Trp, Tyr, and Val.

The present disclosure is not limited in its application to the details of construction and the arrangement of components set forth in the description. The embodiments of the present disclosure are capable of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Although the foregoing invention has been described in some detail by way of illustration and examples for purposes of clarity of understanding, it will be apparent to those skilled in the art that certain changes and modifications may be practiced without departing from the spirit and scope of the invention, which is delineated in the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. A method for producing a culture medium for culturing microorganisms or cells, comprising:
culturing a first microorganism, thereby producing biomass;
processing the biomass generated by the first cultured microorganism to produce a protein hydrolysate composition; and
adding the protein hydrolysate composition to a culture medium for a second culture comprising a second microorganism or cells, wherein the protein hydrolysate composition serves as a nutrient source for the growth of the second microorganism or cells.

2. The method of claim 0, wherein the processing comprises treating the biomass with one or more of elevated temperature, elevated pressure, and a protease, and/or wherein the processing comprises raising or lowering the pH of a suspension comprising the biomass.

3.-4. (canceled)

5. The method of claim 1, wherein the protein hydrolysate composition comprises a polyhydroxyalkanoate (PHA) and/or comprises a vitamin, and/or wherein the protein hydrolysate composition is enriched in proteins, peptides, and/or amino acids.

6.-9. (canceled)

10. The method of claim 0, wherein said second culture comprises animal cells.

11. The method of claim 10, wherein the culture medium for animal cells is a serum-free culture medium and/or an animal component-free culture medium.

12. (canceled)

13. The method of claim 10, wherein the protein hydrolysate composition serves as a supplement for culturing recombinant animal cells.

14. The method of claim 10, wherein the protein hydrolysate composition serves as a supplement for culturing meat.

15. The method of claim 0, wherein the first microorganism is a chemoautotrophic microorganism, and wherein culturing of the first microorganism comprises culturing the chemoautotrophic microorganism under autotrophic conditions.

16. The method of claim 15, wherein the autotrophic conditions comprises providing a gaseous substrate to grow the chemoautotrophic microorganism, wherein the gaseous substrate comprises one or more of CO₂, CO, and CH₄ as a carbon source and/or one or more of H₂, CO, and CH₄ as an electron donor, and/or wherein the gaseous substrate comprises pyrolysis gas, producer gas, syngas, natural gas or biogas.

17.-19. (canceled)

20. The method of claim 15, wherein the chemoautotrophic microorganism is a knallgas microorganism.

21. The method of claim 15, wherein the chemoautotrophic microorganism is selected from one or more of: Aquifex sp.; Cupriavidus sp.; Corynebacterium sp.; Gordonia sp.; Nocardia sp.; Rhodopseudomonas sp.; Rhodobacter sp.; Rhodospirillum sp.; Rhodococcus sp.; Rhizobium sp.; Thiocapsa sp.; Pseudomonas sp.; Hydrogenomonas sp.; Hydrogenobacter sp.; Hydrogenophilus sp.; Hydrogenovibrio sp.; Hydrogenothermus sp.; Helicobacter sp.; Xanthobacter sp.; Hydrogenophaga sp.; Bradyrhizobium sp.; Ralstonia sp.; Alcaligenes sp.; Amycolata sp.; Aquaspirillum sp.; Arthrobacter sp.; Azospirillum sp.; Variovorax sp.; Acidovorax sp.; Bacillus sp.; Calderobactenum sp.; Derxia sp.; Flavobacterium sp.; Microcyclus sp.; Mycobacterium sp.; Paracoccus sp.; Persephonella sp.; Renobacter sp.; Seliberia sp., Streptomycetes sp.; Thermocrinis sp.; Wautersia sp.; Anabaena sp.; Arthrospira sp.; Scenedesmus sp.; Chlamydomonas sp.; Ankistrodesmus sp.; and Rhaphidium sp.

22. The method of claim 15, wherein the chemoautotrophic microorganism is genetically modified.

23. A cell culture media supplement, comprising a protein hydrolysate composition derived from a microorganism.

24. The media supplement according to claim 23, wherein the microorganism is a chemoautotrophic microorganism.

25. The media supplement of claim 23, wherein the protein hydrolysate composition comprises a vitamin, a PHA, and/or is enriched in proteins, peptides, and/or amino acids.

26.-28. (canceled)

29. A method for culturing animal cells, comprising:
adding a media supplement according to claim 23 to a serum-free culture medium; and
culturing animal cells in the serum-free culture medium.

30. The method of claim 29, wherein the serum-free culture medium is an animal component-free culture medium.

31. The method of claim 29, wherein the culture medium comprises a plant-based and/or yeast-based protein hydrolysate composition.

32. The method of claim 29, wherein the animal cells comprise recombinant cells, stem cells, myoblasts and/or myocytes, or adipocytes and/or fibroblasts.

33.-35. (canceled)

36. The method of claim 29, wherein the culturing comprises contacting the animal cells with a biodegradable and/or consumable scaffold under conditions sufficient for the cells to grow on the scaffold.

37. The method of claim 36, wherein the scaffold comprises a PHA polymer.

38. (canceled)

39. The method of claim 37, wherein the PHA polymer is derived from a chemoautotrophic microorganism.

40. The method of claim 29, wherein said protein hydrolysate composition serves as a supplement for growth and/or differentiation of the animal cells.

41. The media supplement according to claim 23, where the supplement is formulated for use as an animal cell culture media supplement.

42. A method for producing a culture medium for culturing microorganisms or cells, comprising:
culturing a first microorganism, thereby producing biomass, wherein the first microorganism is a chemoautotrophic microorganism;
processing the biomass generated by the first cultured microorganism to produce a protein hydrolysate composition; and
adding the protein hydrolysate composition to a culture medium for a second culture comprising cultured animal cells,
wherein the protein hydrolysate composition serves as a nutrient source for the growth of the second microorganism or cells.

43. The method according to claim 42, wherein the protein hydrolysate composition comprises all essential amino acids, essential vitamins, and growth stimulants for the second culture.

44.-48. (canceled)

49. The method of claim 42, wherein the protein hydrolysate composition serves as a supplement for culturing meat.

50.-56. (canceled)

57. The cell culture media supplement according to claim 23, comprising a protein hydrolysate that is produced by a method comprising:
culturing a chemoautotrophic microorganism, thereby producing biomass; and
processing the biomass generated by the chemoautotrophic microorganism to produce the protein hydrolysate composition.

58. The cell culture media supplement according to claim 57, wherein the protein hydrolysate composition comprises all essential amino acids, essential vitamins, and growth stimulants for the second culture.

59.-62. (canceled)

63. A method for culturing animal cells, comprising:
adding a media supplement according to claim 57 to a serum-free culture medium; and
culturing animal cells in the serum-free culture medium.
